(19) United States
(12) Patent Application Publication
(10) Pub. No.: US 2026/0283466 A1
(43) Pub. Date: Sep. 24, 2026

(54) CAREGIVER ASSISTANCE SYSTEM

(71) Applicant: Stryker Corporation, Portage, MI (US)

(72) Inventors: Ross Michael Nave, Kalamazoo, MI (US); Thomas Joseph Durlach, Kalamazoo, MI (US); John A. Mack, Seattle, WA (US)

(21) Appl. No.: 19/689,131

(22) Filed: May 27, 2026

Related U.S. Application Data
(63) Continuation of application No. 17/913,718, filed on May 20, 2021.
(60) Provisional application No. 63/031,975, filed on May 29, 2020.

(51) Int. Cl. A61B 5/00; G16H 10/60; G16H 40/60

(52) U.S. Cl. CPC A61B 5/0022; A61B 5/002; A61B 5/0077; A61B 5/6891; A61B 5/743; G16H 10/60; G16H 40/60

(57) ABSTRACT

A caregiver assistance system helps caregivers manage the care of patients and the beds that support the patients. The system monitors tasks associated with the patients, such as caregiver rounding tasks, bed sore assessments, and/or fall risk assessments, and forwards completed tasks to an EMR server. The system includes one or more mobile electronic devices that are used to display data about beds and/or the patients assigned to them, and/or to receive data about the beds and/or the patients assigned to them. The system automatically associates data received from the mobile electronic device with a particular patient and/or a particular bed without requiring the user to manually identify the patient or the bed. The data may relate to images of skin conditions of the patient, service requests for the beds, and/or other data. The system may automatically forward service requests to a separate service server that communicates with service personnel.

### REFERENCE TO RELATED APPLICATIONS

This application claims priority to U.S. patent application Ser. No. 17/913,718 filed Sep. 22, 2022, by inventors Ross Michael Nave et al. and entitled CAREGIVER ASSISTANCE SYSTEM, which is a national stage filing of PCT/US 2021/033408 filed May 20, 2021, and which in turn claims priority to U.S. provisional patent application Ser. No. 63/031,975 filed May 29, 2020, the complete disclosures of all of which are incorporated herein by reference.

### BACKGROUND

The present disclosure relates to patient support apparatuses, such as beds, cots, stretchers, recliners, or the like. More specifically, the present disclosure relates to a system for assisting caregivers with both the management of such patient support apparatuses and the performance of other tasks, such as, but not limited to, their rounding tasks.

Hospitals typically expect nurses and/or other caregivers to perform a variety of different duties when caring for patients. These duties include administering medications and/or therapies, taking vital sign readings, installing and removing IV drips, taking blood samples, ensuring patient compliance with prescribed activities and/or medications, assisting the patient into and out of bed, regularly visiting the patient, configuring the patient's bed to be in a desired state, documenting one or more of these activities, and generally being responsive to the patient's needs. One of these duties including performing what are customarily known as patient rounds. Such rounding duties involve the caregiver personally checking on the wellbeing of the patient at certain specified intervals. Hospital administrators typically specify a minimum frequency at which the caregivers are to perform these rounding duties, such as, for example, at least once every two hours. In some situations, the rounding frequencies may vary based on the medical condition of the patient, the wing or section of the hospital, and/or other factors.

Hospitals also typically expect nurses and/or other caregivers to help reduce the risk of a patient developing, or aggravating, bed sores. If a patient develops bed sores, caregivers may be asked to document the location, the size, and/or the progress of the bed sore. Such documentation tasks add to the duties of the caregivers, which may already be quite extensive.

In addition to the foregoing tasks, hospitals also typically expect the nurses to ensure that the patient's bed is configured properly. The proper configuration may involve any one or more of the following: ensuring the bed is plugged into an electrical outlet; ensuring the bed is properly connected to a nurse call system; ensuring an exit detection system is armed; ensuring a monitoring system on the bed is armed; and/or other ensuring one or more other aspects of the bed are in a desired state.

The tasks and responsibilities of nurses and other caregivers are therefore manifold and substantial, and technology that assists these caregivers in meeting their responsibilities and providing quality healthcare to their patients is desirable.

### SUMMARY

According to various embodiments, a tool for assisting caregivers in carrying out multiple ones of their patient care responsibilities is provided herein. According to some embodiments, a tool is specifically provided for facilitating one of more of the following: carrying out bed sore risk reduction and/or documentation duties, carrying out fall risk reduction duties, carrying out bed configuration duties, carrying out rounding duties, and carrying out other periodic duties for which a reminder may be set. The tool may assist with the caregiver's duties by displaying screens that show various status data regarding the bed, as well as notifications and reminders associated with one or more duties of the caregiver. The tool may also be configured to allow caregivers to easily report service needs for particular beds. Still further, the tool may be configured to allow the caregivers to perform their duties without requiring the caregiver to manually enter any information identifying a specific bed and/or a specific patient. Thus, caregivers can use the tool to document patient care and/or bed status data and the tool is configured to automatically associate the documentation and/or bed status data with a specific patient and/or a specific bed. Still other features and functions of the tool are included, as discussed in greater detail below.

A caregiver assistance system according to a first embodiment of the present disclosure includes a plurality of patient support apparatuses and a caregiver assistance application executed on a server. Each of the patient support apparatuses include a litter frame, a support deck supported on the litter frame and adapted to support a patient thereon, a memory, a transceiver, and a controller. The memory contains a unique identifier that uniquely identifies the patient support apparatus. The controller is adapted to transmit that particular patient support apparatus's unique identifier to an offboard device. The caregiver assistance application is adapted to perform the following: (i) communicate with a mobile electronic device comprising a display, a camera, and a user input; (ii) receive from the mobile electronic device a digital image of a particular patient's skin captured by the camera; (iii) identify a particular patient support apparatus to which the particular patient is assigned utilizing correlation data associating the digital image with the particular patient support apparatus; and (iv) send a message to an electronic medical records server in communication with the server. The message includes the digital image and a patient identifier uniquely identifying the particular patient.

According to other aspects of the present disclosure, the caregiver assistance application may be further be adapted to receive the correlation data from the particular patient support apparatus and/or the mobile electronic device.

When the caregiver assistance application is adapted to receive the correlation data from a patient support apparatus, the patient support apparatus may further be adapted to generate the correlation data based on location data received directly from the mobile electronic device when the mobile electronic device is positioned in a common room with that particular patient support apparatus. The uniquely identifies the mobile electronic device and indicates that the digital image was captured while the mobile electronic device was positioned in the common room. The mobile electronic device may also be adapted to automatically transmit the location data to that particular patient support apparatus using wireless communication, wherein the automatic transmission occurs without requiring a user of the mobile electronic device to manually enter any of the location data into the mobile electronic device.

When the caregiver assistance application is adapted to receive the correlation data from the mobile electronic device, the mobile electronic device may further be adapted to wirelessly receive the unique identifier of the particular patient support apparatus while the mobile electronic device is positioned within a common room with a particular patient support apparatus. In such cases, the mobile electronic device is further adapted to generate the correlation data based on the digital image being captured by the mobile electronic device while the mobile electronic device is positioned in the common room with the particular patient support apparatus. In some embodiments, the particular patient support apparatus is adapted to automatically transmit its unique identifier directly to the mobile electronic device when the mobile electronic device is positioned in the common room with the particular patient support apparatus. The automatic transmission occurs without requiring a user of the mobile electronic device to manually manipulate any controls on the particular patient support apparatus. The automatic transmission, in some embodiments, takes place using ultrasonic waves.

In some embodiments, the mobile electronic device is adapted to not transmit to the caregiver assistance application any personal identification data that identifies the particular patient. The mobile electronic device may further be adapted to automatically delete the digital image after transmitting the message to the caregiver assistance application.

In some embodiments, the mobile electronic device is further adapted to measure a physical dimension of a damaged area of the particular patient's skin, such as, but not limited to, a bed sore on the patient's skin. The mobile electronic device may further be adapted to measure the physical dimension by measuring a distance between the particular patient's skin and the mobile electronic device.

In some embodiments, the patient support apparatuses are further adapted to issue an audible alert in response to a triggering condition, and to automatically reduce a volume of the audible alert when the mobile electronic device is moved into the same room as the patient support apparatuses.

In some embodiments, the patient support apparatuses are further adapted to periodically emit a short range transmission of an identification message that contains the unique identifier of the corresponding patient support apparatus. The short range transmission is adapted to not travel outside of a room in which the particular patient support apparatus is positioned. In such embodiments, the mobile electronic device is adapted to detect the short range transmission when the mobile electronic device is positioned within the same room as the emitting patient support apparatus. The mobile electronic device is adapted to forward the unique identifier of the emitting patient support apparatus to the caregiver assistance application while the mobile electronic device is positioned within that room. The short range transmission may be made using ultrasonic waves.

In some embodiments, the mobile electronic device is further adapted to present a service request option on the display that is adapted to allow a user of the mobile electronic device to make a service request for a particular patient support apparatus to the caregiver assistance application. The caregiver assistance application is adapted to forward the service request to a service server that then forwards the service request to at least one service person. The caregiver assistance application may further be adapted to identify the particular patient support apparatus to which the service request pertains by utilizing service correlation data associating the service request with the particular patient support apparatus. The caregiver assistance application is further adapted to receive the service correlation data from the particular patient support apparatus and/or from the mobile electronic device.

When the caregiver assistance application receives the service correlation data from a patient support apparatus, that patient support apparatus may be adapted to generate the service correlation data based on colocation data received directly from the mobile electronic device when the mobile electronic device is positioned in a common room with the particular patient support apparatus. The colocation data uniquely identifies the mobile electronic device and indicates that the service request was made while the mobile electronic device was positioned in the common room. The mobile electronic device may be adapted to automatically transmit the colocation data using wireless communication without requiring a user of the mobile electronic device to manually enter any of the colocation data into the mobile electronic device.

When the caregiver assistance application receives the service correlation data from the mobile electronic device, the mobile electronic device may be adapted to wirelessly receive the unique identifier of a particular patient support apparatus while the mobile electronic device is positioned within a common room with the particular patient support apparatus. In such embodiments, the mobile electronic device is further adapted to generate the service correlation data based on the service request being made while the mobile electronic device is positioned in the common room with the particular patient support apparatus. The particular patient support apparatus may be adapted to automatically transmit its unique identifier directly to the mobile electronic device when the mobile electronic device is positioned in the common room with the particular patient support apparatus. The automatic transmission occurs without requiring a user of the mobile electronic device to manually manipulate any controls on the particular patient support apparatus.

The caregiver assistance application may further be adapted to instruct the mobile electronic device to display rounding data thereon that indicates at least one of: an amount of time since a caregiver last completed a rounding task associated with the particular patient, or an amount of time until the caregiver is supposed to complete a future rounding task associated with the particular patient.

A caregiver assistance system according to a first embodiment of the present disclosure includes a plurality of patient support apparatuses and a caregiver assistance application executed on a server. Each of the patient support apparatuses include a litter frame, a support deck supported on the litter frame and adapted to support a patient thereon, a memory, a transceiver, and a controller. The memory contains a unique identifier that uniquely identifies the patient support apparatus. The controller is adapted to transmit that particular patient support apparatus's unique identifier to an offboard device. The caregiver assistance application is adapted to perform the following: (i) communicate with a mobile electronic device comprising a display, a camera, and a user input, wherein the mobile electronic device is adapted to present a service request option on the display that allows a user of the mobile electronic device to make a service request for a particular patient support apparatus; (ii) receive from the mobile electronic device the service request; (iii) identify the particular patient support apparatus utilizing service correlation data associating the service request with the particular patient support apparatus; and (iv) send a message to a service server in communication with the server. The message includes data identifying both the service request and the particular patient support apparatus.

According to other aspects of the present disclosure, the mobile electronic device may be adapted to automatically transmit the service request to the caregiver assistance application without requiring a user of the mobile electronic device to manually enter any data uniquely identifying the particular patient support apparatus.

In some embodiments, the caregiver assistance application is further adapted to receive a digital image of at least a portion of the particular patient support apparatus from the mobile electronic device, and to forward the digital image to the service server.

In some embodiments, the caregiver assistance application is further adapted to receive the service correlation data from the particular patient support apparatus and/or mobile electronic device.

The caregiver assistance application may further be adapted to instruct the mobile electronic device to display rounding data thereon. The rounding data indicates at least one of: an amount of time since a caregiver last completed a rounding task associated with the particular patient, or an amount of time until the caregiver is supposed to complete a future rounding task associated with the particular patient.

In some embodiments, the caregiver assistance application is further adapted to perform the following: (i) receive from the mobile electronic device a digital image of a particular patient's skin captured by a camera onboard the mobile electronic device; (ii) identify a patient support apparatus to which the particular patient is assigned utilizing correlation data associating the digital image with the assigned patient support apparatus; and (iii) send a health records message to an electronic medical records server in communication with the server. The health records message includes the digital image and a patient identifier uniquely identifying the particular patient.

In some embodiments, the mobile electronic device is adapted to not transmit to the caregiver assistance application any personal identification data that identifies the particular patient. Additionally or alternatively, the mobile electronic device may be further adapted to automatically delete the digital image after transmitting the message to the caregiver assistance application.

In some embodiments, the mobile electronic device is further adapted to measure a physical dimension of a damaged area of the particular patient's skin.

The patient support apparatuses may further be adapted to issue audible alerts in response to a triggering condition, and to automatically reduce a volume of their audible alert when the mobile electronic device is moved into the same room as the patient support apparatuses.

In some embodiments, the patient support apparatuses are further adapted to periodically emit a short range transmission of an identification message that contains the unique identifier of the corresponding patient support apparatus. The short range transmission is adapted to not travel outside of a room in which the emitting patient support apparatus is positioned.

According to another embodiment of the present disclosure, a caregiver assistance system is provided that includes a plurality of patient support apparatuses and a caregiver assistance application adapted to be executed on a server hosted on a local area network of a healthcare facility. Each of the patient support apparatuses includes a litter frame, a support deck supported on the litter frame and adapted to support a patient thereon, a memory, a network transceiver, a short range transmitter, and a controller. The memory contains a unique identifier uniquely identifying the respective patient support apparatus. The network transceiver is adapted to communicate with a local area network. The short range transmitter is adapted to periodically transmit a short range wireless signal that includes the unique identifier and that is adapted to be received by a mobile electronic device when the mobile electronic device is positioned in the same room as the respective patient support apparatus. The controller is adapted to transmit the unique identifier off the respective patient support apparatus via the network transceiver. The caregiver assistance application is adapted to perform the following: (i) receive from the mobile electronic device data regarding at least one of the particular patient support apparatus or a patient assigned to the particular patient support apparatus; (ii) receive from the mobile electronic device the unique identifier of the particular patient support apparatus; and (iii) use the unique identifier of the particular patient support apparatus received from the mobile electronic device to correlate the data with at least one of the particular patient support apparatus or the patient assigned to the particular patient support apparatus.

In some embodiments, the mobile electronic device is adapted to automatically transmit the data to the caregiver assistance application without requiring a user of the mobile electronic device to manually enter any information uniquely identifying the particular patient support apparatus or the patient assigned to the particular patient support apparatus.

In some embodiments, the data relates to a skin condition of the patient assigned to the particular patient support apparatus, and the caregiver assistance application is further adapted to send a message to an electronic medical records server in communication with the server that includes the data and a patient identifier uniquely identifying the patient assigned to the particular patient support apparatus.

In some embodiments, the caregiver assistance application is further adapted to receive from the mobile electronic device a digital image of the skin of the patient assigned to the particular patient support apparatus, wherein the digital image is captured by the camera. The message may include the digital image.

In some embodiments, the mobile electronic device is adapted to not transmit to the caregiver assistance application any personal identification data that identifies the patient assigned to the particular patient support apparatus. The mobile electronic device may further be adapted to automatically delete the digital image after transmitting the message to the caregiver assistance application.

In some embodiments, the data relates to a service request for the particular patient support apparatus, and the caregiver assistance application is further adapted to send a message to a service server in communication with the server. The message includes information identifying both the service request and the particular patient support apparatus. The caregiver assistance application may further be adapted to receive a digital image of at least a portion of the particular patient support apparatus from the mobile electronic device and to forward the digital image to the service server. The digital image may visually capture information relevant to the service request.

In some embodiments, the short range transmitter is an ultrasonic transmitter.

In some embodiments, the particular patient support apparatus is further adapted to issue an audible alert in response to a triggering condition, and to automatically reduce a volume of the audible alert when the mobile electronic device is moved into a common room with the particular patient support apparatus. The particular patient support apparatus may be adapted to detect the presence of the mobile electronic device in the common room by receiving a wireless signal directly from the mobile electronic device. The wireless signal may contain the unique identifier of the particular patient support apparatus.

In some embodiments, the caregiver assistance application is further adapted to instruct the mobile electronic device to display rounding data thereon that indicates at least one of: an amount of time since a caregiver last completed a rounding task associated with the particular patient, or an amount of time until the caregiver is supposed to complete a future rounding task associated with the particular patient.

In still other embodiments, a mobile electronic device and caregiver assistance application are provided that perform any one or more of the functions described herein.

Before the various embodiments disclosed herein are explained in detail, it is to be understood that the claims are not to be limited to the details of operation or to the details of construction and the arrangement of the components set forth in the following description or illustrated in the drawings. The embodiments described herein are capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the claims to any specific order or number of components. Nor should the use of enumeration be construed as excluding from the scope of the claims any additional steps or components that might be combined with or into the enumerated steps or components.

### BRIEF DESCRIPTION OF THE DRAWINGS

FIG. 1 is a perspective view of a patient support apparatus usable in a caregiver assistance system according to one embodiment of the disclosure;

FIG. 2 is a block diagram of a first embodiment of the caregiver assistance system of the present disclosure showing a detailed set of components of the patient support apparatus of FIG. 1, a server-based caregiver assistance application, and a portion of a local area network in which the patient support apparatus is in communication;

FIG. 3 is a block diagram of a set of components of the server-based caregiver assistance application;

FIG. 4 is a block diagram of the caregiver assistance system shown integrated into a local area network of a healthcare facility;

FIG. 5 is a flow diagram of a general algorithm that may be executed by the caregiver assistance application;

FIG. 6 is a plan view of a portable electronic device usable with the caregiver assistance system wherein the portable electronic device is shown displaying a login screen for the caregiver assistance application;

FIG. 7 is an illustrative room listing screen that is displayable on an electronic device of the caregiver assistance system;

FIG. 8 is an illustrative room overview screen that is displayable on an electronic device of the caregiver assistance system;

FIG. 9 is a flow diagram of a skin care documentation algorithm that may be executed by the caregiver assistance application;

FIG. 10 is an illustrative first skin care data input screen that is displayable on an electronic device of the caregiver assistance system;

FIG. 11 is an illustrative skin care image input screen that is displayable on an electronic device of the caregiver assistance system;

FIG. 12 is an illustrative skin care documentation confirmation screen that is displayable on an electronic device of the caregiver assistance system;

FIG. 13 is an illustrative second skin care data input screen that is displayable on an electronic device of the caregiver assistance system;

FIG. 14 is an illustrative third skin care data input screen that is displayable on an electronic device of the caregiver assistance system;

FIG. 15 is an illustrative saved images review screen that is displayable on an electronic device of the caregiver assistance system;

FIG. 16 is an alternative illustrative skin care image input screen that is displayable on an electronic device of the caregiver assistance system;

FIG. 17 is a flow diagram of a screen selection algorithm that may be executed by the caregiver assistance application;

FIG. 18 is a block diagram of a second embodiment of the caregiver assistance system of the present disclosure showing a detailed set of components of a patient support apparatus usable therein, as well as a portion of a local area network in which the patient support apparatus is in communication;

FIG. 19 is a block diagram of a third embodiment of the caregiver assistance system of the present disclosure showing a detailed set of components of a patient support apparatus usable therein, as well as a portion of a local area network in which the patient support apparatus is in communication;

FIG. 20 is a block diagram of a fourth embodiment of the caregiver assistance system of the present disclosure showing a detailed set of components of a patient support apparatus usable therein, as well as a portion of a local area network in which the patient support apparatus is in communication;

FIG. 21 is a block diagram of a fifth embodiment of the caregiver assistance system of the present disclosure showing a caregiver assistance application adapted to operate in conjunction with a remote equipment management system;

FIG. 22 is an illustrative bed identification screen that is displayable on an electronic device of the caregiver assistance system of FIG. 21;

FIG. 23 is an illustrative bed maintenance screen that is displayable on an electronic device of the caregiver assistance system of FIG. 21;

FIG. 24 is an illustrative first service request screen that is displayable on an electronic device of the caregiver assistance system of FIG. 21;

FIG. 25 is an illustrative second service request screen that is displayable on an electronic device of the caregiver assistance system of FIG. 21 and which may be used to capture and input image data into the system;

FIG. 26 is an illustrative third service request screen that is displayable on an electronic device of the caregiver assistance system of FIG. 21 and which may be used to add text to the image data captured via the screen of FIG. 25;

FIG. 27 is an illustrative fourth service request screen that is displayable on an electronic device of the caregiver assistance system of FIG. 21 and which may be used to push a patient support apparatus error to the system; and

FIG. 28 is an illustrative fifth service request screen that is displayable on an electronic device of the caregiver assistance system of FIG. 21 and which may be used to allow a caregiver to manually enter text information regarding a service problem.

### DETAILED DESCRIPTION OF THE EMBODIMENTS

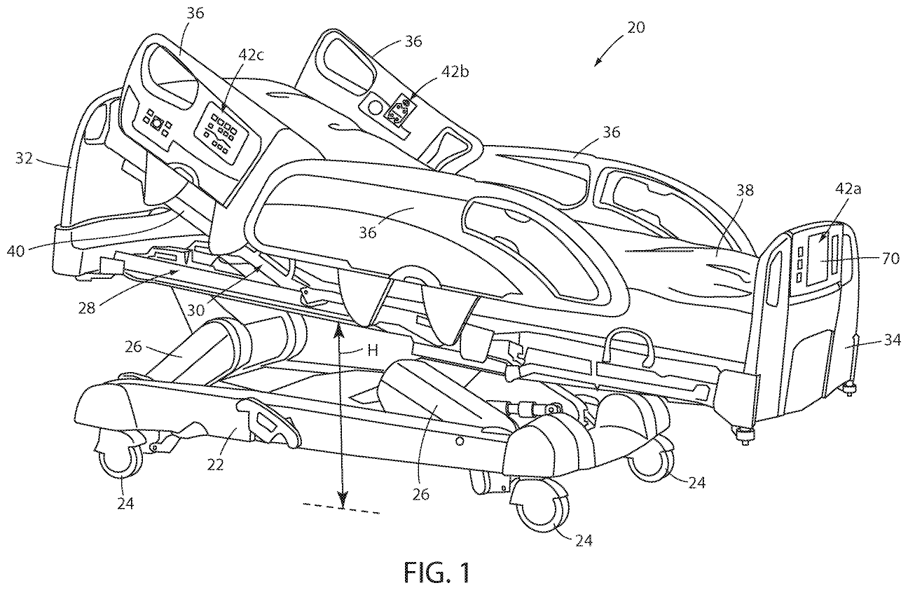

An illustrative patient support apparatus 20 usable in a caregiver assistance system according to the present disclosure is shown in FIG. 1. Although the particular form of patient support apparatus 20 illustrated in FIG. 1 is a bed adapted for use in a hospital or other medical setting, it will be understood that patient support apparatus 20 could, in different embodiments, be a cot, a stretcher, a recliner, or any other structure capable of supporting a patient while the patient is in a healthcare facility, such as, but not limited to, a hospital. For purposes of the following written description, patient support apparatus 20 will be primarily described as a bed with the understanding that the following written description applies to these other types of patient support apparatuses.

In general, patient support apparatus 20 includes a base 22 having a plurality of wheels 24, a lift subsystem comprising a pair of lifts 26 supported on the base, a litter frame 28 supported on the lifts 26, and a support deck 30 supported on the litter frame 28. Patient support apparatus 20 further includes a headboard 32, a footboard 34, and a plurality of siderails 36. Siderails 36 are all shown in a raised position in FIG. 1 but are each individually movable to a lower position in which ingress into, and egress out of, patient support apparatus 20 is not obstructed by the lowered siderails 36. In some embodiments, siderails 36 may be moved to one or more intermediate positions as well.

Lifts 26 are configured to raise and lower litter frame 28 with respect to base 22. Lifts 26 may be hydraulic actuators, electric actuators, or any other suitable device for raising and lowering litter frame 28 with respect to base 22. In the illustrated embodiment, lifts 26 are operable independently so that the tilting of litter frame 28 with respect to base 22 can also be adjusted. That is, litter frame 28 includes a head end and a foot end, each of whose height can be independently adjusted by the nearest lift 26. Patient support apparatus 20 is designed so that when an occupant lies thereon, his or her head will be positioned adjacent the head end and his or her feet will be positioned adjacent the foot end.

Litter frame 28 provides a structure for supporting support deck 30, the headboard 32, footboard 34, and siderails 36. Support deck 30 provides a support surface for a mattress 38, or other soft cushion, so that a person may lie and/or sit thereon. Support deck 30 is made of a plurality of sections, some of which are pivotable about generally horizontal pivot axes. In the embodiment shown in FIG. 1, support deck 30 includes a head section 40, which is also sometimes referred to as a Fowler section or a backrest section. Head section 40 is pivotable about a generally horizontal pivot axis between a generally horizontal orientation (not shown in FIG. 1) and a plurality of raised positions (one of which is shown in FIG. 1). Support deck 30 may include additional sections that are pivotable about one or more horizontal pivot axes, such as an upper leg or thigh section and/or a lower leg or foot section (not labeled).

Patient support apparatus 20 further includes a plurality of control panels 42 that enable a user of patient support apparatus 20, such as a patient and/or an associated caregiver, to control one or more aspects of patient support apparatus 20. In the embodiment shown in FIG. 1, patient support apparatus 20 includes a footboard control panel 42a, a pair of inner siderail control panels 42b (only one of which is visible), and a pair of outer siderail control panels 42c (only one of which is visible). Footboard control panel 42a and outer siderail control panels 42c are intended to be used by caregivers, or other authorized personnel, while inner siderail control panels 42b are intended to be used by the patient associated with patient support apparatus 20. Not all of the control panels 42 include the same controls and/or functionality. In the illustrated embodiment, footboard control panel 42a includes a substantially complete set of controls for controlling patient support apparatus 20 while control panels 42b and 42c include a selected subset of those controls. One or more of any of control panels 42a, b, and/or c may include a height adjustment control that, when activated, changes a height of litter frame 28 relative to base 22.

Control panels 42a and/or 42c may include controls for allowing a user to do one or more of the following: activate and deactivate a brake for wheels 24, arm an exit detection system 46, take a weight reading of the patient, activate and deactivate a propulsion system, and communicate with a healthcare facility computer network installed in the healthcare facility in which patient support apparatus 20 is positioned. Inner siderail control panels 42b may also include a nurse call control that enables a patient to call a nurse. A speaker and microphone are included on, or adjacent to, inner siderail control panel 42b in order to allow the patient to aurally communicate with the remotely positioned nurse.

Footboard control panel 42a is implemented in the embodiment shown in FIG. 1 as a touchscreen display 70 having a plurality of controls 72 positioned alongside the touchscreen display 70. Controls 72 may be implemented as buttons, dials, switches, or other devices. Either or both of control panels 42b or 42c may also include a display for displaying information regarding patient support apparatus 20, and such a display may be a touchscreen in some embodiments. Alternatively, any one or more of control panels 42a-c may omit a touchscreen display and instead include only dedicated controls 72, or some other form of non-display controls.

The mechanical construction of those aspects of patient support apparatus 20 not explicitly described herein may be the same as, or nearly the same as, the mechanical construction of the Model FL27 InTouch Critical Care bed manufactured and sold by Stryker Corporation of Kalamazoo, Michigan. This mechanical construction is described in greater detail in the Stryker Maintenance Manual for the Model FL27 InTouch Critical Care Bed (Version 2.4; 2131-409-002 REV B), published by Stryker Corporation of Kalamazoo, Michigan, the complete disclosure of which is incorporated herein by reference. It will be understood by those skilled in the art that those aspects of patient support apparatus 20 not explicitly described herein can alternatively be designed with other types of mechanical constructions, such as, but not limited to, those described in commonly assigned, U.S. Pat. No. 7,690,059 issued to Lemire et al., and entitled HOSPITAL BED; and/or commonly assigned U.S. Pat. publication No. 2007/0163045 filed by Becker et al. and entitled PATIENT HANDLING DEVICE INCLUDING LOCAL STATUS INDICATION, ONE-TOUCH FOWLER ANGLE ADJUSTMENT, AND POWER-ON ALARM CONFIGURATION, the complete disclosures of both of which are also hereby incorporated herein by reference. The mechanical construction of those aspects of patient support apparatus 20 not explicitly described herein may also take on forms different from what is disclosed in the aforementioned references.

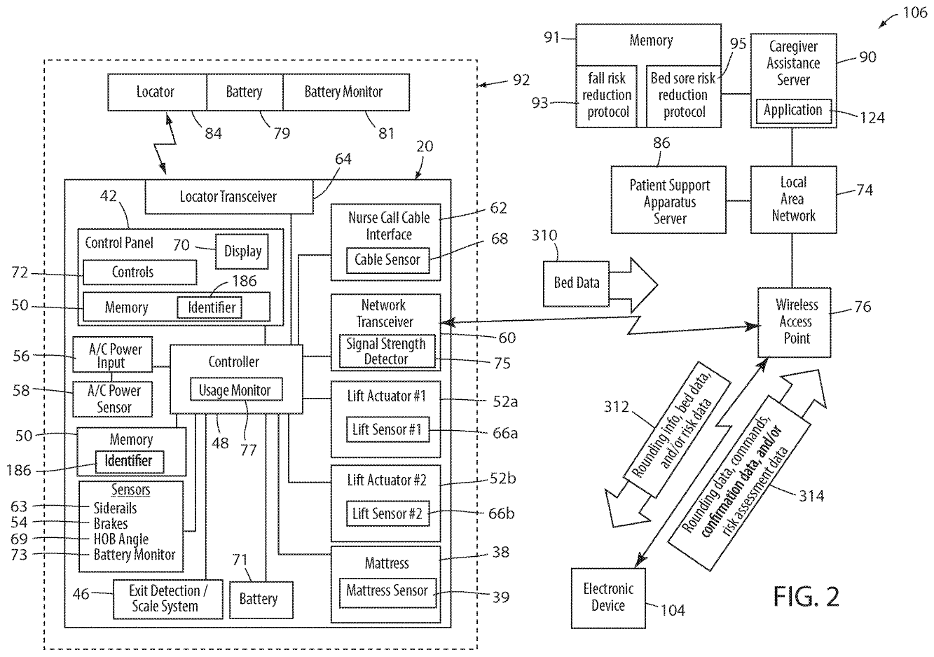

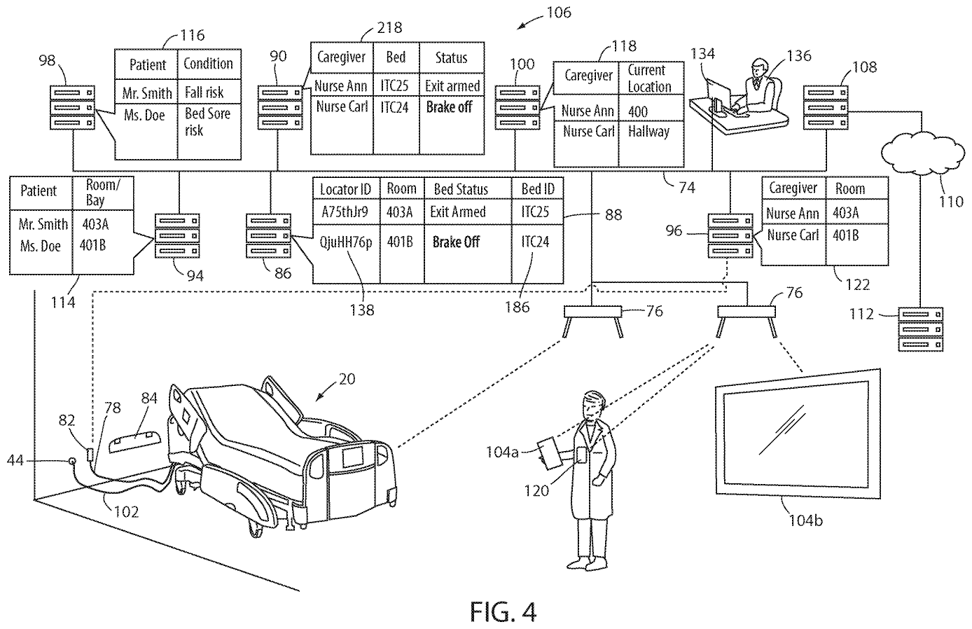

FIG. 2 illustrates a first embodiment of a caregiver assistance system 106 according to the present disclosure. Caregiver assistance system 106 includes patient support apparatus 20 in communication with a caregiver assistance server 90, and one or more electronic devices 104 that are adapted to communicate with caregiver assistance server 90. Caregiver assistance system 106 may also include a conventional patient support apparatus server 86 that is separate from caregiver assistance server 90, or the functionality of caregiver assistance server 90 may be modified to include the functionality of patient support apparatus server 86, thereby allowing patient support apparatus server 86 to be omitted. As will be discussed in greater detail below with respect to FIG. 4, caregiver assistance system 106 communicates with a plurality of conventional servers on a local area network 74 of the healthcare facility and uses those communications to obtain some of the information it needs to perform its caregiver assistance functions.

FIG. 2 illustrates in greater detail some of the internal components of patient support apparatus 20. As shown therein, patient support apparatus 20 includes a controller 48, a memory 50, a first lift actuator 52a, a second lift actuator 52b, a brake sensor 54, an scale/exit detection system 46, an Alternating Current (A/C) power input 56, an A/C power sensor 58, one or more control panels 42, an off-board network transceiver 60 having a signal strength detector 75, a nurse call cable interface 62, a plurality of siderail sensors 63, a location transceiver 64, a head of bed (HOB) angle sensor 69, a battery 71, and a battery monitor 73. Additionally, patient support apparatus 20 includes a first lift sensor 66a, a second lift sensor 66b, a cable sensor 68, display 70, and one or more controls 72 incorporated into one or more of the control panels 42. Still further, patient support apparatus 20 includes a mattress 38 having at least one mattress sensor 39 positioned therein. It will be understood by those skilled in the art that patient support apparatus 20 may be modified to include additional components not shown in FIG. 2, as well modified to include fewer components from what is shown in FIG. 2.

Controller 48 (FIG. 2) is constructed of any electrical component, or group of electrical components, that are capable of carrying out the functions described herein. In many embodiments, controller 48 is a conventional microcontroller, or group of conventional microcontrollers, although not all such embodiments need include a microcontroller. In general, controller 48 includes any one or more microprocessors, field programmable gate arrays, systems on a chip, volatile or nonvolatile memory, discrete circuitry, and/or other hardware, software, or firmware that is capable of carrying out the functions described herein, as would be known to one of ordinary skill in the art. Such components can be physically configured in any suitable manner, such as by mounting them to one or more circuit boards, or arranging them in other manners, whether combined into a single unit or distributed across multiple units as part of an embedded network. When implemented to include an embedded network, the embedded network may include multiple nodes that communicate using one or more of the following: a Controller Area Network (CAN); a Local Interconnect Network (LIN); an I-squared-C serial communications bus; a serial peripheral interface (SPI) communications bus; any of RS-232, RS-422, and/or RS-485 communication interfaces; a LonWorks network, and/or an Ethernet. The instructions followed by controller 48 in carrying out the functions described herein, as well as the data necessary for carrying out these functions, are stored in memory 50, and/or in one or more other memories accessible to the one or more microprocessors, microcontrollers, or other programmable components of controller 48. Memory 50 also includes a unique identifier 186 that uniquely identifies the particular patient support apparatus into which it is incorporated, such as, but not limited to, a serial number.

When controller 48 is implemented to communicate using an on-board Ethernet, the on-board Ethernet may be designed in accordance with any of the Ethernet-carrying patient support apparatuses disclosed in commonly assigned U.S. patent application Ser. No. 14/622,221 filed Feb. 13, 2015, by inventors Krishna Bhimavarapu et al. and entitled COMMUNICATION METHODS FOR PATIENT HANDLING DEVICES, the complete disclosure of which is incorporated herein by reference. In some embodiments, controller 48 may be implemented to include multiple nodes that communicate with each other utilizing different communication protocols. In such embodiments, controller 48 may be implemented in accordance with any of the embodiments disclosed in commonly assigned U.S. patent application Ser. No. 15/903,477 filed Feb. 23, 2018, by inventors Krishna Bhimavarapu et al. and entitled PATIENT CARE DEVICES WITH ON-BOARD NETWORK COMMUNICATION, the complete disclosure of which is incorporated herein by reference.

Controller 48 is shown in FIG. 2 as including a usage monitor 77. Usage monitor 77 monitors the usage of the various components of patient support apparatus 20 and determines if servicing of any of these components should be performed. In some embodiments, usage monitor 77 is carried out purely within controller 48 based upon inputs from the various components of the patient support apparatus 20. In other embodiments, usage monitor 77 may include one or more electrical circuits and/or devices separate from controller 48 that operate in conjunction with controller 48 to monitor the usage, and servicing of, the various components of patient support apparatus 20.

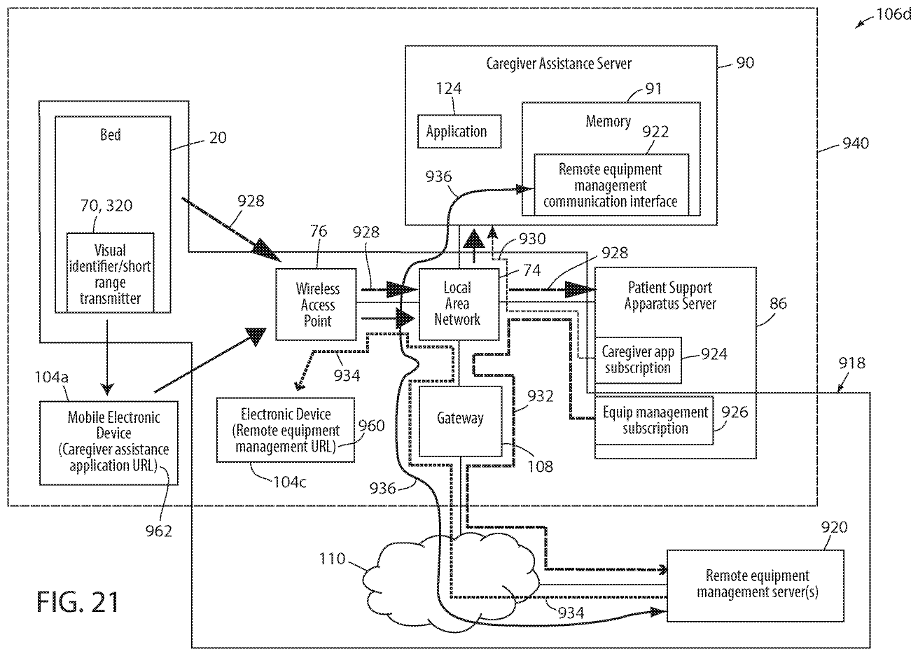

In at least one embodiment, usage monitor 77 includes a data logger that keeps track of the number of times each of the serviceable components (e.g. any component that moves, such as siderails 36, actuators 52, etc.) are moved, activated, or otherwise used. Once the number reaches a threshold—without having been serviced—controller 48 issues a notification and/or alert. The notification/alert may be sent to an electronic device 104 so that caregivers are apprised of the need for servicing one or more components of the patient support apparatus 20. As is also discussed below with respect to FIGS. 21-23, such servicing may be documented using a caregiver assistance application 124 (discussed below), either alone or in conjunction with a standalone equipment management system 918. The documenting of the servicing is forwarded to controller 48, which then resets the appropriate counter(s) and/or other usage parameters.

In some embodiments, usage monitor 77 is implemented to perform the functions of the data logger disclosed in commonly assigned U.S. Pat. No. 7,690,059 issued Apr. 6, 2017, to Lemire et al. and entitled HOSPITAL BED, the complete disclosure of which is incorporated herein by reference. In some embodiments, usage monitor 77 may alternatively, or additionally, perform any of the functions of the diagnostic and control system disclosed in the aforementioned '059 patent. In some embodiments, usage monitor 77 may perform, either alone or in addition to other structures, any of the servicing, monitoring, and/or event detection functions of the equipment management system disclosed in commonly assigned PCT patent publication WO 2018/013666 filed Jul. 12, 2017, by inventors David Becker et al. and entitled EQUIPMENT MANAGEMENT SYSTEM, the complete disclosure of which is incorporated herein by reference. Usage monitor 77 may alternatively or additionally perform still other usage and/or diagnostic monitoring.

First and second lift actuators 52a and 52b (FIG. 2) are components of lifts 26 and are configured to raise and lower litter frame 28 with respect to base 22. A first one of lift actuators 52a powers a first one of the lifts 26 positioned adjacent a head end of patient support apparatus 20 and a second one of lift actuators 52b powers a second one of the lifts 26 positioned adjacent a foot end of patient support apparatus 20. Lift actuators 52a and 52b may be conventional linear actuators having electric motors therein that, when driven, expand or contract the length of the linear actuator, thereby moving the litter frame upward or downward and changing its height H (FIG. 1) relative to the floor.

Each lift actuator 52a and 52b includes a corresponding lift sensor 66a and 66b, respectively. Each of the sensors 66a, 66b detects a position and/or angle of its associated actuator 52a, 52b and feeds the sensed position/angle to controller 48. Controller 48 uses the outputs from sensors 66 as inputs into a closed-loop feedback system for controlling the motion of the actuators 52a, 52b and the litter deck. Controller 48 also uses the outputs from sensors 66a, 66b to determine the height H of litter frame 28 above the floor. In some embodiments, actuators 52 are constructed in any of the same manners as the actuators 34 disclosed in commonly assigned U.S. patent application Ser. No. 15/449,277 filed Mar. 3, 2017, by inventors Anish Paul et al. and entitled PATIENT SUPPORT APPARATUS WITH ACTUATOR FEEDBACK, the complete disclosure of which is incorporated herein by reference. In such embodiments, sensors 66a and 66b may be constructed to include any of the encoders and/or switch sensors disclosed in the aforementioned '277 application.

Scale/exit detection system 46 is configured to determine a weight of a patient positioned on support deck 30 and/or when the patient is moving and is likely to exit patient support apparatus 20. The particular structural details of the exit detection system can vary widely. In some embodiments, scale/exit detection system 46 includes a plurality of load cells arranged to detect the weight exerted on litter frame 28. By summing the outputs from each of the load cells, the total weight of the patient is determined (after subtracting the tare weight). Further, by using the known position of each of the load cells, controller 48 determines a center of gravity of the patient and monitors the center of gravity for movement beyond one or more thresholds. One method of computing the patient's center of gravity from the output of such load cells is described in more detail in commonly assigned U.S. Pat. No. 5,276,432 issued to Travis and entitled PATIENT EXIT DETECTION MECHANISM FOR HOSPITAL BED, the complete disclosure of which is incorporated herein by reference. Other methods for determining a patient's weight and/or the weight of non-patient objects supported on litter frame 28 are disclosed in commonly assigned U.S. patent application Ser. No. 14/776,842, filed Sep. 15, 2015, by inventors Michael Hayes et al. and entitled PATIENT SUPPORT APPARATUS WITH PATIENT INFORMATION SENSORS, and commonly assigned U.S. patent application Ser. No. 14/873,734 filed Oct. 2, 2015, by inventors Marko Kostic et al. and entitled PATIENT SUPPORT APPARATUSES WITH MOTION MONITORING, the complete disclosures of both of which are incorporated herein by reference. Other systems for determining a patient's weight and/or detecting a patient's exit from patient support apparatus 20 may alternatively be used.

Mattress 38 is an inflatable mattress in many embodiments. In some embodiments, mattress 38 includes its own internal controller (not shown) that controls the inflation and deflation of various bladders contained within mattress under the instructions of controller 48. It will therefore be understood that the control of mattress 38 carried out by controller 48 may include both the direct control over the blower(s), pump(s), valve(s), and other components of mattress 38, or an indirect control over on onboard mattress controller that itself carries out the direct controls of the blower(s), pump(s), valve(s), and other components of mattress 38. In either situation, controller 48 may communicate with mattress 38 using a serial cable, or other cable, that extends between patient support apparatus 20 and mattress 38. In at least one alternative embodiment, the communication between patient support apparatus 20 and mattress 38 may be carried out wirelessly, such as in any of the manners disclosed in commonly assigned U.S. Pat. No. 9,289,336 issued to Lambarth et al. and entitled PATIENT SUPPORT WITH ENERGY TRANSFER, the complete disclosure of which is incorporated herein by reference. Other manners for wireless communication may, of course, be used.

In some embodiments, mattress 38 is constructed in accordance with any of the mattresses disclosed in commonly assigned U.S. Pat. No. 9,468,307 issued to Lafleche et al. and entitled INFLATABLE MATTRESS AND CONTROL METHODS, the complete disclosure of which is incorporated herein by reference. In other embodiments, mattress 38 is constructed in accordance with any of the mattresses disclosed in commonly assigned U.S. Pat. No. 8,413,271 issued to Blanchard and entitled PATIENT SUPPORT APPARATUS, the complete disclosure of which is also incorporated herein by reference. Other mattresses may also be used. Regardless of the specific construction of mattress 38, mattress 38 may be configured to carry out one or more different therapy procedures for the patient supported thereon. Such therapy procedures may include, but are not limited to, any of the following: rotation, percussion, vibration, maximum inflation, and turn assistance. Mattress 38 may also be able to be inflated to different states, thereby changing the distribution of pressure on the patient's skin while supported thereon. These various therapies and/or states are often used in order to reduce the likelihood of a patient developing a bed sore, or exacerbating an already existing bed sore. Caregiver assistance system 106 is adapted to suggest, encourage, and/or enforce the utilization of one or more of these therapies and/or states if a patient's skin assessment score is higher than a threshold.

Controller 48 communicates with network transceiver 60 (FIG. 2) which, in at least one embodiment, is a Wi-Fi radio communication module configured to wirelessly communicate with wireless access points 76 of local area network 74. In such embodiments, network transceiver 60 may operate in accordance with any of the various IEEE 802.11 standards (e.g. 802.11b, 802.11n, 802.11g, 802.11ac, 802.11ah, etc.). In other embodiments, network transceiver 60 may include, either additionally or in lieu of the Wi-Fi radio and communication module, a wired port for connecting a network wire to patient support apparatus 20. In some such embodiments, the wired port accepts a category 5e cable (Cat-5e), a category 6 or 6a (Cat-6 or Cat-6a), a category 7 (Cat-7) cable, or some similar network cable, and transceiver 60 is an Ethernet transceiver. In still other embodiments, network transceiver 60 may be constructed to include the functionality of the communication modules 56 disclosed in commonly assigned U.S. patent application Ser. No. 15/831,466 filed Dec. 5, 2017, by inventor Michael Hayes et al. and entitled NETWORK COMMUNICATION FOR PATIENT SUPPORT APPARATUSES, the complete disclosure of which is incorporated herein by reference.

Regardless of the specific structure included with network transceiver 60, controller 48 is able to communicate with the local area network 74 (FIG. 2) of a healthcare facility in which the patient support apparatus is positioned. When network transceiver 60 is a wireless transceiver, it communicates with local area network 74 via one or more wireless access points 76. When network transceiver 60 is a wired transceiver, it communicates directly via a cable coupled between patient support apparatus 20 and a network outlet positioned within the room of the healthcare facility in which patient support apparatus 20 is positioned. As will be discussed in greater detail below with respect to FIG. 4, local area network 74 includes a plurality of servers that are utilized in different manners by the caregiver assistance system disclosed herein, and patient support apparatus 20 communicates with one or more of those servers via transceiver 60 as part of the caregiver assistance system.

When network transceiver 60 is implemented as a wireless transceiver, it may include a signal strength detector 75 that detects the signal strength of the wireless signals it is receiving from the wireless access point 76 with which it is in communication. In some embodiments, such as ones in which network transceiver 60 is a WiFi transceiver, the signal strength detector 75 may be part of the conventional WiFi circuitry that determines the Received Signal Strength Indicator (RSSI), in which case the signal strength may be measured as an RSSI value. In other embodiments, the signal strength may be measured as an actual value in milliwatts (or other units), and signal strength detector 75 may be comprised of any conventional circuitry configured to measure the signal strength in this manner. In still other embodiments, signal strength detector 75 and/or patient support apparatus 20 may have any of the spectrum analysis functionality built into either or both of them that is disclosed in commonly assigned U.S. patent application Ser. No. 15/236,452 filed Sep. 29, 2016, by inventors Krishna Bhimavarapu et al. and entitled PERSON SUPPORT APPARATUSES WITH COMMUNICATION CHANNEL MONITORING, the complete disclosure of which is incorporated herein by reference.

Regardless of whether signal strength detector 75 measures signals using a relative RSSI value or an actual milliwatt value (e.g. −dBm), signal strength detector 75 is configured to forward its results to controller 48 which then displays the value on display 70 and also forwards the value to caregiver assistance application 124 via network transceiver 60. Caregiver assistance application 124 may forward this signal strength value to one or more electronic devices 104 for display thereon.

Nurse call cable interface 62 is an interface adapted to couple to one end of a nurse call cable 78 (FIG. 4). The other end of the nurse call cable 78 couples to a nurse call outlet 82 (FIG. 4) that is typically built into each headwall of each of the patient rooms within a healthcare facility. In many embodiments, nurse call outlet 82 is a 37 pin outlet that cable 78 couples to, thereby enabling patient support apparatus 20 to communicate directly with a conventional nurse call system 80. In some embodiments, nurse call cable interface 62 is constructed in accordance with any of the cable interfaces 92 disclosed in commonly assigned U.S. patent application Ser. No. 15/945,437 filed Apr. 4, 2018, by inventors Krishna Bhimavarapu et al. and entitled PATIENT SUPPORT APPARATUSES WITH RECONFIGURABLE COMMUNICATION, the complete disclosure of which is incorporated herein by reference.

In other embodiments, nurse call cable interface 62 may be replaced with a wireless nurse call communication system that wirelessly communicates with nurse call outlet 82. For example, in some embodiments, nurse call cable interface 62 is replaced with a radio module, such as the radio module 60 disclosed in commonly assigned U.S. patent application Ser. No. 14/819,844 filed Aug. 6, 2015, by inventors Krishna Bhimavarapu et al. and entitled PATIENT SUPPORT APPARATUSES WITH WIRELESS HEADWALL COMMUNICATION, the complete disclosure of which is incorporated herein by reference. In such wireless headwall embodiments, a headwall module, such as headwall module 38 disclosed in the aforementioned '844 application, is included and coupled to nurse call outlet 82. Such a headwall module may replace and/or supplement the functions of location beacon 84, described below. Still other types of wireless communication between the patient support apparatus and nurse call outlet 82 may be implemented.

Regardless of whether nurse call interface 62 uses a wired cable connection to a nurse call outlet on the headwall of the hospital room or it uses a wireless connection, nurse call interface 62 may also, or alternatively, perform any of the functions of the nurse call interfaces disclosed in commonly assigned U.S. patent application Ser. No. 62/833,943 filed Apr. 15, 2019, by inventors Alexander Bodurka et al. and entitled PATIENT SUPPORT APPARATUSES WITH NURSE CALL AUDIO MANAGEMENT, the complete disclosure of which is also incorporated herein by reference.

Siderail sensors 63, which may be conventional siderail sensors, are configured to detect when the siderails 36 are in a raised or lowered position. In most embodiments, a single siderail sensor 63 is included for each of the siderails 36. Therefore, in the embodiment of FIG. 1, patient support apparatus 20 includes four siderail sensors 63, one for detecting the position of each of the four siderails 36. In alternative embodiments, more than one siderail sensor 63 may be included for each siderail 36, such as a first siderail sensor 63 that detects when the siderail is raised and/or locked in its raised position, and a second siderail sensor 63 that detects when the siderail 36 is in its lower position, and/or locked in its lowered position. In general, any switch or other type of sensor that is able to detect when the respective siderail 36 is in its raised and/or locked orientation can be used with patient support apparatus 20. The outputs of siderail sensors 63 are fed to controller 48 and are periodically sent to caregiver assistance server 90 as part of the patient support apparatus status updates that are discussed in greater detail below. Further, as will also be discussed in greater detail below, the position of one or more siderails 36 is monitored for compliance with a desired state, such as, but not limited to, a desired state defined by a fall risk reduction protocol 93 discussed in more detail below.

Location transceiver 64 (FIG. 2) is adapted to detect a wireless signal emitted from a nearby location beacon 84 (FIG. 4) that is positioned at a fixed and known location within the healthcare facility. Although FIG. 4 only illustrates a single one of these location beacons 84, it will be understood that a particular healthcare facility includes many of these location beacons 84 mounted throughout the healthcare facility. Each location beacon 84 includes a wireless short range transmitter (not shown) that broadcasts a wireless, short range signal containing a unique identifier. The short range signal, in some embodiments, is broadcast via an infrared transmitter and is only detectable by receivers (e.g. location transceivers 64) that are positioned within several feet of the location beacon 84. Consequently, location transceivers 64, which are adapted to detect the signals transmitted from location beacons 84, are only able to detect these signals when patient support apparatuses 20 are positioned adjacent (e.g. within several feet) of one of these location beacons 84. If/when location transceiver 64 is able to detect the unique signal from a particular location beacon 84, the corresponding patient support apparatus 20 can therefore be concluded to be currently positioned adjacent that particular location beacon 84. This allows the current location of the patient support apparatus 20 to be identified. In some healthcare facilities, one or more of the patient rooms may not be completely private rooms, but instead may be shared with one or more other patients. In such situations, it is typical to mount two or more location beacons 84 within such a room—one on the headwall at the bay where the first patient support apparatus 20 normally resides and the other on the headwall at the bay where the second patient support apparatus 20 normally resides (and still more if the room is shared by more than two patients).

When location transceiver 64 receives a signal from an adjacent location beacon 84, controller 48 forwards the received signal, including the unique ID of the beacon 84, to a patient support apparatus server 86 (FIG. 2) which is sometimes alternately referred to herein as a bed server 86. Patient support apparatus server 86 includes a location table 88 (FIG. 4), or has access to such a table 88, that correlates beacon IDs to locations within the healthcare facility. Patient support apparatus server 86 is thereby able to determine the location of each patient support apparatus 20 within the healthcare facility (at least all of those that are positioned adjacent a location beacon 84).

In some embodiments, location beacons 84 (FIG. 2) function both as locators and as wireless links to the nurse call outlet 82 integrated into the adjacent headwall. When equipped with this dual function, patient support apparatuses 20 may omit the nurse call cable interface 62, yet still be able to communicate with the nurse call system server 62b. In the illustrated embodiment of FIG. 4, however, patient support apparatus 20 includes a nurse call cable 78 that communicatively couples the patient support apparatus 20 to nurse call outlet 82, thereby enabling the patient support apparatus 20 to communicate directly with the nurse call system 80. Further details about the function of location beacons 84, whether operating solely as locators or both as locators and wireless portals to the nurse call system outlets 82, may be found in any of the following commonly assigned U.S. patent references: U.S. Pat. No. 8,102,254 issued Jan. 24, 2012 to Becker et al. and entitled LOCATION DETECTION SYSTEM FOR A PATIENT HANDLING DEVICE; patent application Ser. No. 14/819,844filed Aug. 6, 2015, by inventors Krishna Bhimavarapu et al. and entitled PATIENT SUPPORT APPARATUSES WITH WIRELESS HEADWALL COMMUNICATION; patent application Ser. No. 62/600,000 filed Dec. 18, 2017, by inventor Alex Bodurka, and entitled SMART HOSPITAL HEADWALL SYSTEM; and patent application Ser. No. 62/598,787 filed Dec. 14, 2017, by inventors Alex Bodurka et al. and entitled HOSPITAL HEADWALL COMMUNICATION SYSTEM, the complete disclosures of all of which are incorporated herein by reference.

Location beacon 84 also includes, in at least some embodiments, a beacon battery 79 and a beacon battery monitor 81. Beacon battery 79 provide electrical power to location beacon 84, either exclusively or, in at least some embodiments, when location beacon 84 is unplugged, or electrical power is otherwise unavailable from an electrical outlet. Beacon battery monitor 81 monitors the charge state of beacon battery 79 and reports measurements of this charge to patient support apparatus 20. That is, the measurements taken by beacon battery monitor 81 are forwarded wirelessly by locator beacon 84 to patient support apparatus 20 via the built-in transmitter of location beacon 84. These measurements are received by location transceiver 64 onboard patient support apparatus 20 and forwarded to controller 48. Controller 48 then displays these measurements on display 70 and/or forwards them to caregiver assistance application 124 via network transceiver 60. Caregiver assistance application 124 may forward these battery charge measurements to one or more electronic devices 104.

In some embodiments, beacon battery monitor 81 may monitor one or more additional factors regarding beacon battery 79, such as, but not limited to, the overall health of beacon battery 79. Such overall health may be measured in terms of the charge capacity of the battery, the number of times the battery has been recharged, the rate at which the battery discharges, the rate at which the battery re-charges, and/or in other manners. In some embodiments, beacon battery monitor 81 may be implemented in the same manner as, and/or configured to monitor and measure any one or more of the same battery parameters as, the battery monitors disclosed in commonly assigned U.S. patent publication 2016/0331614 published Nov. 17, 2016, and filed by inventors Aaron Furman et al. and entitled BATTERY MANAGEMENT FOR PATIENT SUPPORT APPARATUSES, the complete disclosure of which is incorporated herein by reference.

In some embodiments, locator beacon 84 may be incorporated into a wireless headwall module that communicates with patient support apparatus 20 over multiple communication channels. In such embodiments, the first communication channel between location beacon 84 and patient support apparatus 20 may be a short range channel (e.g. infrared) and the second one may be a longer range channel (e.g. Bluetooth). In such embodiments, the transmission of the data from beacon battery monitor 81 to patient support apparatus 20, as well as the transmission of the location identifier of locator beacon 84 to patient support apparatus 20, may occur over either or both of the two communication channels.

Controller 48 of patient support apparatus 20 (FIG. 2) communicates with A/C power sensor 58, which informs controller 48 whether or not an A/C power cable 102 (FIG. 4) is coupled between patient support apparatus 20 and a conventional A/C power outlet 44. In other words, A/C power sensor 58 lets controller 48 know whether patient support apparatus 20 is receiving electrical power from an off-board power supply (e.g. power outlet 44). In some cases, patient support apparatus 20 includes one or more batteries that are able to power patient support apparatus 20, including controller 48, when patient support apparatus 20 is not coupled to a source of electrical power. As will be discussed more below, the status of the A/C power cord 102 (e.g. whether patient support apparatus 20 is operating on battery power or on power from an A/C outlet) is communicated from A/C power sensor 58 to controller 48, which then forwards that status via network transceiver 60 to patient support apparatus server 86 and/or to caregiver assistance server 90.

Controller 48 also communicates with brake sensor 54 (FIG. 2). Brake sensor 54 informs controller 48 whether or not a brake has been applied on patient support apparatus 20. When the brake is applied, one or more of wheels 24 are braked to resist rotation. When the brake is not applied, wheels 24 are free to rotate. As with the data from the A/C power cord sensor 58, the data from the brake sensor 54 is forwarded by controller 48 to patient support apparatus server 86 and/or to caregiver assistance server 90, via network transceiver 60. Caregiver assistance server 90 shares this information with caregivers via one or more electronic devices that are in communication with server 90, as will be discussed in greater detail below.

Controller 48 also communicates with head of bed angle sensor 69. Head of bed angle sensor 69 measure the angular orientation of head section 40 of patient support apparatus 20, either with respect to horizontal or with respect to the general plane of litter frame 28. In some embodiments, head of bed angle sensor 69 is implemented as one or more accelerometers that are mounted to head section 40. In other embodiments, head of bed angle sensor 69 may be implemented as an encoder counter, or other type of counter, that monitors the extension and retraction of the actuator that pivots head section 40. Still other types of sensors may be used to measure the angle of head section 40.

Regardless of the specific type of sensor used for HOB sensor 69, HOB sensor 69 reports its readings to controller 48, which in turn displays them on display 70 and/or forwards them to caregiver assistance application 124 via network transceiver 60. Caregiver assistance application 124 may forward the HOB angle reading to one or more electronic devices 104 for display thereon, thereby enabling caregivers to remotely view the current angle of head section 40 of patient support apparatus 20.

In some embodiments, patient support apparatus 20 may include one or more batteries 71 that are used to provide power to patient support apparatus 20, or certain components thereof, when patient support apparatus 20 is not electrically coupled to a conventional electrical power outlet. For example, in some embodiments, patient support apparatus 20 may include a first battery (or first set of batteries) that are used for all functions on the bed except for powering an onboard propulsion system, and a second battery (or second set of batteries) that are used for powering the onboard propulsion system. One example of such a patient support apparatus 20 is disclosed in commonly assigned U.S. patent application Ser. No. 62/823,324 filed Mar. 25, 2019, by inventors Zane Shami et al. and entitled PATIENT CARE SYSTEM WITH POWER MANAGEMENT, the complete disclosure of which is incorporated herein by reference. In other embodiments, patient support apparatus 20 includes only a single battery 71 (or a single set of batteries 71) that are used for powering all of the electrical functions of patient support apparatus 20. In many instances, whether one or more batteries 71 are included, such batteries 71 are typically rechargeable batteries 71.

In the embodiment shown in FIG. 2, patient support apparatus 20 further includes a battery monitor 73 that is adapted to monitor the charge state (and/or other parameters) of battery 71, or each of the batteries 71 (if there are more than one) of patient support apparatus 20. Battery monitor 73, in addition to monitoring the charge state of one or more batteries 71, may also monitor any of the same parameters of batteries 71 that beacon battery monitor 81 may monitor with respect to beacon battery 79, as discussed above. To that end, battery monitor 73 may be implemented in any of the same manners as, and/or perform any of the same functions as, the battery monitors disclosed in commonly assigned U.S. patent publication 2016/0331614 published Nov. 17, 2016, and filed by inventors Aaron Furman et al. and entitled BATTERY MANAGEMENT FOR PATIENT SUPPORT APPARATUSES, the complete disclosure of which has been incorporated herein by reference.

Regardless of which specific criteria battery monitor 73 monitors for one or more of the batteries 71 of patient support apparatus 20, battery monitor 73 reports the results to controller 48. Controller 48, in turn, displays one or more of the results on display 70 and/or forwards one or more of the results to caregiver assistance application 124. Caregiver assistance application 124 then forwards these results to one or more mobile electronic devices 104a, as discussed in more detail below. The electronic devices 104 include a display and are adapted to display information about the monitored state(s) of battery(ies) 71. If patient support apparatus 20 contains more than one battery, controller 48 forwards the monitored data for each battery (or set of batteries) to caregiver assistance application 124, which in turn forwards this data to one or more electronic devices 104. The recipient electronic device 104 displays the received data separately for each set of batteries. In this manner, caregivers who are remote from patient support apparatus 20 are able to review the status of each of the batteries onboard patient support apparatus 20.

Each of the control panels 42 includes one or more controls 72 that are used to control various functions of the patient support apparatus 20 (FIG. 2). For example, one or more of the control panels 42 includes a motion control 72 for controlling movement of the lift actuators 52a and 52b. Additional controls 72 may be provided for activating and deactivating the brake for wheels 24, arming and disarming exit detection function of scale/exit detection system 46, taking a weight reading of the patient using the scale function of scale/exit detection system 46, activating and deactivating a propulsion system (if included), and communicating with one or more servers on local area network 74. It will be understood that in some embodiments, one or more of controls 72 may be integrated into a touchscreen display, such as display 70. In such embodiments, one or more of the controls may only appear when the user navigates to specific screens displayed on the touchscreen.

Patient support apparatus 20 communicates with the caregiver assistance server 90 of local area network 74 (FIG. 2). Caregiver assistance server 90 is adapted to assist the caregivers in performing a plurality of tasks. In general, caregiver assistance server 90 includes software—a caregiver assistance application 124—that, when executed, assists the caregivers in ensuring that the patient support apparatuses 20 are maintained in a desirable state, assists the caregiver in performing their rounding tasks, assists the caregivers in performing fall and/or skin assessments, assists the caregivers with setting reminders and receiving notifications of the reminders, assists the caregiver with documenting patient conditions and/or patient support apparatus conditions, assists the caregiver in confirming and/or documenting tasks performed by the caregiver, and assists the caregivers with receiving alerts and/or status information about the patients under their care while the caregivers go about their duties.

Caregiver assistance server 90 includes a memory 91 storing various data used by the caregiver assistance application 124 (as well as, in some cases, storing the executable instructions of caregiver assistance application 124). Memory 91 stores such items as, but not limited to, a fall risk reduction protocol 93 and a bed sore risk reduction protocol 95. Memory 91 may be physically included within server 90 and/or it may be distributed across one or more other physical locations that are accessible to server 90.

Caregiver assistance application 124 uses the fall risk reduction protocol 93 when application 124 executes the patient fall risk reduction algorithm 143, as will be discussed in greater detail below. In general, patient fall risk reduction algorithm 143 enables a caregiver to utilize one or more of the electronic devices 104 to assess the fall risk of a patient and to subsequently ensure that the patient support apparatus 20 is in a state specified by the healthcare facility for that patient's particular fall risk. Fall risk reduction protocol 93 specifies the desired state of patient support apparatus 20 for one or more fall risk levels. Fall risk reduction algorithm 143 is discussed in more detail below.

Caregiver assistance application 124 uses the bed sore risk reduction protocol 95 when application 124 executes the patient bed sore risk reduction algorithm 141. In general, patient bed sore risk reduction algorithm 141 enables a caregiver to utilize the same electronic devices used with the fall risk reduction algorithm 143 (and other algorithms discussed herein) to assess the bed sore risk of a patient and to carry out monitoring, compliance, documentation, and implementation of appropriate bed sore risk reductions steps. The particular bed sore risk reduction steps for a particular patient are, in some cases, automatically suggested by caregiver assistance application 124 based upon a bed sore risk score determined for that particular patient. Bed sore risk reduction algorithm 141 is discussed in greater detail below.

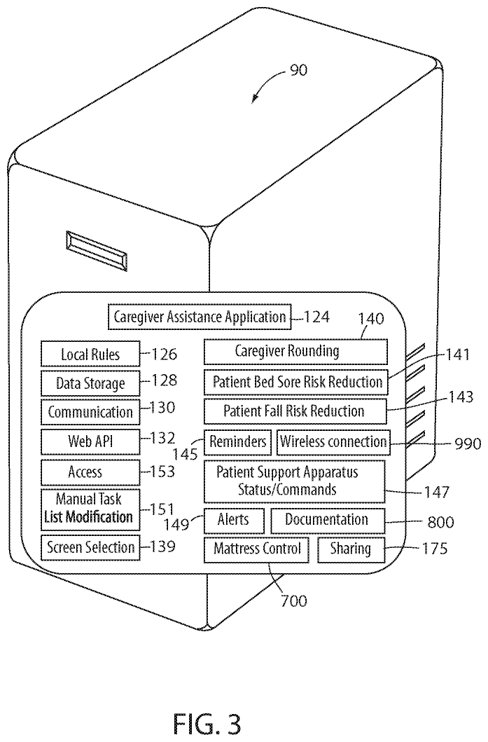

FIG. 3 illustrates in greater detail some of the specific functionality and components of caregiver assistance server 90. Caregiver assistance server 90 is adapted to host and execute a caregiver assistance application 124 that performs a plurality of algorithms and that utilizes a plurality of components. The algorithms includes a screen selection algorithm 139, a caregiver rounding algorithm 140, the bed sore risk reduction algorithm 141, the patient fall risk reduction algorithm 143, a reminder algorithm 145, a status/command algorithm 147, an alerting algorithm 149, a manual task list modification algorithm 151, an access algorithm 153, a sharing algorithm 175, a mattress control algorithm 700, a documentation algorithm 800, and a wireless disconnection detection algorithm 990. Caregiver assistance application 124 may be modified to execute only a subset of these algorithms and/or it may be modified to execute still other algorithms. Further, the content and function of each of these algorithms may be modified from the specific content and functions that are described in more detail below.

Screen selection algorithm 139 automatically selects a type of screen to display on one or more of the mobile electronic devices 104a based upon the location of the caregiver and his/her associated mobile electronic device.

Caregiver rounding algorithm 140 assists a caregiver in performing his or her rounding duties, as well as assisting the caregiver to ensure that patient support apparatuses 20 are properly configured in accordance with the policies of the particular healthcare facility that employs the caregivers and operates the patient support apparatuses 20. In general, caregiver rounding algorithm 140 allows a caregiver to document his or her individual rounding actions while simultaneously reminding the caregiver of any actions that need to be taken to configure the patient support apparatus 20 properly. Such patient support apparatus configurations include, but are not limited to, setting a brake, moving the litter frame to its lowest height (or within a specified range of its lowest height), positioning the siderails in a correct position, arming the exit detection system, plugging in the nurse call cable, plugging in the A/C power cable, and/or arming a patient support apparatus monitoring system.

Bed sore risk reduction algorithm 141 assists the caregiver in assessing a particular patient's risk of developing bed sores and/or in managing the care of a patient's existing bed sores, including documenting both the caregiver's actions and the progress of any bed sores, or other injuries, on the patient. Fall risk reduction algorithm 143 assists the caregiver in assessing the fall risk of a particular patient and/or in ensuring that patient support apparatus 20 is placed in a desired state for reducing the risk of a patient falling. Reminder algorithm 145 assists the caregiver by keeping track of any or all tasks that the caregiver is to complete that have time deadlines, including issuing reminders to the caregiver of when those tasks are due and/or are approaching their deadlines. Status/command algorithm 147 functions to provide the caregivers with up-to-date information of the status of each of the patient support apparatuses 20 having a patient to which that caregiver is assigned, as well as to allow the caregiver to remotely control one or more aspects of those patient support apparatuses 20. Alerting algorithm 149 provides alerts to caregivers when a status of a patient support apparatus 20 is changed to an out-of-compliance state, when a reminder deadline approaches or is reached, and/or whenever any information from any of the other algorithms 140, 141, 143, 145, and/or 147 yields information to which the caregivers should be alerted.

Manual task list modification algorithm 151 allows a caregiver to manually add tasks to, and/or remove tasks from, a task list maintained by caregiver assistance application 124, as well as to change the reminders (timing, frequency, recipients, etc.) that are associated with any of those tasks. Many of the tasks on the task list are automatically populated by one or more of the other algorithms executed by caregiver assistance application 124 (e.g. bed sore risk reduction algorithm 141, fall risk reduction algorithm 143, etc.), but algorithm 151 allows the caregiver to manually modify the list, including tasks on the list that have been automatically populated by caregiver assistance application 124.

Access algorithm 153 controls which individuals are allowed to use caregiver assistance system 106, as well as what information is viewable to the authorized users of caregiver assistance system 106.

Sharing algorithm 175 allows caregivers to share and unshare their responsibilities with each other so that, for example, while a first caregiver is temporarily unavailable, a second caregiver will receive alerts, updates, and other information on his or her mobile electronic device 104a regarding the patient(s) whose first caregiver is temporarily unavailable.

Mattress control algorithm 700 allows a caregiver to remotely control one or more aspects of the mattress 38, including, but not limited to, sending mattress parameters to the mattress 38 that are thereafter stored and utilized by the mattress 38 during a mattress therapy session, or utilized at other times. In some embodiments, mattress control algorithm 700 is also configured to ensure that the mattress settings and/or therapies utilized by a caregiver conform to the healthcare facility's bed sore reduction protocol.

Documentation algorithm 800 allows a caregiver to utilize caregiver assistance application 124 to document various tasks and/or patient data to the patient's corresponding electronic medical record. Such tasks and data include, but are not limited to, tasks and data that relate to the patient's bed sore risks, existing bed sores, and/or other information related to condition and/or treatment of the patient's skin.

Wireless disconnection detection algorithm 990 is adapted to detect when one or more of the patient support apparatuses 20 lose their communication connection to local area network 74 and/or caregiver assistance application 124. In some embodiments, wireless disconnection detection algorithm 990 is adapted to determine not only which patient support apparatuses 20 are not in communication with caregiver assistance application 124, but which ones of those uncommunicative patient support apparatuses 20, if any, are assigned to patients and which ones, if any, are not assigned to patients. By determining this information, wireless disconnection detection algorithm 990 can avoid sending an alert or notification regarding disconnected patient support apparatuses 20 that may be in storage, may be unused, or that otherwise do not have patients assigned to them. This helps reduce alert fatigue for the caregivers by avoiding alerts and/or notifications that do not relate to relevant safety conditions.

Further details regarding the content and operation of screen selection algorithm 139, caregiver rounding algorithm 140, bed sore risk reduction algorithm 141, fall risk reduction algorithm 143, reminder algorithm 145, status/command algorithm 147, alerting algorithm 149, manual task list modification algorithm 151, access algorithm 153, sharing algorithm 175, mattress control algorithm 700, skin documentation algorithm 800, and wireless disconnection detection algorithm 990 are provided in commonly assigned U.S. patent application Ser. No. 62/868,947 filed Jun. 30, 2019, by inventors Thomas Durlach et al. and entitled CAREGIVER ASSISTANCE SYSTEM, the complete disclosure of which is incorporated herein by reference.

In addition to the foregoing algorithms, caregiver assistance application 124 also includes a set of local rules 126, a data repository 128, a communication interface 130, and a web Application Programming Interface 132 (FIG. 3). The set of local rules 126 is initially defined prior to the installation of caregiver assistance application 124 within a particular healthcare facility, in at least some embodiments. In other embodiments, the set of local rules 126 is defined during or after installation of caregiver assistance application 124. In all embodiments discussed herein, however, local rules 126 are modifiable by authorized personnel from the healthcare facility. Such modifications are made by way of one or more computers 134 that are in communication with local area network 74 (FIG. 4). An authorized individual 136 (FIG. 4) utilizes computer 134 to communicate with caregiver assistance application 124 and add, delete, or modify one or more of the local rules 126.

Local rules 126 (FIG. 3) include, but are not limited to, the following: rules indicating how frequently caregivers are to perform their rounding duties (e.g. once every two hours, once every three hours, etc.); rules indicating what state patient support apparatuses 20 are to be placed in (e.g. one or more fall risk reduction protocols 93, bed sore risk reduction protocols 95, VAP protocols, etc.); rules specifying who is to be notified, and when, if a rounding duty is not performed within the desired time period; rules specifying who is to be notified, and when, if a patient support apparatuses is not placed in the desired state and/or is moved out of the desired state; rules specifying how such notifications are to be communicated (e.g. email, phone call, texts, etc.); rules specifying what personnel within the healthcare facility are authorized to view what data using caregiver assistance application 124; and rules specifying if and/or how rounding duties are to be verified and/or documented in the EMR server 98. Both the rules for caregiver assistance frequency and the desired states of the patient support apparatuses 20 may be configured by authorized individuals 136 to vary based upon one or more factors. For example, both the caregiver assistance frequency and desired states of patient support apparatuses may vary for different wings of the healthcare facility, different units of the healthcare facility, different times of day and/or different shifts, different models of patient support apparatuses, different patient health conditions, different patient treatments, different data stored in the EMR server 98, etc.

Local rules 126 (FIG. 3) also include additional administrative data that is stored on caregiver assistance server 90, or stored in a memory otherwise accessible to caregiver assistance application 124. Such administrative data includes, but is not limited to, the IP address, or other network address, of each of the servers with which caregiver assistance application 124 is to communicate (e.g. EMR server 98, ADT server 94, patient support apparatus server 86, RTLS server 100, and nurse call server 96), and/or the IP addresses or other configuration data necessary for caregiver assistance application 124 to communicate with one or more middleware software applications that act as gateways to one or more of these servers. The administrative data also may also include the email addresses, passwords, phone numbers, user names, access levels, and other information about those hospital personnel who have been authorized to use caregiver assistance application 124. The email address and/or phone numbers are used in some embodiments of the alerting algorithm 149 in order for caregiver assistance application 124 to make contact with mobile electronic devices 104a (FIG. 4) carried by the caregivers when there is an alert, or other information to which the caregiver's attention is desirably directed.

Data repository 128 (FIG. 3) stores data that is received by caregiver assistance application 124 during the course of its operation. This data includes patient support apparatus status data sent from patient support apparatuses 20 (via patient support apparatus server 86 in some embodiments, and directly in other embodiments), alert data (e.g. when alerts occurred, causes, remedies, notifications, etc.), rounding completion/incompletion data, verification data verifying caregiver assistance (discussed more below), patient data from bed sore risk reduction and fall risk reduction algorithms 141 and 143, and other data. Data repository 128 may be physically located on server 90 (or another server), or it may be cloud-based, or it may be a combination of both cloud-based storage and local storage maintained at the healthcare facility. Data repository 128 may also store room-to-bed and room bay-to-bed associations that correlate specific patient support apparatuses 20 to specific rooms and/or bays. In some embodiments, it may temporarily also store room-to-patient associations, bed-to-patient associations, caregiver-to-patient associations, caregiver-to-room associations, and/or caregiver-to-patient support apparatus associations.

Communication interface 130 (FIG. 3) controls the communications between caregiver assistance application 124 and the electronic devices 104 with which it is in communication. Communication interface 130 also controls the communications between caregiver assistance application 124 and the servers with which it is in communication. All of these communications, in at least one embodiment, are carried out using conventional Internet packet routing. That is, patient support apparatuses 20 send data in packets that have an IP address corresponding to patient support apparatus server 86 and/or caregiver assistance server 90, and servers 86 and/or 90 send message packets back to patient support apparatuses 20 that include an IP address corresponding to the particular patient support apparatus(es) 20 to which the messages are intended. In some embodiments, each patient support apparatus 20 includes a static IP address that is stored on the patient support apparatus 20, while in other embodiments, the patient support apparatuses 20 consult a local Dynamic Host Configuration Protocol (DHCP) server (not shown) on local area network 74 and the DHCP server assigns a network address to the patient support apparatus.

When communicating with other servers within the healthcare facility, communication interface 130 may utilize different communication protocols, such as, but not limited to, Link Layer Protocol (LLP), Hyper-Text Transfer Protocol Secure (HTTPS), and/or Simple Mail Transfer Protocol (SMTP), etc. In order to facilitate the communication between caregiver assistance server 90 and the other servers of local area network 74, communication interface 130 may utilize a conventional interface engine, such as, but not limited to, the Redox cloud platform that is commercially available from Redox, Inc. of Madison, Wisconsin. Alternatively, or additionally, communication interface 130 may utilize a conventional iGUANA interface engine (HL-7 or otherwise) available from iNTERFACEWARE, Inc. of Toronto, Ontario. Such interfaces allow caregiver assistance application 124 to communicate with different types and/or brands of Electronic Health Record (EHR) systems, such as, but not limited to, those marketed by Cerner corporation, Epic Corporation, Allscripts, etc.

Web API 132 (FIG. 3) provides a portal for authorized devices, software applications and/or servers to access the data of caregiver assistance application 124. In some embodiments, electronic devices 104 communicate with caregiver assistance application 124 via the web API 132 by using a web browser built into the device 104 that accesses one or more Uniform Resource Locators (URLs) that direct the web browser to caregiver assistance application 124. Web API 132, in some embodiments, uses JavaScript Object Notation (JSON) to communicate with the web browsers of the electronic devices 104. In other embodiments, web API 132 use Extensible Markup Language (XML) to communicate with the web browsers of the electronic devices 104. Still other types of communication may be used.

Web API 132 may be configured to communicate with the electronic devices 104 using the conventional GET, POST, DELETE, and UPDATE verbs of the Hyper-Text Transfer Protocol (HTTP). These are used for providing RESTful service (i.e. Representational State Transfers) between web API and the electronic devices 104. For those aspects of caregiver assistance application 124 that utilize two way interactive communication, conventional web socket protocols (e.g. IETF RFC 6455, or the WebSocket API in Web IDL (Interface Description Language) that is standardized by the World Wide Web Consortium (W3C)) may be used for communication between web API 132 and the electronic devices 104. Alternatively, or additionally, conventional pull and push requests may be used for this communication, as well as, but not limited to, server-sent events and/or long polling. Still other communication techniques may be used. In some embodiments, such communications are encrypted such that at least those messages containing patient data are secured against interception. Such encryption takes place, in at least one embodiment, as part of a RESTulf Web service (RWS).

Web API 132 may also be utilized for carrying out additional communications with any of the servers on network 74 and/or for communicating with other software applications that are cloud-based (e.g. equipment management system 918, discussed below) and/or unrelated to caregiver assistance application 124.

In general, caregiver rounding algorithm 140, status/command algorithm 147, and alerting algorithm 149 of caregiver assistance application 124 performs the following functions: gather data from patient support apparatuses 20 about their current states; communicate the patient support apparatus data to electronic devices 104 that are remote from caregiver assistance server 90; cause the electronic devices 104 to display the patient support apparatus status data thereon; cause the electronic devices 104 to display reminders and/or other information on their displays to assist caregivers in performing their rounding tasks, fall prevention tasks, skin care tasks, and other tasks; receive patient data (rounding, skin, fall, etc.) that is input into electronic devices 104 by caregivers during or after the performance of their various tasks; communicate alerts to the caregivers if the patient support apparatus status data indicates the patient support apparatus 20 is not in a desired state or if a timer associated with the patient or the patient support apparatus 20 has expired; forward patient support apparatus commands received from caregivers (via electronic devices 104) to patient support apparatuses 20; receive verification data from electronic devices 104 and/or patient support apparatuses 20 verifying the caregivers'presence adjacent the patient support apparatus 20 when performing the rounding tasks; document to an Electronic Medical Record server 98 (FIG. 4) the successful completion of the caregiver's tasks, as well as the current state of the patient support apparatus status data at the time of completion of the tasks; confirm that the caregiver is following one or more of the healthcare facility's protocols; automatically display a screen on the electronic device that is selected based on the location of the caregiver; temporarily share bed data, alerts, and reminders with other caregivers; and communicate with, and utilize the functionality of, a remote equipment maintenance system that monitors the usage and service history of the beds, as well as other information. Still other functions and features are provided by caregiver assistance application 124. However, it will be understood that, in some embodiments, caregiver assistance application 124 may be modified such that one or more of these functions and/or algorithms are modified, supplemented, and/or omitted. That is, in one embodiment, caregiver assistance application 124 includes only a single one of the algorithms and/or features disclosed herein, in another embodiment it includes all of the algorithms and/or features disclosed herein, and in still other embodiments it includes any combinations of two or more of these algorithms/features but less than all of the algorithms/features.

Patient support apparatus 20 is shown in FIG. 2 positioned in a room 92 of a representative example of a healthcare facility. FIG. 2 also depicts patient support apparatus 20 in communication with local area network 74 of the healthcare facility. It will be understood that the precise structure and contents of the local area network 74 will vary from healthcare facility to healthcare facility. FIG. 4 illustrates in greater detail the contents of a common hospital's local area network 74, along with caregiver assistance server 90 and other components of caregiver assistance system 106.

As shown in FIG. 4, local area network 74 includes a plurality of servers, including a conventional Admission, Discharge, and Tracking (ADT) server 94, a conventional nurse call system server 96, a conventional Electronic Medical Records server 98, a conventional real time location system (RTLS) server 100, and a plurality of conventional wireless access points 76. Local area network 74 also includes caregiver assistance server 90 that, together with one or more patient support apparatuses 20 and one or more electronic devices (e.g. mobile electronic devices 104a or stationary electronic devices 104b) implement one embodiment of the caregiver assistance system 106 according to the present disclosure. Still further, network 74 includes a conventional Internet gateway 108 that couples local area network 74 to the Internet 110, thereby enabling the servers and/or patient support apparatuses 20 to communicate with computers outside of the healthcare facility, such as, but not limited to, a geographically remote server 112. In some embodiments, all or some of the functions of caregiver assistance server 90 are carried out by geographically remote server 112, while in other embodiments caregiver assistance server 90 is configured to implement all of its functions without accessing geographically remote server 112.

ADT server 94 stores patient information, including the identity of patients and the corresponding rooms 92 and/or bays within rooms to which the patients are assigned. That is, ADT server 94 includes a patient-room assignment table 114, or functional equivalent to such a table. The patient-room assignment table correlates rooms, as well as bays within multi-patient rooms, to the names of individual patients within the healthcare facility. The patient's names are entered into the ADT server 94 by one or more healthcare facility staff whenever a patient checks into the healthcare facility and the patient is assigned to a particular room within the healthcare facility. If and/or when a patient is transferred to a different room and/or discharged from the healthcare facility, the staff of the healthcare facility update ADT server 94. ADT server 94 therefore maintains an up-to-date table 114 that correlates patient names with their assigned rooms. ADT server 94 may be a conventional server marketed by Cerner Corporation of North Kansas City, Missouri; EPIC Systems of Madison, Wisconsin; Allscripts Healthcare Solutions, Inc. of Chicago, Illinois; and/or by other companies. Still other types of ADT servers 94 may, of course, be used.

EMR server 98 (FIG. 4) stores individual patient records. Such patient records identify a patient by name and the medical information associated with that patient. Such medical information may include all of the medical information generated from the patient's current stay in the healthcare facility as well as medical information from previous visits. EMR table 116 shows an abbreviated example of two types of medical information entries that are commonly found within a patient's medical records: a fall risk entry indicating whether the patient is a fall risk, and a bed sore risk entry indicating whether the patient is at risk for developing bed sores. Although FIG. 4 shows the data for these entries to be expressed as text, it will be understood that this data may be stored within a medical record in numeric format. For example, the fall risk data may be stored as a numeric value generated from a conventional fall risk assessment tool, such as, but not limited to, the Morse fall risk scale or the Hester-Davis fall risk scale. Similarly, the bed sore data may be stored as a numeric value generated from a conventional bed sore risk assessment tool, such as, but not limited to, the Braden scale. As noted, EMR server 98 includes far more additional information in the medical records of each patient than what is shown in table 116 of FIG. 4, and some of that additional data, such as rounding data, is discussed in more detail below. It will be understood that the term “EMR server,” as used herein, also includes Electronic Health Records servers, or EHR servers for short, and that the present disclosure does not distinguish between electronic medical records and electronic health records. EMR server 98 may be a conventional server marketed by Cerner Corporation of North Kansas City, Missouri; EPIC Systems of Madison, Wisconsin; Allscripts Healthcare Solutions, Inc. of Chicago, Illinois; and/or by other companies. Still other types of EMR servers 98 may, of course, be used.

RTLS server 100 (FIG. 4) is a conventional server that may be present within a given healthcare facility. When present, RTLS server 100 keeps track of the current location of people and equipment within the healthcare facility. In many instances, the RTLS server keeps track of the current location of one or more tags 120 (FIG. 4) that are worn by personnel and/or that are attached to equipment. Such tags 120 may be RF ID tags, or other types of tags. RTLS table 118 provides an example of the type of location data that RTLS server 100 may contain with respect to caregivers. As shown therein, table 118 shows the current location of two caregivers, one by room number (e.g. room 400) and another by general location (e.g. “allway”). Other types of location data may be included. Further, as noted, some healthcare facilities may not include such an RTLS server 100 and caregiver assistance system 106 is able to fully function without such a server. RTLS server 100 may be a conventional Centrak server marketed by Halma plc of Amersham, United Kingdom, or it may be a conventional RTLS server marketed by Stanley Healthcare of Waltham, Massachusetts. Still other types of RTLS servers 100 may, of course, be used.

Nurse call server 96 is shown in FIG. 4 to include a caregiver assignment table 122 that matches caregivers to specific rooms and/or bays within the healthcare facility. Although table 122 only shows caregivers assigned to a single room, it will be understood that each caregiver is typically assigned to multiple rooms. In some nurse call systems 80, caregivers are assigned to specific patients, rather than to specific rooms. Caregiver assistance system 106 is configured to work with both types of nurse call systems 80. Caregiver assistance system 106 is also adapted to work with healthcare facilities that utilize a separate caregiver assignment server (not shown), rather than nurse call server 96, to assign caregivers to rooms and/or patients. Nurse call server 96 may be a conventional server marketed by Rauland (now owned by Ametek, Inc. of Berwyn, Pennsylvania); by West-Com Nurse Call System, Inc. of Fairfield, California; and/or by other companies.

Regardless of whether caregiver assignment table 122 is stored within nurse call server 96 or some other server on network 74, nurse call system server 96 is configured to communicate with caregivers and patients. That is, whenever a patient on a patient support apparatus 20 presses, or otherwise activates, a nurse call, the nurse call signals pass through nurse call cable 78 to nurse call outlet 82. Nurse call outlet 82 is coupled via wire to nurse call server 96 and/or to another structure of nurse call system 80 that then routes the call to the appropriate nurse. The nurse is thereby able to communicate with the patient from a remote location. In some nurse call systems 80, nurse call server 96 is also able to forward alerts and/or other communications to portable wireless devices carried by caregivers and/or to audio stations positioned within patient rooms 92. Such portable wireless devices are the same as mobile electronic devices 104a discussed herein, in at least one embodiment.

Local area network 74 may include additional structures not shown in FIG. 4, such as, but not limited to, one or more conventional work flow servers and/or charting servers that monitor and/or schedule patient-related tasks for particular caregivers, and/or one or more conventional communication servers that forward communications to particular individuals within the healthcare facility, such as via one or more portable devices (smart phones, pagers, beepers, laptops, etc.). The forwarded communications may include data and/or alerts that originate from patient support apparatuses 20 as well as data and/or alerts that originate from caregiver assistance server 90.

Wireless access points 76 are configured, in at least some embodiments, to operate in accordance with any one or more of the IEEE 802.11 standards (e.g. 802.11g, 802.11n, 802.11ah, etc.). As such, patient support apparatuses 20 and electronic devices 104a, 104b that are equipped with Wi-Fi capabilities, and that have the proper authorization credentials (e.g. password, SSID, etc.), can access local area network 74 and the servers hosted thereon. This allows patient support apparatus 20 to send messages to, and receive messages from, patient support apparatus server 86 and/or caregiver assistance server 90. This also allows electronic devices 104 to send messages to, and receive messages from, patient support apparatus server 86 and/or caregiver assistance server 90. As noted previously, alternatively, or additionally, patient support apparatuses 20 may include a wired port for coupling a wired cable (e.g. a Category 5, Category 5e, etc.) between the patient support apparatus 20 and one or more routers/gateways/switches, etc. of network 74, thereby allowing patient support apparatuses 20 to communicate via wired communications with servers 86 and/or 90.

In still other embodiments, one or more of the patient support apparatuses 20 are equipped with alternative wireless transceivers enabling them to communicate directly with patient support apparatus server 86 and/or caregiver assistance server 90 via an antenna and transceiver that is directly coupled to servers 86 and/or 90 and that is separate from LAN 74, thereby allowing patient support apparatuses 20 to bypass LAN 74 in their communications with servers 86 and/or 90. One example of patient support apparatuses equipped to communicate directly with a server on a healthcare facility's local area network without utilizing the LAN is disclosed in commonly assigned U.S. patent application Ser. No. 15/831,466 filed Dec. 5, 2017, by inventors Michael Hayes and entitled NETWORK COMMUNICATION FOR PATIENT SUPPORT APPARATUSES, the complete disclosure of which is incorporated herein by reference. In some embodiments, patient support apparatuses 20 include communication modules, such as the communication modules 66 disclosed in the aforementioned '466 application, and servers 86 and/or 90 are coupled directly to a receiver, such as the enterprise receiver 90 disclosed in the aforementioned '466 application. In such embodiments, patient support apparatuses 20 are able to both send and receive messages directly to and from servers 86 and/or 90 without utilizing access points 76 or any of the hardware of network 74 (other than servers 86 and/or 90).

Caregiver assistance server 90 constructs a table 218 (FIG. 4) that correlates specific caregivers with the patient support apparatuses 20 assigned to them. As shown in FIG. 4, table 218 correlates individual patient support apparatuses 20 and their current statuses to the specific caregivers who are assigned to those patient support apparatuses 20. Although not shown in FIG. 4, table 218 also may correlate caregivers and their patient support apparatuses 20 to specific rooms within the healthcare facility. In order to construct table 218, caregiver assistance application 124 receives the unique patient support apparatus identifiers 186, along with the current status of the patient support apparatuses 20 from patient support apparatus server 86. Caregiver assistance application 124 determines which caregivers are associated with each of these patient support apparatuses 20 based on the caregiver-to-room assignment data it receives from nurse call server 96 (i.e. the data of table 122) and the room-to-patient support apparatus data it receives from patient support apparatus server 86 (i.e. the data from table 88). Caregiver assistance server 90 is therefore supplied with sufficient data to know the current status of each patient support apparatus 20, the room in which each patient support apparatus 20 is assigned, the caregiver assigned to that room and/or patient support apparatus 20, the patient assigned to each patient support apparatus 20, and the fall risk and/or bed sore risk (if known) of each patient. Still further, in those embodiments where an RTLS server 100 is included, caregiver assistance server 90 is also supplied with sufficient data to know the current location of each caregiver.

In some embodiments, caregiver assistance application 124 is configured to determine patient-to-room, patient-to-bed, patient-to-bed-bay, patient-to-caregiver, caregiver-to-room, caregiver-to-patient-support-apparatus, and/or caregiver-to-bed-bay correlations in any of the manners disclosed in commonly assigned U.S. patent application Ser. No. 62/826,097, filed Mar. 29, 2019 by inventors Thomas Durlach et al. and entitled PATIENT CARE SYSTEM, the complete disclosure of which is incorporated herein by reference. In some embodiments, caregiver assistance application 124 may further be modified to carry out any of the staffing errors, and other error-notification functions, disclosed in the aforementioned '097 application.

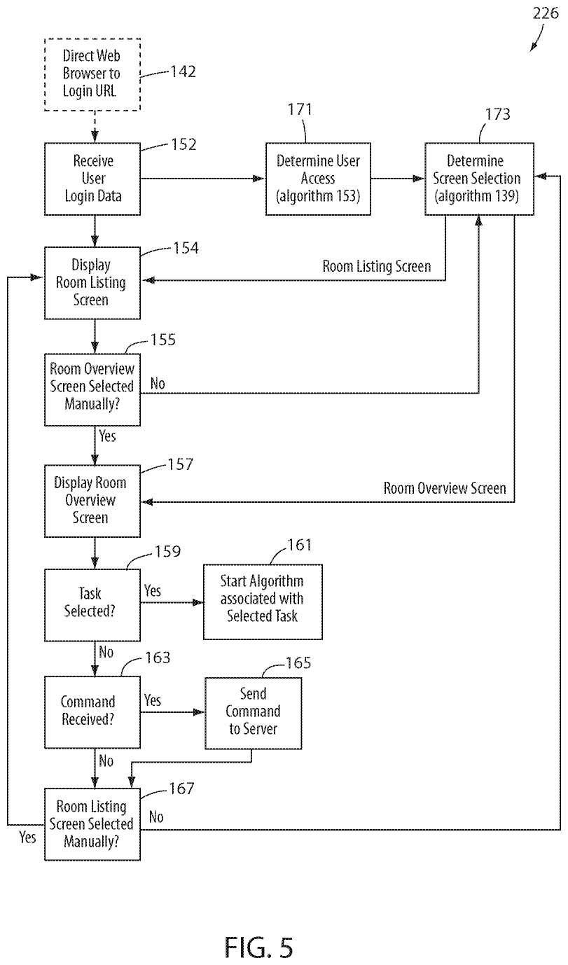

FIG. 5 illustrates a main algorithm 226 executed by caregiver assistance application 124 in at least one embodiment of the present disclosure. Main algorithm 226 is carried out by the one or more processors of caregiver assistance server 90 when caregiver assistance server 90 is executing caregiver assistance application 124. Main algorithm 226 begins at an initial access step 142 where a user accesses caregiver assistance application 124. Initial step 142 is illustrated in FIG. 5 as a window having dashed lines. The dashed lines are presented in order to indicate that step 142 is performed by a user, rather than caregiver assistance application 124 itself. The remaining steps of algorithm 226 are carried out by caregiver assistance application 124.

Initial step 142 is carried out by a user by manipulating one of the electronic devices 104 that are used in conjunction with caregiver assistance application 124. Caregiver assistance system 106 includes one or more electronic devices 104 that communicate with caregiver assistance server 90 and its caregiver assistance application 124. These electronic devices 104 utilize caregiver assistance application 124 to receive status data from patient support apparatuses 20 and to send and receive caregiver assistance data. In other words, caregiver assistance application 124 functions as an intermediary between the electronic devices 104 and the patient support apparatuses 20, as well as an intermediary between the electronic devices 104 and other servers, such as EMR server 98 and/or the nurse call server 96. Caregiver assistance application 124 also performs other functions, as described below.

Electronic devices 104 come in a variety of different forms. As shown in FIG. 4, some electronic devices 104a are mobile electronic devices intended to be carried by a user (e.g. caregiver) while other electronic devices 104b are stationary electronic devices that generally remain in one location. Mobile electronic devices 104a may take on different forms, such as, but not limited to, smart phones, tables, laptop computers, Computers on Wheels (COWs), and others. Stationary electronic devices 104b may also take on different forms, such as, but not limited to, televisions, displays, Personal Computers (PCs), and others. For purposes of the following written description, caregiver assistance system 106 will be described with reference to electronic devices 104 that access caregiver assistance system 106 via a conventional web browser. It will be understood, however, that in other embodiments, electronic devices 104 may be modified to execute a specialized software application that is downloaded to the electronic device 104 and that is tailored to be executed by the particular operating system of the electronic device (e.g. Android, iOS, Windows, etc.). The specialized software application is executed by the microcontroller(s) of the electronic device 104 and carries out the functions of caregiver assistance system 106.

In some embodiments, in order for a caregiver associated with an electronic device 104 to access caregiver assistance system 106, the caregiver utilizes the web-browsing application contained within the electronic device 104 to go to a particular web page, or other URL, associated with caregiver assistance application 124. Any conventional web-browsing software may be used for this purpose, including, but not limited to, Microsoft's Bing or Internet Explorer web browsers, Google's Chrome web browser, Apple's Safari web browser, Mozilla's Firefox web browser, etc. The particular URL accessed with the web browser may vary for different healthcare facilities and can be customized by authorized IT personnel at the healthcare facility. In some embodiments, a domain name may be associated with caregiver assistance application 124 that is resolved by a local DNS server to the IP address of caregiver assistance server 90 (e.g. www. caregiver-assistance-app. com). In other embodiments, electronic devices 104 may include their own native software applications that are programmed to interact with caregiver assistance application 124, thereby avoiding the usage of a web browser to access caregiver assistance application 124. Access to caregiver assistance application 124 may be achieved in other manners. The following description will focus primarily on using a conventional web browser onboard electronic devices 104 to access the caregiver assistance application, but it will be understood that electronic devices 104 may include their own software apps that are specifically tailored to interact with caregiver assistance application 124.

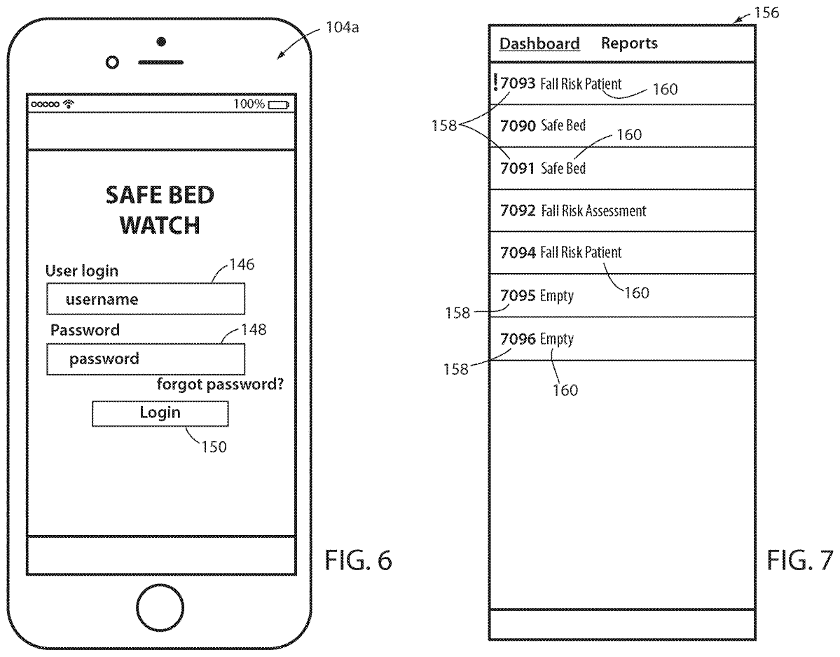

Once at an initial screen, such as a web page corresponding to caregiver assistance application 124, caregiver assistance application 124 instructs the web browser of the electronic device 104 to display a login screen on the display of the electronic device 104. FIG. 6 illustrates an example of such a login screen 144. Login screen 144 is shown in FIG. 6 as being displayed on a mobile electronic device (smart phone) 104a. This is done merely for purposes of illustrating one specific type of electronic device 104 with which caregiver assistance system 106 may be utilized. Other types of devices 104 may be used and the various drawings depict illustrative screens of caregiver assistance system 106 that do not show the specific type of electronic device 104 on which they are displayed, which is intended to re-emphasize the device agnostic nature of electronic device 104 that may be used with caregiver assistance system 106.

Login screen 144 includes a username field 146 in which a user is asked to input his or her username, as well as a password field 148 in which the user is asked to input his or her password. In order for the user to input this information, he or she utilizes the conventional input features of the electronic device 104. Thus, for example, when the electronic device 104 includes a touch screen display and the user touches or otherwise selects either of the fields 146, 148, the electronic device 104 shows on its display, in some embodiments, an image of an alphanumeric keyboard that can be used by the user to input his or her username and password. After this information is typed into fields 146, 148, the user either presses the “enter” or “return” button, or touches the login icon 150 shown on login screen 144. If electronic device 104 does not include a touch screen display, the user may enter the username and login information using a conventional keyboard, a mouse or other pointer, or other methods.

Caregiver assistance application 124 receives the user's username and password at step 152 of main algorithm 226 (FIG. 5). That is, the entry of the user's username and password into electronic device 104 is communicated by the electronic device 104 to caregiver assistance server 90. As was noted, this may be done in a conventional manner utilizing the WiFi, or other network communication, abilities of the electronic device 104. Once caregiver assistance application 124 receives the username and password, it proceeds to step 171 where it determines the level of access, if any, that is to be granted the user. In many embodiments, this determination is carried out in accordance with the steps set forth in access algorithm 153. As discussed in the description of access algorithm 153, caregiver assistance application 124 may utilize rules repository 126 to see if the username and password match an approved user. Local rules repository 126 contains information input into application 124 by an authorized representative of the healthcare facility in which caregiver assistance application 124 is installed. This information includes a list of those individuals who are authorized to use caregiver assistance application 124, including their usernames and passwords (and other data, such as their authorization level, email address, phone number, etc.), as well as what level of access to grant each such individual.

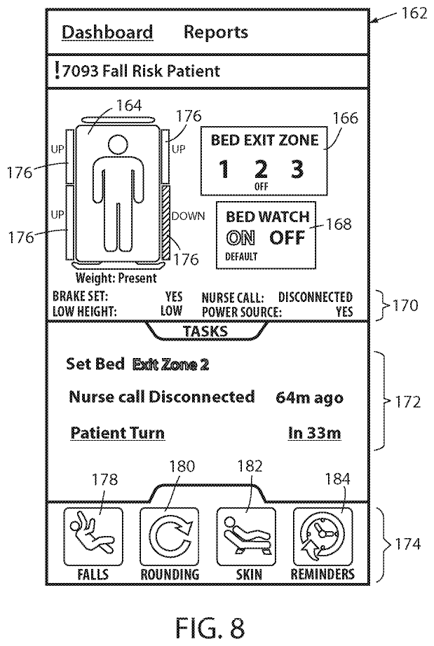

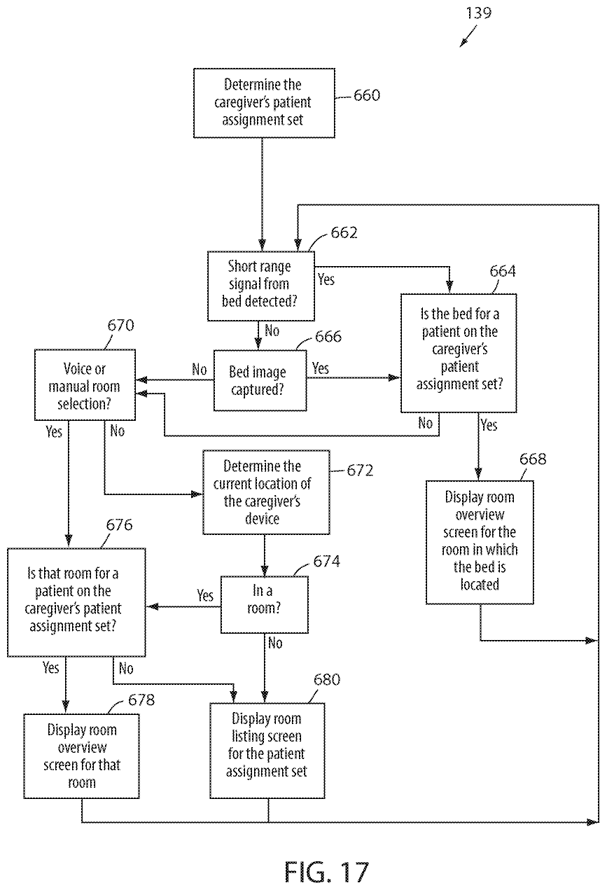

If the user's username and password match an authorized entry within local rules repository 126, caregiver assistance application 124 determines at step 171 (via algorithm 153) what level of access to grant the user. For purposes of the following description of main algorithm 226, it will be assumed that the user is a caregiver, although the user might alternatively be an administrator or a technician (and still other categories are possible). After determining that the user is an authorized caregiver, algorithm 226 proceeds to step 173 where it determines what screen to display on the user's electronic device 104. The selection of a screen to display at step 173 is carried out, in some embodiments, in accordance with screen selection algorithm 139 which, as noted, is described further below with respect to FIG. 17. In the example shown in FIG. 5, algorithm 226 is configured to select between two basic screen types: a room listing screen and a room overview screen. FIG. 7 shows an example of a room listing screen 156, while FIG. 8 shows an example of a room overview screen 162 and 162a. If caregiver assistance application 124 determines at step 173 to display a room listing screen, it proceeds to step 154. If caregiver assistance application 124 determines at step 173 to display a room overview screen, it proceeds to step 157.

As can be seen from FIG. 7, room listing screen 156 displays a plurality of rows and each row includes a room identifier 158 that identifies a particular room 92 within the healthcare facility in which caregiver assistance system 106 is installed. The particular selection of which rooms to list in room listing screen 156 corresponds, in the illustrated embodiment, to the particular person who has just logged into caregiver assistance application 124. That is, each caregiver is assigned a level of administrative access to the data contained within caregiver assistance application 124. This assignment is carried out by one or more of the authorized individuals 136 who initially set up caregiver assistance application 124. In at least one embodiment, caregivers are assigned an access level that only permits them to view rooms that they themselves have been assigned. Caregiver managers may be granted a higher access level that permits them to view all of the rooms of all of the caregivers which they oversee. Administrators may be granted an even higher access that allows them to see all of the rooms in the entire healthcare facility. Still other types of access levels may be used and/or created, and the rules defining the access level architecture are stored within local rules repository 126.

Caregiver assistance application 124 automatically determines which rooms a particular caregiver has been assigned by communicating with a server on local area network 74 that maintains room assignments for caregivers. In the example illustrated in FIG. 4, nurse call server 96 is shown to include a caregiver-room assignment table 122 that stores the room assignments for the caregivers within the healthcare facility. As noted previously, caregiver-room assignment table 122 may be stored on a different server. During installation of caregiver assistance application 124, an authorized administrator inputs the IP address of the server containing caregiver room assignment table 122 (and/or other data necessary to gain access to caregiver-room assignment table 122). Similar data is also input for all of the other servers and tables discussed herein. After a user successfully logins at step 152 of algorithm 140, caregiver assistance application 124 sends a message to the server having caregiver room assignment table 122. The message requests an up-to-date listing of the rooms that are assigned to the caregiver who has just logged in. After receiving this information, caregiver assistance application 124 displays those rooms on the display of the electronic device 104 (or, more precisely, causes the web browser to display those rooms on the display of the electronic device 104). Thus, in the example of FIG. 7, caregiver assistance application 124 displays rooms 7090 through 7096, which correspond to the rooms assigned to the particular caregiver who is using caregiver assistance application 124.

In some healthcare facilities, caregivers may be assigned to specific patients instead of specific rooms. In such instances, caregiver assistance application 124 may be configured in at least two alternative manners. In a first manner, caregiver assistance application 124 continues to display a room listing, such as the room listing screen 156 of FIG. 7. In a second manner, caregiver assistance application 124 displays a patient listing screen that, instead of rows of the rooms the caregiver has been assigned, displays rows of each of the patients the caregiver has been assigned to. When configured in either manner, caregiver assistance application 124 determines the data to display by sending a request to the particular server(s) within the healthcare facility that maintain data sufficient to correlate specific caregivers to specific patients. In the particular embodiment illustrated in FIG. 4, there is no server that correlates patients to caregivers. However, by utilizing patient-room assignment table 114 in conjunction with another server that stores caregiver to room assignments (e.g. table 122), caregiver assistance application 124 is able to determine which particular patients are assigned to a particular caregivers, and which rooms 92 those particular patients are located in within the healthcare facility.

For example, if caregiver assistance application 124 is configured to display room listing screen 156 (FIG. 7) in a healthcare facility that assigns caregivers to specific patients, rather than to specific rooms, caregiver assistance application 124 sends a first request message and a second request message. The first request message is sent to whatever server maintains a table correlating caregivers and the particular patient they have been assigned to care for. The second request is sent to ADT server 94 and requests a listing of the specific rooms in which the caregiver's assigned patients are located. By using the data retrieved from these two requests, caregiver assistance application 124 is able to determine which particular patients the caregivers has been assigned, along with the rooms those patients have been assigned. Caregiver assistance application 124 is thereby able to display room listing screen 156 in a manner that is tailored to the particular caregiver who is using caregiver assistance application 124.

In those embodiments where caregiver assistance application 124 is configured to display rows of the patients assigned to a particular caregiver, rather than the patient room listing screen 156, caregiver assistance application 124 need not send the first request message mentioned above. Instead, it can send a single request message to the particular server that stores the table (or other data structure) that correlates caregivers to particular patients. Caregiver assistance application 124 then displays on the display screen of the electronic device used by that particular caregiver the listing of those patients who are assigned to that particular caregiver.

Still further, in some embodiments, a particular healthcare facility may assign rooms to particular caregivers but may desire to have room listing screen 156 replaced by a patient listing screen that identifies the particular patients assigned to a particular caregiver. Caregiver assistance application 124 may be configured to accommodate this desire. In order to do so, caregiver assistance application 124 sends a message to nurse call server 96 requesting the room assignments for a particular caregiver and also sends a message to ADT server 94 requesting the patient assignments to particular rooms. By using the data from both of these requests, caregiver assistance application 124 is able to determine which patients have been assigned to which caregivers, and is therefore able to display a patient listing screen instead of, or in addition to, room listing screen 156. This is configurable by an authorized individual 136 and is stored in rule repository 126.

It should be noted that, although most electronic devices 104 are associated with a particular caregiver, this is not always the case, particularly for stationary electronic devices 104b. Stationary electronic devices 104b, which may include large screen smart televisions, may be associated with a particular unit of a healthcare facility, a particular nurse's station, wing, floor, and/or other section of the healthcare facility. For these devices, the login credentials may be tailored to the particular location and/or intended function of that particular electronic device 104b. For example, a stationary electronic device 104b may be associated with an oncology unit, an east wing, nurse's station XYZ, the second floor, or rooms A through G, or something else. In such instances, caregiver assistance application 124 may be configured to assign a username and password to each such electronic device 104 that is custom tailored to that specific device. Thus, for example, if a particular electronic device 104 is positioned at a nurse's station within a pediatric oncology unit, the device 104 may be assigned a username of “pediatric oncology display” and have its own specific password. Once an authorized user has logged into caregiver assistance application 124 via that device, caregiver assistance application displays the rooms and/or patient data corresponding to the pediatric oncology unit on that particular device. The room and/or patient data may include rooms and/or patients that are assigned to multiple caregivers, thereby allowing the electronic device 104 to display information beyond that associated with a single caregiver.

Regardless of whether caregiver assistance application 124 displays room listing screen 156 at step 154 or a patient listing screen at step 154 (FIG. 5), caregiver assistance application 124 is also configured to display a status indicator 160 (FIG. 7) on the screen selected at step 154. Status summaries 160 provide additional information about the status of the patient in the room and/or the patient support apparatus 20 assigned to that room. Thus, for example, the status indicator 160 may indicate that a patient is a fall risk or a bed sore risk, that the patient support apparatus 20 is currently empty, that the patient support apparatus 20 is in a compliant or non-compliant state, and/or that one or more tasks (e.g. a fall risk assessment, a skin care assessment, rounding, etc.) are waiting to be performed for that particular patient and/or room.

Caregiver assistance application 124 receives the data necessary for displaying status summaries 160 by communicating with one or more of the servers on local area network 74. In some embodiments, caregiver assistance application 124 receives all of the patient support apparatus data from patient support apparatus server 86, which may be a commercially available bed status server, such as, but not limited to, the iBed server available from Stryker Corporation of Kalamazoo, Michigan. Further details of the iBed server are found in the Stryker Installation/Configuration Manual for the iBed Server 2.0 (document 5212-209-001 REV A), published in May of 2016 by Stryker Corporation of Kalamazoo, Michigan, the complete disclosure of which is incorporated herein by reference. In other embodiments, caregiver assistance application 124 is configured to receive the patient support apparatus status data directly from the patient support apparatuses 20 themselves, rather than through an intermediary server, such as the above-noted iBed server.

Caregiver assistance application 124 receives the patient data and protocol data from EMR server 98 and/or ADT server 94. ADT server 94 may contain, in addition to patient room assignments, requirements data identifying one or more protocols that the healthcare facility requires its caregivers to follow when caring for one or more patients. Such requirements data, for example, may specify what assessments are to be performed on a patient, such as an assessment of the patient's fall risk and/or bed sore risk. Alternatively, such requirements data may be stored elsewhere, such as, but not limited to, local rules repository 126. In some embodiments, the requirements data that specifies which assessments (fall, skin, etc.) are to be performed for a given patient may depend upon the location of the patient within the healthcare facility. For example, some healthcare facilities may configure local rules repository 126 such that all patients within a particular wing, floor, or other section, receive both a fall risk assessment and a skin assessment, while patients within a different location are to receive only one or none of these assessments. Caregiver assistance application 124 automatically checks these local rules when a new patient is admitted to the healthcare facility (as determined from communication with ADT server 94) and, if no assessment is recorded in EMR server 98 (which may be sent there either by caregiver assistance application 124 itself or another device), it displays a reminder on various screens associated with that patient that such an assessment needs to be performed.

Thus, when a new patient enters the healthcare facility, caregiver assistance application 124 automatically determines from server 94 and/or rules repository 126 (or another location) if a particular patient is supposed to have a fall assessment, bed sore assessment, or other assessment performed. If so, caregiver assistance application 124 further sends an inquiry to EMR server 98 to determine if such an assessment has been completed for that particular patient. If it has not, caregiver assistance application 124 displays this lack of completion in the status indicator 160 (FIG. 7). In the example shown in FIG. 7, the patient in room 7092 has not yet had a fall risk assessment performed, and this information is shown in the status indicator 160 corresponding to room 7092.

Turning more particularly to the examples shown in FIG. 7, caregiver assistance application 124 receives the data necessary to indicate that the patient in room 7093 is a fall risk either from EMR server 98 or from data repository 128. Caregiver assistance application 124 requests and receives the data indicating “safe bed” for rooms 7090 and 7091 from patient support apparatus server 86. The term “safe bed” displayed in the status indicator 160 for rooms 7090 and 7091 of FIG. 7 means that the patient support apparatuses 20 in those rooms are currently configured in their desired state. The “desired state” may be a pre-programmed part of caregiver assistance system 106, it may be defined by a fall risk reduction protocol 93, a bed sore risk reduction protocol 95, or another healthcare facility protocol, and/or it may be modified and/or customized by an authorized individual 136. In any of these situations, the definition of the desired state, or “safe bed,” is stored in local rules repository 126. In some embodiments, a particular patient support apparatus 20 is considered to be in the “safe bed” state if all of the following are true: the exit detection system 46 is armed, the brake is activated, the litter frame 28 is at its lowest height (or within a specified range of its lowest height), and at least three of the siderails 36 are in their raised position. As noted, this “safe bed” state may be modified to include, among other things, one or more of the following: a requirement that the A/C cable 102 is plugged into an A/C power outlet; a requirement that the nurse call cable 78 is plugged into the nurse call outlet 82; a requirement that a monitoring function for the patient support apparatus 20 is armed; and/or other requirements. Still further, the “safe bed” state may be modified to remove one or more of the aforementioned criteria.

As was noted previously, caregiver assistance application 124 determines if a patient in a particular room needs to have an assessment performed by checking EMR server 98, data repository 128, and/or one or more other servers on the local area network that define what assessments are to be performed, when they are to be performed, and if they are to be re-performed (and, if so, when). In some embodiments, caregiver assistance application 124 records the completion of one or more of these assessments in data repository 128 in addition to, or in lieu of, sending this data to EMR server 98. As will be discussed in more detail below, one or more of these assessments may be completed, in at least some embodiments, using electronic devices 104 and/or patient support apparatuses 20, and sent to EMR server 98 from either or both of these devices. Alternatively, such assessments may be performed by other devices who forward their results to EMR server 98. In the particular example shown in FIG. 7, caregiver assistance application 124 has determined that the patient in room 7092 has not yet had a fall risk assessment performed, and therefore displays “fall risk assessment” in the status indicator 160 associated with room 7092.

Similarly, caregiver assistance application 124 is configured to display in the status indicator 160 the results of any patient assessments that a caregiver should be aware of. Thus, in the example of FIG. 7, caregiver assistance application 124 displays “fall risk patient” for the status indicator 160 associated with room 7094. This indicates that a fall risk assessment has been performed for the patient in room 7094 and that assessment has indicated that that particular patient is at a higher risk for falling. The results of this fall risk assessment are typically stored in EMR server 98 and/or data repository 128, and caregiver assistance application 124 is configured to request these results from either or both locations and display them in status indicator 160, if a fall risk (or bed sore risk, or other risk) has been detected.

Caregiver assistance application 124 is also configured to display in the status indicator 160 whether or not a patient support apparatus 20 is currently occupied by a patient or not. This information is obtained from the weight sensors, such as load cells, that are included within the scale/exit detection system 46 of each patient support apparatus 20. Each patient support apparatus 20 periodically transmits its weight readings to patient support apparatus server 86. Those weight readings are forwarded to caregiver assistance server 90. If the weight readings are less than a threshold (e.g. 50 pounds), caregiver assistance application 124 concludes that the patient support apparatus 20 is unoccupied and may display this information in status indicator 160 (or it may display other information that is configured to have a higher priority, such as, but not limited to, any assessments that need to be performed for that particular patient). Such information may be displayed in status indicator 160 with the words “weight not detected,” or “patient out of bed,” or some other text that indicates that the patient support apparatus 20 is not detecting the patient.

In the example shown in FIG. 7, caregiver assistance application 124 is displaying the word “empty” for rooms 7095 and 7096. This indicates that those rooms currently do not have any patients assigned to them. Caregiver assistance application 124 determines this information by sending a request to ADT server 94 server asking it for patient information for those rooms 92 that are assigned to the particular caregivers who are using caregiver assistance system 106. In this example, ADT server 94 instructed caregiver assistance application 124 that rooms 7095 and 7096 were not assigned to any patients. Accordingly, caregiver assistance application 124 displays “empty” in the status indicator 160 for these rooms.

It will be understood that the examples of information displayed in the status indicators 160 shown in FIG. 7 are merely several examples of the types of information that may be displayed on room listing screen 156. Caregiver assistance application 124 may be modified to show less, more, and/or different information in status summaries 160 and/or to eliminate them entirely. Still further, caregiver assistance application 124 may be configured to display the status summaries 160 in different colors, depending upon the informational content of the status indicator 160. Thus, for example, tasks that need to be completed may be highlighted in a different color (e.g. orange); information indicating a task has not been complete within a designated time period and/or a patient support apparatus 20 that is out of compliance with a desired state may be highlighted in yet another color (e.g. red); and information indicating that no tasks or no out-of-compliance states exist may be indicated in yet another color (e.g. green). Indications of alerts may be displayed in status summary through flashing text, or still other manners.

Returning to main algorithm 226 of caregiver assistance system 106 (FIG. 5), main algorithm 226 proceeds from step 154 to step 155. At step 155 of main algorithm 226, caregiver assistance application 124 determines whether or not a caregiver has manually selected a particular room from amongst the rooms listed in room listing screen 156. If the caregiver has not manually selected a particular room, algorithm 226 returns to step 173 and re-executes algorithm 139 to determine whether or not to change the screen currently being displayed. The result of the re-execution of algorithm 139 is that caregiver assistance application 124 will either continue to display the room listing screen 156 (by returning to step 154 after re-executing algorithm 139 at step 173), or it will automatically start displaying the room overview screen at step 157. The triggers for this automatic display of the room overview screen are discussed further below with respect to algorithm 139.

Regardless of whether caregiver assistance application 124 arrives at step 157 from step 155 or step 173, caregiver assistance application 124 displays on the screen of electronic device 104 a room overview screen 162, such as the room overview screen 162 of FIG. 8. Thus, if a user manually navigates to (or is automatically brought to) the room listing screen 156 at any point while using caregiver assistance application 124, he or she can press on (or otherwise select) a particular room listed on room listing screen 156. Caregiver assistance application 124 responds to this selection by displaying a room overview screen 162 that corresponds to the particular room 92 selected by the user. The particular room overview screen 162 shown in FIG. 8 is therefore displayed by caregiver assistance application 124 when a user specifically selects room 7093 from room listing screen 156. Caregiver assistance application 124 may also include other tools for allowing a user to navigate to room overview screen 162, such as, but not limited to, a search function in which room numbers may be entered/searched.

Room overview screen 162 (FIG. 8) displays information about a particular room 92 within the healthcare facility and the patient associated with that room 92. It will be understood that room overview screen 162 may be changed to a bay overview screen, or other type of overview screen, if the particular room that the caregiver has selected is a semi-private room containing more than one patient support apparatus 20 or patient. In such embodiments, caregiver assistance application 124 displays a bay overview screen (not shown) similar to room overview screen 162 that is specific to the particular bay that the caregiver has selected the within semi-private room (or it displays an overview screen listing a combination of both rooms and bays).

Room overview screen 162 (or a similar bay overview screen) includes a bed icon 164, an exit detection system status indicator 166, a bed monitoring status indicator 168, a bed status bar 170, a summary area 172, and a task menu 174 (FIG. 8). Bed icon 164 includes a plurality of siderail icons 176 positioned along the sides of bed icon 164. Within each siderail icon 176 is an indicator (not labeled) that includes the word “up” or “down.” Caregiver assistance application 124 selectively displays the “up” or “down” down indication within the siderail icons 176 based upon the current status of the siderails 36 of the patient support apparatus 20 within room 7093. Caregiver assistance application 124 receives the up/down status of each siderail 36 from patient support apparatus server 86 and displays “up” or “down” to match the current siderail status of patient support apparatus 20. Caregiver assistance application 124 is also configured, in at least some embodiments, to display the siderail icons 176 in a different color if they are in the down state, such as, but not limited to, amber. This distinguishes the siderail icons 176 from those corresponding to siderails 36 that are in an up position, which may be displayed in a green color, or some other color.

Exit detection system status indicator 166 (FIG. 8) indicates the current status of the scale/exit detection system 46 of the corresponding patient support apparatus 20 (e.g. the patient support apparatus 20 positioned in room 7093). That is, status indicator 166 indicates if the exit detection system 46 is currently armed or not. It also indicates what zone of the exit detection system the user has selected, if the exit detection system is armed and includes multiple zones. Many exit detection systems are configured to allow a user to select different zones of permitted movement. The different zones allow a patient to move different amounts before the exit detection system issues an alert. In the example of FIG. 8, the patient support apparatus 20 includes an exit detection system 46 having three zones, the second of which is highlighted. The exit detection system 46 is indicated in FIG. 8 as being disarmed (off). Caregiver assistance application 124 displays an “armed” or “on” indicator when the exit detection system 46 is armed, and also highlights the selected zone (1, 2, or 3). Further information about the zones and/or operation of an exit detection system that may be incorporated into patient support apparatus 20 and utilized in caregiver assistance system 106 are found in commonly assigned U.S. patent application Ser. No. 14/918,003 filed Oct. 20, 2015, by inventors Marko Kostic et al. and entitled EXIT DETECTION SYSTEM WITH COMPENSATION, the complete disclosure of which is incorporated herein by reference.

Bed monitoring status indicator 168 (FIG. 8) indicates whether the bed monitoring system of the patient support apparatus 20 is turned on or off. The bed monitoring system is a monitoring feature that is included in some embodiments of patient support apparatuses 20, but may be omitted in other embodiments. In general, the bed monitoring system, when activated, causes controller 48 to monitor the status of a plurality of components of the patient support apparatus 20 to issue an alert when any of those components are changed from a desired state to an undesired state. In several embodiments, the particular features that are monitored by the bed monitoring system are defined by the patient fall risk reduction protocol 93 (FIG. 2). The patient fall risk reduction protocol 93 also defines what the desired states are for each of the particular components that are being monitored by the bed monitoring system.

For example, if the bed monitoring system is activated and includes the monitoring of the siderails 36 of the patient support apparatus 20, controller 48 of patient support apparatus 20 will issue an alert if one or more of the siderails are lowered, or otherwise moved to an undesired state. Generally speaking, when the bed monitoring system is incorporated into a particular patient support apparatus 20, the bed monitoring system will monitor and issue an alert—when armed—if any of the following changes on the patient support apparatus 20 are detected: the exit detection system 46 is disarmed, a siderail 36 is lowered, the patient exits the patient support apparatus 20, the brake is deactivated, the height of the bed is raised beyond a specified level, the A/C power cord 102 is unplugged, and/or the nurse call cable 78 is unplugged. The particular components of the patient support apparatus 20 that are monitored by the bed monitoring system and that, when changed, trigger an alert can be selected by an authorized user, such as authorized individual 136. This selection may take place via one of the control panels 42 of the patient support apparatus 20, one of electronic devices 104, and/or via a computer in communication with caregiver assistance server 90. The alert issued by patient support apparatus 20 in response to the bed monitoring system detecting an undesired state may be a local alert (at patient support apparatus 20), a remote alert (e.g. sent to patient support apparatus server 86 and/or to caregiver assistance application 124), or a combination of both a local and a remote alert. The user may select from these different types of alerts via patient support apparatus 20, electronic devices 104, and/or a computer in communication with server 90.

Bed status bar 170 provides additional information about the current status of patient support apparatus 20 (FIG. 8). This includes an indication of whether or not the brake on the patient support apparatus 20 is activated or not; information indicating whether litter frame 28 is at its lowest height or not; information indicating whether the nurse call cable 78 is plugged into nurse call outlet 82 or not; and information indicating whether the A/C power cable 102 is plugged into an A/C outlet or not. All of the information shown in status bar 170 (as well as all of the patient support apparatus 20 data displayed by caregiver assistance application 124) is sent by the patient support apparatuses 20 (via transceiver 60) to patient support apparatus server 86, which then forwards it to caregiver assistance server 90 and caregiver assistance application 124. Although, in some modified embodiments, caregiver assistance application 124 and caregiver assistance server 90 are configured to receive this information directly from patient support apparatuses 20, thereby avoiding the need for a separate patient support apparatus server 86.

The data displayed in bed status bar 170 (FIG. 8) is updated in real time, or near real time. In most embodiments of patient support apparatuses 20, the patient support apparatuses 20 are configured to automatically (and nearly immediately) communicate their status to patient support apparatus server 86 whenever a change occurs in their status. Thus, for example, if the nurse call cable 78 gets unplugged from the nurse call outlet 82, the patient support apparatus 20 sends a message automatically and almost immediately thereafter to patient support apparatus server 86. The patient support apparatus server 86 automatically, and immediately or nearly immediately, forwards this status update to caregiver assistance application 124. Caregiver assistance application 124, in turn, updates the information displayed in bed status bar 170 to indicate that the nurse call cable has been unplugged. A caregiver, who may be remote from a particular room 92 and/or a particular patient support apparatus 20, thereby gets a real time, or near real time, update of the status of patient support apparatus 20 when utilizing caregiver assistance application 124.

Summary area 172 of room overview screen 162 (FIG. 8) lists one or more items of information about the patient, the patient's patient support apparatus 20, the room assigned to that particular patient, and/or any data generated from the reminder algorithm 145. In the example shown in FIG. 8, the summary area 172 includes a reminder to set, or arm, exit detection system 46, and more specifically to select zone 2 when arming it. This data comes from a task list that is populated by any one or more of the algorithms 140, 141, and 143 and/or by a manual task list modification algorithm 151. The manual task list modification algorithm 151 allows a caregiver to select one or more tasks associated with a patient and/or patient support apparatus 20, schedule those tasks, have reminders issued via caregiver assistance application 124, and display data about those reminders in summary area 172.

Summary area 172 also includes an entry re-iterating the fact that the nurse call cable 78 has been disconnected. Still further, summary area 172 includes an entry reminding the caregiver of any upcoming tasks that are scheduled for this particular patient, room, and/or patient support apparatus 20. In the specific example of FIG. 6, the summary area 172 of room overview screen 162 includes a reminder to turn the patient in room 7093 in thirty-three minutes. This task data is input into caregiver assistance application 124 by a caregiver and/or authorized individual 136 using the manual task list modification algorithm 151 and/or automatically by one or more of the various algorithm 140, 141, and/or 143. The automatically populated reminders include, but are not limited to, reminders to perform a fall risk assessment, to perform a bed sore risk assessment, to perform a rounding duty, to carry out one or more therapies, to configure patient support apparatus 20 and/or various of its components in desired states, etc. The reminders themselves include, in some embodiments, an indication of the amount of time until the task is supposed to be completed (e.g. a time until the next patient turn or next rounding task) and/or an amount of time that has elapsed since the time the task was last completed (e.g. the amount of time since the patient was last turned or the amount of time since the rounding duties were last performed).

Task menu 174 of room overview screen 162 (FIG. 8) identifies a plurality of different tasks that may be undertaken by a caregiver utilizing caregiver assistance application 124. In the example shown in FIG. 8, task menu 174 includes four separate task icons: a fall task icon 178, a rounding task icon 180, a skin task icon 182, and a reminders task icon 184. It will be understood that the number and/or functionality of the tasks listed on task menu 174 may be changed. For example, in some embodiments, task menu 174 is modified to include a service request task option, as will be discussed in greater detail below.

If a caregiver selects one of the task icons 174-182 at step 159, caregiver assistance application 124 begins execution of a corresponding algorithm 140, 141, 143, and 151 at step 161 (FIG. 5). More specifically, if a caregiver selects fall task icon 178 at step 159, caregiver assistance application 124 begins execution of fall risk reduction algorithm 143 at step 161. If a caregiver selects rounding task icon 180 at step 159, caregiver assistance application 124 begins execution of rounding algorithm 140 at step 161. If a caregiver selects skin task icon 182 at step 159, caregiver assistance application 124 begins execution of skin care algorithm 141 (also referred to herein as a bed sore risk reduction algorithm) at step 161. Finally, if a caregiver selects reminder task icon 184 at step 157, caregiver assistance application 124 begins executing manual task list modification algorithm 151 at step 161.

The selection of these various icons and their associated algorithms cause caregiver assistance application 124 to bring up different screens corresponding to the selected task. The different screens enable a user to perform one or more tasks with respect to that particular patient. If the user selects the fall task icon 178, caregiver assistance application 124 begins execution of fall risk reduction algorithm 143 and causes the display of electronic device 104 to display one or more screens allowing a caregiver to perform one or more fall risk reduction steps associated with reducing the likelihood of a patient falling. These steps include, but are not limited to, performing a fall risk assessment and configuring the patient support apparatus 20 according to a fall risk reduction protocol (e.g. in a manner that helps to reduce or minimize a patient's fall risk). The particular screen that is displayed by caregiver assistance application 124 in response to a user selecting the fall task icon 178 (or any of the other task icons of task menu 174) may be an initial screen that is part of a larger set of screens that are displayable by caregiver assistance application 124 in order to assist the caregiver with the selected task.

If a caregiver selects skin task 182 (FIG. 8) at step 159 (FIG. 5), caregiver assistance application 124 proceeds to executes skin care algorithm 141, which causes it to display an initial skin care screen (not shown) that assists the caregiver in performing a bed sore risk assessment, documenting one or more existing skin states or conditions, and/or setting one or more reminders or configurations on the patient support apparatus 20 to assist in preventing the development and/or worsening of a patient's bed sores. As with fall task icon 178, the selection of skin task icon 182 causes caregiver assistance application 124 to display an initial screen associated with caring for a patient's skin that is part of a larger set of screens adapted to assist the caregiver in caring for the patient's skin. The additional screens within that larger set are accessible through the initial screen, or through one or more of the other screens that are accessible from the initial screen.

If a caregiver selects reminder task icon 184 step 159 (FIG. 5), caregiver assistance application 124 proceeds to execute task list modification algorithm 151 and display an initial screen showing existing reminders, such as a room overview screen 162 (FIG. 8) or a room listing screen 156 (FIG. 7). Algorithm 151 thereafter allows the caregiver to set, edit, and/or cancel reminders associated with caring for a particular patient or room. Such reminders include, but are not limited to, reminders to turn the patient, reminders to perform one or more therapies on the patient (e.g. a percussion therapy or maximum inflation therapy using mattress 38), reminders to perform caregiver rounds, and other reminders. Whatever the specific reminder, caregiver assistance application 124 is configured to display the reminder in summary area 172 of room overview screen 162, in the status indicator 160 of room listing screen 156, and/or on other screens of caregiver assistance application 124. The display may include not only an indication of the reminder, but also a time remaining until the reminder deadline is met (or, if the reminder deadline has passed, an amount of time that has passed since the reminder deadline expired). Still further, in some embodiments of caregiver assistance system 106, caregiver assistance application 124 is configured to send a notification to the caregiver when a reminder deadline is reached (or at one or more configurable times ahead of the reminder deadline and/or at one or more configurable times after the reminder deadline if the task remains uncompleted). The notifications include, in some embodiments, an email, a text, a phone call, or some other type of notification, as will be discussed more below.

During the performance of any of the tasks identified in task menu 174, caregiver assistance application 124 is configured to continue to display task menu 174 on the screens that are specifically associated with those tasks. If the user selects a task icon corresponding to a task different from the one currently being executed, caregiver assistance application 124 switches to performing the algorithm associated with that particular task.

If the caregiver does not select any of the tasks from task menu 174, main algorithm 226 (FIG. 5) of caregiver assistance application 124 proceeds to step 163 where it determines if a caregiver has input a command to control one or more aspects of the patient support apparatus 20. If the caregiver has input such a command, algorithm 226 proceeds to step 165 where it sends the command to the patient support apparatus 20. The routing of this command is through caregiver assistance server 90, in at least one embodiment. That is, the command to control one or more aspects of the patient support apparatus 20 is sent from the electronic device 104 to caregiver assistance application 124 (via one or wireless access points 76). After being received, caregiver assistance application 124 forwards the command either directly to the corresponding patient support apparatus 20 using wireless access points 76, or it forwards the command to patient support apparatus server 86, which then forwards the command to the patient support apparatus 20 using one or more wireless access points 76. When the command is received at the patient support apparatus 20, controller 48 checks to see if the command is an authorized command and, if so, implements the command.

After both steps 163 and 165 of main algorithm 226 (FIG. 5), caregiver assistance application 124 proceeds to step 167 where it checks to see if the caregiver has manually input a command to change the currently displayed room overview screen 162 back to the room listing screen 156 of FIG. 7. If the caregiver has, algorithm 226 returns back to step 154 and proceeds in the manner previously described. If the caregiver has not, algorithm returns back to step 173 where it either continues to display the room listing screen or it automatically changes the screen to the room overview screen.

It should be noted that the display of different screens within caregiver assistance application 124 is not only controlled by the area that a user presses/selects on a particular screen, but also by the caregiver's use of the conventional “back” and “forward” functions of the web browser that the caregiver is using to access caregiver assistance application 124. Thus, for example, if a user is viewing room overview screen 162 of FIG. 8 and wishes to return to viewing room listing screen 156 of FIG. 7, he or she can simply press, or otherwise activate, the “back” function of the web browser the caregiver is using. These and other navigation features (e.g. a “home” icon, a menu icon, etc.) are not shown in FIG. 5 but are included in at least some embodiments of caregiver assistance application 124.

It should also be noted that main algorithm 226 may be modified substantially from what is shown in FIG. 5 and what has been described above. Such modifications include the addition of one or more steps or screens not described with respect to FIG. 5, as well as the removal of one or more steps and/or a changing of sequence of one or more steps. In one specific embodiment, main algorithm 226 omits the automatic screen selection feature of step 173. In another embodiment, main algorithm 226 includes one or more icons on one or more of the displayed screens that allow a user to perform the sharing and unsharing functions of algorithm 175. Still other modifications are possible.

During the execution of patient bed sore risk reduction algorithm 141, caregiver assistance application 124 may be programmed to automatically execute skin documentation algorithm 800, or it may be programmed to present an option to the caregiver to manually begin execution of the skin documentation algorithm 800. Regardless of how it is started, skin documentation algorithm 800 starts at step 802 where caregiver assistance application 124 determines which room or bed bay the patient is located in whose skin condition is to be reviewed and/or documented. In most instances, this information is already known from the room number and/or bed bay number displayed in room identifier location 198. Therefore, the caregiver doesn't have to do anything for step 802 unless the caregiver wishes to document a skin condition of a patient who is located in a room or bed bay different from the one that is currently identified on the electronic device 104 being used by the caregiver, or unless the caregiver starts algorithm 800 from a screen that doesn't have a room, bed bay, and/or patient identifier associated with it.

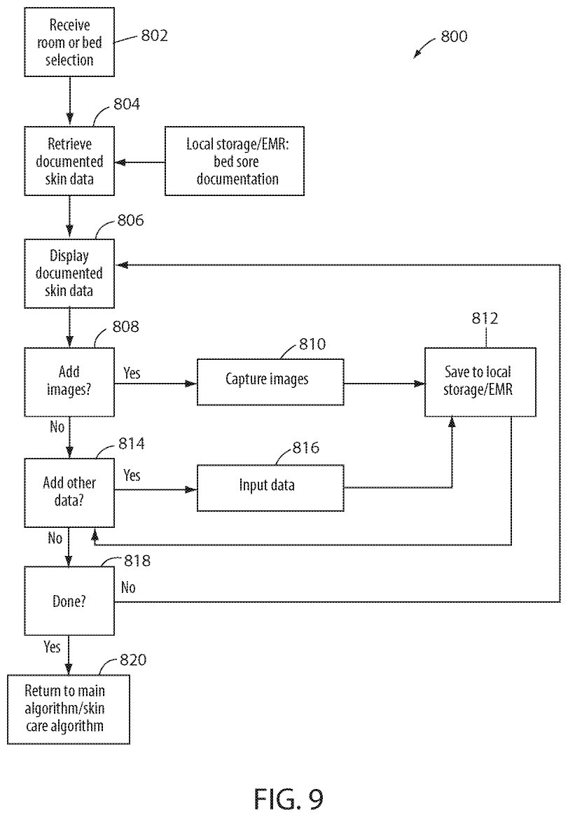

After step 802 of skin documentation algorithm 800 (FIG. 9), caregiver assistance application 124 proceeds to step 804 where it retrieves any skin documentation that has been previously saved for that particular patient. Such prior skin documentation may be saved both on EMR server 98 and within data repository 128, and caregiver assistance application 124 checks both locations and retrieves whatever skin documentation data is saved at either or both of these locations. After retrieving this data, application 124 proceeds to step 806 where it displays the retrieved skin care documentation. Depending upon the volume of data that has been previously documented, caregiver assistance application 124 may initially display only a portion of the data along with navigation tools (e.g. forward and backward arrows, page jump icons, etc.) that enable the caregiver to use electronic device 104 to efficiently view all of the documentation that has been previously been saved.

At step 808 (FIG. 9), caregiver assistance application 124 displays a prompt, indicator, or other selection control that enables the caregiver to choose whether he or she wishes to add any image data to the skin documentation. Such image data includes pictures taken of one or more locations of the patient's skin using a digital camera. If the caregiver wishes to add one or more of such images, he or she selects the image addition option and caregiver assistance application 124 proceeds to step 810. At step 810, the caregiver uses the built-in camera of his or her mobile electronic device 104a to capture one or more images of the patient's skin. After the caregiver has finished taking digital images, application 124 proceeds to step 812 where it saves the images to the patient's medical records by sending them to EMR server 98. In some embodiments, caregiver assistance application 124 may also save the images to data repository 128.

After completing step 812, caregiver assistance application 124 proceeds to step 814 where it inquires whether the caregiver wishes to add any other data regarding the patient′ skin conditions. Such other data may include measurements, notes, or other information that is not captured, or is otherwise not easily seen from, the image data captured at step 810. If the caregiver wishes to add such additional data, application 124 proceeds to step 816 where the caregiver enters the additional data. After step 816, application 124 returns to step 812 and sends the additional data to EMR server 98 and/or data repository 128.

After the caregiver has added image data and/or other data to the medical record of the patient at steps 810 and 816, caregiver assistance application 124 proceeds to step 818 where it inquires of the caregiver whether he or she is finished adding such data. If the caregiver is not finished, application 124 returns to step 806 and proceeds in the manner previously described. If the caregiver is finished, application 124 proceeds to step 820 where it returns back to executing main algorithm 226, bed sore risk reduction algorithm 141, and/or mattress control algorithm 700.

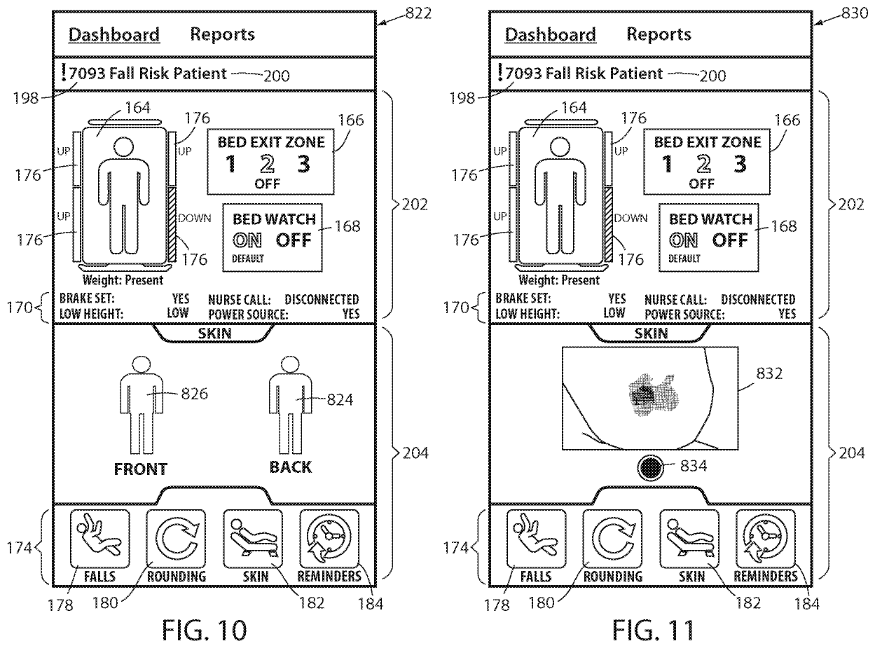

FIGS. 10-16 illustrate several examples of different screens that may be displayed by caregiver assistance application 124 on electronic devices 104 during the execution of skin documentation algorithm 800. It will be understood that these screens are merely illustrative examples of several types of screens that caregiver assistance application 124 may be configured to display. Additional screens may be displayed and/or fewer screens may be displayed. Further, the content of whatever screens are displayed may be modified from the examples shown in FIGS. 10-16. Finally, the reference to “first,” “second,” “third,” etc. with respect to the screens of FIGS. 10-16 is not meant to signify any sequential order to these screens, but instead is used merely to distinguish one screen from another.

FIG. 10 illustrates a first skin care data input screen 822. First skin care data input screen 822 is displayed by caregiver assistance application 124 during step 810 or 816 in order to document which side of the patient the skin data being entered corresponds to. If the caregiver wishes to enter data regarding a skin condition located on the back of the patient, the caregiver presses a back icon 824. If the caregiver wishes to enter data regarding a skin condition that is located on the front of the patient, he or she presses a front icon 826. Screen 822 may be modified to allow a user to specify the location of the skin condition with even greater granularity, or additional screens may be included for specifying this additional granularity. Such additional granularity may include icons for specifying that the skin condition is located on the patient's right or left leg, right or left arm, right or left hand, right or left foot, head, torso, and/or at other more specific locations. Once the user makes the desired selection, caregiver assistance application 124 associates the data that is subsequently added to the patient's medical record (or that was immediately previously added, in some embodiments) as corresponding to the selected location on the patient's body. Thus, the location selection function of screen 822 enables the caregiver to easily and graphically specify the location of the skin condition he or she is documenting, and this information is included as part of the data that is sent at step 812.

FIG. 11 illustrates a second skin care data input screen 830. Second skin care data input screen 830 is displayed by caregiver assistance application 124 during step 810 when the caregiver wishes to add image data to the patient's medical documentation. Screen 830 includes an image window 832 and a capture icon 834. The image window 832 shows the image that the built-in camera of the electronic device 104 is currently sensing. Image window 832 thus changes dynamically as the caregiver moves the electronic device 104 to different locations and/or orientations. The capture icon 834 is touched by the caregiver when the caregiver wishes to take and save a picture corresponding to the image currently shown in window 832. After the user touches the capture icon 834, the captured image is sent at step 812 to the EMR server 98 and/or data repository 128.

It will be understood that second skin care data input screen is only functional for those electronic devices 104 that include built-in cameras, which is typically the case for smart phones 104a, tablets 104a, and/or laptops 104a. Stationary electronic devices 104b and certain other mobile electronic devices 104a, however, might not include such a camera. For such electronic devices 104, screen 830 is either not functional or caregiver assistance application 124 may be configured to not display screen 830 at all (as well as any other screens associated with step 810).

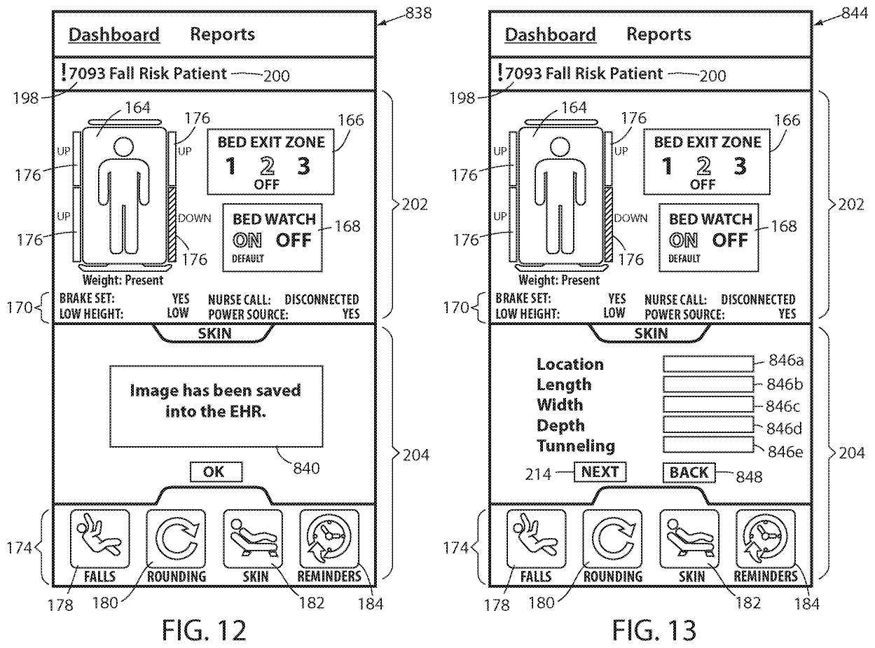

FIG. 12 illustrates a documentation confirmation screen 838 that is displayed by caregiver assistance application 124 after the caregiver has sent data to the EMR server 98 and/or data repository 128 at step 812 of algorithm 800. Documentation confirmation screen 838 includes an information window 840 in which caregiver assistance application 124 displays information regarding the status of the information sent to EMR server 98 and/or data repository 128. That is, once the data is sent at step 812, caregiver assistance application 124 awaits an acknowledgement from either or both recipients and, once received, displays information within window 840 confirming that the sent data was received and saved. If no acknowledgement is received, or there is another error in the transmission or receipt of the data, caregiver assistance application 124 displays other information about the error within window 840. Caregiver assistance application 124 therefore provides information to the caregiver that either confirms the documentation of skin data and/or lets the caregiver know if there was an error in the documentation process.

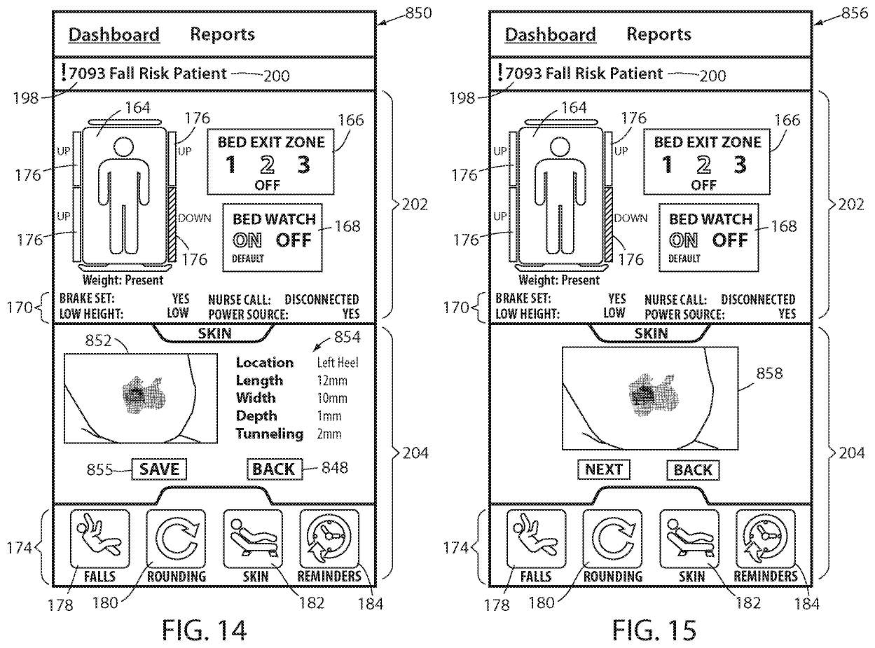

FIG. 13 illustrates a third skin care data input screen 844. Third skin care data input screen 844 is displayed on an electronic device 104 by caregiver assistance application 124 during the data input step 816. Third skin care data input screen 844 includes a plurality of data input windows 846a-e. Although the specific data input windows 846a-e shown in FIG. 13 are used by the caregiver to input data regarding an existing bed sore, it will be understood that other data input windows 846 may be displayed in association with screen 844 in order to input other data regarding one or more conditions of the patient's skin. In the specific example shown in FIG. 13, data input window 846a is used to specify the location of the patient's existing bed sore; data input window 846b is used to specify the length of the existing bed sore; data input window 846c is used to specify the width of the existing bed sore; data input window 846d is used to specify the depth of the bed sore; and data input window 846e is used to specify whether the bed sore is tunneling or not. In order to input data into these windows 846, the caregiver may use either the physical keypad of the electronic device 104 (if there is one) or a virtual keypad that is displayed on the screen (not shown) after the user presses on one of the windows 846. After the caregiver is finished inputting the desired data, the caregiver can select either the “next” icon 214 or a “back” icon 848. Touching “next” icon 214, in some embodiments, takes the caregiver to a screen like screen 850 of FIG. 14. Touching “back” icon 848, in some embodiments, takes the caregiver back to one of the previously described data input screens (e.g. screen 822 or 830).

FIG. 14 displays a fourth skin care data input screen 850. Fourth skin care data input screen 850 is displayed after a caregiver has entered at least one image and data regarding that image, the latter of which may be entered via screen 844 of FIG. 13. Fourth skin care data input screen 850 includes both a captured image window 852 and a data summary area 854. Capture image window 852 displays one of the images captured at step 810 using 2^{nd} data input screen 830 of FIG. 11. Data summary area 854 displays the data input as part of step 810 (or step 816) that relates to the image shown in window 852. This data may be input using third skin care data input screen 844 (FIG. 13). Screen 850 therefore displays both a captured image of the patient' skin, as well as information about that image. Screen 850 also includes a “save” icon 855 and “back” icon 848. The user touches “save” icon 856 when the user wishes to save the data shown in window 852 and summary area 854 to the EMR server 98 and/or data repository 128. Touching “save” icon 855 thus corresponds to step 812 of algorithm 800. If the user does not wish to save the data illustrated in screen 850, or wishes to make modifications to it before saving it, he or she may touch “back” icon 848, which returns the caregiver to one of the screens previously described and shown in FIGS. 10, 11, or 13, or still another screen.

FIG. 15 illustrates a saved images review screen 856 that is displayable by caregiver assistance application 124 after one or more images have been captured of the patient's skin condition. Screen 856 may be displayed as part of 810 (e.g. after one or more images have been captured at step 810). Screen 856 includes a saved image window 858 in which a previously captured image is displayed, as well as “next” icon 214 and “back” icon 848. Touching the “next” icon 214 causes caregiver assistance application 124 to display the next image in the set of captured images. Touching the “back” icon 848 causes caregiver assistance application 124 to display an earlier image in the set of captured images, or alternatively takes the caregiver back to another screen that was displayed prior to screen 856. Screen 856 may be further modified to allow a user to select one of the saved images in order to add additional data to it, such as the additional data which is input in FIG. 13. In one embodiment, touching and holding a particular image within window 858 causes caregiver assistance application 124 to display screen 844 of FIG. 13, thereby allowing the caregiver to enter additional data regarding the selected image. Screen 856 may be used in still other manners and/or display still other information.

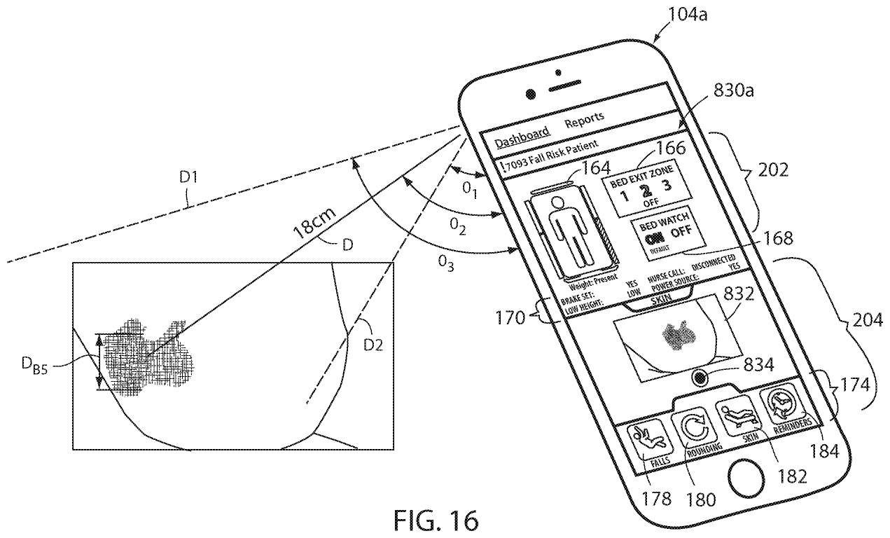

FIG. 16 illustrates an alternative image capture screen 830a that may be used with certain electronic devices 104a that are capable of automatically measuring the distances of objects whose images are captured by the device 104. That is, screen 830a may be used with smart phones, or other computer devices, that include software adapted to determine the physical distance of objects whose images are captured by the device's camera. Such automatic measurement programming may be incorporated into the electronic device 104 as a conventional feature of the electronic device, or it may be part of a separate app downloaded onto the device 104 that is utilized by caregiver assistance application 124, or it may be part of caregiver assistance application 124 itself.

The automatic measurement feature utilized with capture screen 830a typically involves the electronic device making distance measurements (e.g. through IR emissions or reflections) to the object that is being photographed. As shown in FIG. 16, electronic device 104 may determine a distance D between itself and a patient's bed sore. This distance may be determined at multiple positions on one or more landmarks of the object being photographs, as shown by distance measurements D1 and D2 in FIG. 16. From the distance measurements D, D1, D2, and, in some instances, the known angles at which the distance measurements are taken with respect to the electronic device 104 (e.g. angles Θ1, Θ2, and Θ3), caregiver assistance application a distance D_{bs}, which corresponds to a physical distance across at least one dimension of the bed sore shown in FIG. 16. The distance D_{bs} is then entered automatically by caregiver assistance application into one of data input windows 846b or 846c, thereby allowing the caregiver to avoid manually entering this information.

In some embodiments, electronic device 104 may be an iPhone sold by Apple Corporation, that includes the iOS12 update, or later, in which case the Apple ARKit technology is inherently included within the iPhone, and this technology allows a user to use the camera of the iPhone to take automatic distance measurements of the kind illustrated in FIG. 16. For Android based electronic devices 104, the distance measuring of FIG. 16 may be accomplished using an app downloaded from the Google Play store, such as, but not limited to, any one or more of the Ruler app from NixGames, the Smart Measure app by Smart Tools Co., the EasyMeasure app by Caramba Apps, and/or still other apps.

Screen 830a provides all of the same functionality of screen 830, but also adds the additional automatic measurement feature. That is, screen 830a includes a capture icon 834 that the caregiver touches when he or she wishes to take and save a picture corresponding to the image currently shown in window 832. After the user touches the capture icon 834, the captured image is sent at step 812 of algorithm 800 to the EMR server 98 and/or data repository 128.

It will be understood that screens 822, 830, 830a, 838, 844, 850, and/or 856 may be modified in a variety of different manners. For example, any one or more of these screens may include additional control icons allowing the caregiver to navigate from one of these screens to another. In such embodiments, when the caregiver is finished making his or her selections on a first one of these screens, he or she can easily navigate to another one of these screens if he or she wishes to input additional data or images, review previously input images or data, or edit previously input images or data. Once the caregiver has completed his or her data input, touching the save icon, such as “save” icon 855 of FIG. 14, causes caregiver assistance application 124 to send all of the input data to EMR server 98 and/or data repository 128. The sending of this data may also involve time stamping the data when it is sent, and/or caregiver assistance application 124 may automatically time stamps the input data at the moment it is input or captured. Still other modifications are possible.

In some embodiments, mobile electronic device 104a may be configured to automatically delete any such image data from its memory after it has been transmitted to caregiver assistance application 124 (and/or uploaded to an electronic medical records server). In some cases, the digital image may be retained on mobile electronic device 104a until it receives an acknowledgement of the receipt of the digital image by caregiver assistance application 124 and/or by the EMR. In still other embodiments, mobile electronic device 104a may be configured to automatically delete the digital image from its memory after either an acknowledgement receipt is received or a predetermined amount of time passes since the digital image was transmitted, whichever is sooner.

In some embodiments, any time mobile electronic device 104a sends a digital image, or other patient data, it is configured to do so without transmitting any information that uniquely identifies the patient associated with that image or data. Mobile electronic device 104a is able to send digital images and other data regarding a patient to caregiver assistance application 124 without any patient identification information because the data sent by mobile electronic device 104a is correlated with either a particular patient support apparatus 20, 20a, or a particular room (or bay within the room), not with a particular patient. Caregiver assistance application 124 is able to associate the data with a particular patient, if needed, because it maintains access to data tables that correlate specific patients to specific rooms, room bays, and/or patient support apparatuses. Electronic devices 104 therefore can operate without storing any patient identification data in their memories.

FIG. 17 illustrates one embodiment of a screen selection algorithm 139 that may be executed by caregiver assistance application 124. As mentioned previously, screen selection algorithm 139 is executed at step 173 of main algorithm 226 (FIG. 5), in at least one embodiment of caregiver assistance application 124. Screen selection algorithm 139 is only executed by caregiver assistance application 124 for mobile electronic devices 104a, and not for stationary electronic devices 104b. Screen selection algorithm 139 begins at step 660 where caregiver assistance application 124 determines the set of patients to which the particular caregiver using caregiver assistance application 124 is assigned. In other words, caregiver assistance application 124 executes algorithm 139 for each individual caregiver and automatically changes the screens displayed on each of the mobile electronic devices 104a for each of the individual caregivers. Thus, at step 660, caregiver assistance application 124 identifies which caregiver it is executing algorithm 139 with respect to, and then identifies the patients assigned to that particular caregiver.

After determining the caregiver's assigned set of patients at step 660, caregiver assistance application 124 proceeds to step 662 where it determines if the caregiver's mobile electronic device 104b has detected any short range signals from a patient support apparatus 20. As described below with respect to caregiver assistance systems 106a, 106b, and/or 106c, patient support apparatuses 20 may be modified to include a short range transceiver 320 that sends out short range signals that are detectable by mobile electronic devices 104a when the mobile electronic device 104a is positioned in proximity to such a modified patient support apparatus 20. Such proximity may refer to being in the same room as, or positioned next to, the particular patient support apparatus 20 that is equipped with such a short range transceiver 320. Step 662 therefore is only executed in those embodiments of caregiver assistance system 106 in which at least one patient support apparatus 20 is equipped with such a short range transceiver 320.

If the mobile electronic device 104a detects a short range signal from a patient support apparatus 20 at step 662, it notifies caregiver assistance application 124 and proceeds to step 664. At step 664, caregiver assistance application 124 checks to see if the short range signal received at step 662 was from a patient support apparatus 20 whose patient is one of the patient's on the caregiver's assigned set of patients. This is accomplished, in at least some embodiments, by configuring patient support apparatuses 20 to send out a unique identifier (e.g. identifier 186) via the short range transceiver 320 that uniquely identifies that particular patient support apparatus 20. When/if mobile electronic device 104a detects this short range signal, it is evidence that mobile electronic device 104a is currently collocated in the same room, or same bay area of a room, as the patient support apparatus 20 whose short range signal it detected. This is because, due to the short range of the signal, the mobile electronic device 104a is only able to detect that signal when it is in close proximity to the patient support apparatus.

The unique patient support apparatus identifier that mobile electronic device 104a receives via the short range signal is forwarded by the mobile electronic device 104a to caregiver assistance application 124 upon receipt, or shortly thereafter, by mobile electronic device 104a. The fact that mobile electronic device 104a forwards this information upon receipt, or shortly thereafter (e.g. within several seconds and up to a minute or so), along with the fact that the unique identifier is only detected when mobile electronic device 104a is in close proximity to patient support apparatus 20, indicates to caregiver assistance application 124 that the mobile electronic device 104a should currently be associated with the particular patient support apparatus 20 whose unique identifier mobile electronic device 104a just received and forwarded to caregiver assistance application 124. In other words, the combination of the unique patient support apparatus identifier forwarded by the mobile electronic device 104a to caregiver assistance application 124 with the fact that this identifier is forwarded upon receipt, or shortly thereafter, serve as correlation data allowing caregiver assistance application 124 to temporarily correlate that particular mobile electronic device 104a with a particular patient support apparatus 20 and/or a particular patient.

Returning back to algorithm 139 (FIG. 17), caregiver assistance application 124 uses the unique patient support apparatus identifier at step 664 to determine if that particular patient support apparatus 20 has been assigned to a patient who is part of the patient assignment set for the caregiver associated with that particular mobile electronic device 104a. In other words, caregiver assistance application 124 determines if the patient onboard that particular patient support apparatus 20 is one of the caregiver's patients, or if the patient is another caregiver's patient. If the patient is the responsibility of another caregiver, caregiver assistance application 124 proceeds to step 670. If the patient is one of the caregiver's patients, caregiver assistance application 124 proceeds to step 668.

At step 668 (FIG. 17), caregiver assistance application 124 automatically displays the room overview screen corresponding to the patient support apparatus 20 whose short range signal was received at step 662. As described previously, FIG. 8 illustrates an example of a room overview screen 162. Other types of room overview screens may alternatively be used. The effect of steps 662, 664, and 668 is that, if a caregiver walks into a room in which one of his or her assigned patients is located, caregiver assistance application 124 will automatically display a room overview screen (162, 162a) on his or her mobile electronic device 104a that corresponds to the room in which he or she just entered (assuming the patient support apparatus 20 is configured with a short range transceiver 320). Further, if the caregiver walks into a room that contains a patient support apparatus 20 having a short range transceiver 320, but the patient support apparatus 20 is assigned to a different caregiver, caregiver assistance application 124 will not display the room overview screen for that room because, as determined at step 664, the patient in that room is not assigned to that particular caregiver. Still further, when the caregiver exits the room, the short range signal will no longer be detected and algorithm 175 will therefore switch to displaying a room listing screen when the caregiver is in the hallway (or anywhere else outside of an assigned patient's room), as will be discussed further below.

Returning to step 662 of screen selection algorithm 139 (FIG. 17), if the caregiver's mobile electronic device 104 a does not detect any short range signals from a patient support apparatus 20 at step 662, it proceeds to step 666 where it checks to see if any images of a particular patient support apparatus 20 have been captured. Such images are captured using the camera app onboard the mobile electronic device 104a. Such images include, but are not limited to, images that uniquely identify a particular patient support apparatus 20. For example, such images include a bar code, a QR code, or some other visual identifier that is either permanently displayed somewhere on the patient support apparatus 20, or that is selectively displayed on display 70 of the patient support apparatus 20. In some embodiments, the capture of these images are time stamped by mobile electronic device 104a and/or caregiver assistance application 124, and the image and time stamp are used a correlation data that indicates that, at the time of the image capture, the mobile electronic device 104a was collocated with the particular patient support apparatus 20 whose visual identifier is shown in the captured image.

If the user captures an image of such a visual identifier, caregiver assistance application 124 proceeds to step 664 where it analyzes the image. The analysis of the image is carried out at step 664 to determine if that particular patient support apparatus 20 corresponds to a patient to whom the caregiver is assigned. That is, step 664 is carried out in the same manner previously described except, instead of utilizing identifier 186 received via short range transceiver 320, caregiver assistance application 124 uses a visual identifier (which may visually identify identifier 186) to determine if the patient support apparatus 20 has been assigned to one of the caregiver's patients. If it has been, caregiver assistance application 124 proceeds to step 668 where it displays the corresponding room overview screen for that particular room. If it has not, caregiver assistance application 124 proceeds to step 670.

The effect of steps 666, 664, and 668 is to allow the caregiver to control what room overview screen 162, 162a is displayed on his or her mobile electronic device 104a by taking a picture of a visually unique identifier on a particular patient support apparatus 20. If the unique visual identifier corresponds to a patient support apparatus 20 for one of his or her assigned patients, then application 124 automatically displays the room overview screen 162 for that particular room. If it does not, application 124 proceeds to step 670.

At step 670 of algorithm 139 (FIG. 17), caregiver assistance application 124 checks to see if it has received either a manual selection of a room number and/or, in some embodiments, an audio (voice) selection of a particular room number. The manual selection may occur in different manners, such as, but not limited to, the caregiver typing in a particular room number using a keypad (physical or virtual) onboard his or her mobile electronic device 104a, the caregiver selecting a particular room from amongst those listed on a room listing view screen (e.g. screen 156 of FIG. 7), or by other means. In some embodiments, caregiver assistance application 124 includes, or otherwise utilizes, voice recognition software that enables the caregiver to speak a room number (or other room identifier) into the microphone onboard his or her mobile electronic device 104a and then processes the audio signals to determine a corresponding room number (or other room identifier). Caregiver assistance application 124 determines at step 670 whether any of these methods of selecting a particular room have occurred. If one has occurred, it proceeds to step 676. If none have occurred, it proceeds to step 672.

Turning first to step 672, caregiver assistance application 124 determines the current location of the caregiver at step 672. Step 672 is only included in those embodiments of caregiver assistance application 124 that utilize a real time locating server 100 (FIG. 4). As was described previously, real time location server 100 may be a conventional server that keeps track of the current locations of caregivers using one or more badges, beacons, and/or other devices. Regardless of the specific manner in which real time locating server 100 keeps track of caregiver locations, caregiver assistance application 124 sends a request for the caregiver's current location at step 672 of algorithm 139. Upon receiving a reply to this request, caregiver assistance application 124 proceeds to step 674. At step 674, it determines if the caregiver is currently located in a room, or outside of a room (e.g. a hallway, a nurses'station, etc.) If the caregiver is not located within a room, caregiver assistance application 124 proceeds to step 680 where it automatically displays a room listing screen, such as room listing screen 156 shown in FIG. 7.

If the real time locating server 100 indicates that the caregiver's current location is within a room (as determined at step 674 of algorithm 139; FIG. 17), caregiver assistance application 124 proceeds from step 674 to step 676. At step 676, caregiver assistance application 124 determines if the room in which the caregiver is currently located corresponds to a room in which one of his or her assigned patients is located. If it does, caregiver assistance application 124 displays a room overview screen for that particular room on the caregiver's mobile electronic device 104a. If it does not, caregiver assistance application 124 proceeds to step 680 where it displays a room listing screen on the caregiver's mobile electronic device 104a.

Returning back to step 670 of screen selection algorithm 139 (FIG. 17), caregiver assistance application 124 proceeds from step 670 to step 676 if the caregiver manually selects a room number, either aurally or by physically touching the screen (or other controls) on his or her mobile electronic device 104a. At step 676, caregiver assistance application 124 checks to see if the room manually entered by the caregiver at step 670 corresponds to a room in which one of his or her assigned patients is located. If it does, caregiver assistance application 124 proceeds to step 678 where it displays the room overview screen for that particular room. If it does not, caregiver assistance application 124 proceeds to step 680 where it displays the room listing screen for that particular caregiver.

The effect of all of the steps of screen selection algorithm 139 is that caregiver assistance application 124 will display the room overview screen of a patient assigned to that particular caregiver whenever the caregiver enters the room of that patient. This will be done either automatically (via step 662, 666, or 672) or manually (via step 670). Further, in those embodiments in which caregiver assistance application 124 communicates with real time locating server 100 and/or patient support apparatuses 20 include a short range transceiver 320, caregiver assistance application 124 will automatically switch to displaying a room listing screen whenever the caregiver leaves the room, or otherwise is positioned outside of a patient's room. In these embodiments, the caregiver is therefore automatically presented with a screen listing all of the rooms he or she is responsible for when he or she is positioned outside of a room, and automatically presented with the room overview screen when he or she enters a room.

An additional effect of screen selection algorithm 139 is that any data that the caregiver inputs into mobile electronic device 104a after he or she enters a room of one of his or her assigned patients is that such data will automatically be correlated to the correct patient and/or patient support apparatus 20 by caregiver assistance application 124. Thus, for example, if the caregiver inputs data into mobile electronic device 104a regarding the patient's skin condition (e.g. captures an image of a bed sore), caregiver assistance application 124 will automatically associate this image with the correct patient support apparatus 20 and correct patient due to the data being input while the mobile electronic device 104a is collocated with that particular patient and/or patient support apparatus 20. The same is true for other types of data entered by the caregiver, such as, but not limited to, a service request, fall-risk data, rounding data, etc. The caregiver therefore doesn't have manually identify either the particular patient or the particular patient support apparatus 20 to which the entered data applies. Instead, this is handled automatically by caregiver assistance application 124.

It will be understood that screen selection algorithm 139 (FIG. 17) is configured in some embodiments to not change whatever screen is being displayed on mobile electronic device 104a if no room selection inputs are received at steps 662, 666, 670, or 672. In such embodiments, the screen that caregiver assistance application 124 displays is only changed automatically if the caregiver's location changes from being inside a room to being outside a room, or vice versa.

Still further, caregiver may also pause for a predetermined period of time before making any automatic change to the screen displayed on mobile electronic device 104a. During the pause, caregiver assistance application 124 monitors mobile electronic device 104a for any inputs by the caregiver. If any inputs are detected, the automatic screen switch is delayed or cancelled. If no user inputs are detected during the pause period, then caregiver assistance application 124 automatically switches the screen after the pause period time has expired. The inclusion of such a pause period helps reduce or eliminate interrupting the displayed screen while the caregiver is using caregiver assistance application 124. In other words, caregiver assistance application 124 only changes the screen automatically if the mobile electronic device 104a has been idle for predetermined amount of time.

In another modified embodiment of screen selection algorithm 139, caregiver assistance application 124 may be configured to prompt the caregiver to allow the change of the displayed screen before making such a change. The prompt may include a popup window, or some other message, that asks the caregiver for permission to change the displayed screen. In such embodiments, only if the user accepts the screen change (or, in some embodiments, if no response is received within a predetermined time period), does caregiver assistance application 124 automatically change the screen displayed on the corresponding mobile electronic device 104a.

It will be understood that still other modifications may be made to screen selection algorithm 139. Such modifications include, but are not limited to, omitting step 662 completely (particularly if patient support apparatuses 20 do not include a short range transceiver 320), omitting step 666 (particularly if patient support apparatuses 20 are not configured to visually display a unique identifier), and/or omitting step 672 completely if caregiver assistance application 124 is not configured to communicate with a real time locating server 100 (or the particular healthcare facility does not have a real time locating server 100). Still further, it will be understood that the order in which the steps of algorithm 139 are executed may be varied from what is shown in FIG. 17.

It will be understood that in all of the embodiments of screen selection algorithm 139, the screen automatically selected by algorithm 139 is only the initial screen that is initially displayed when the caregiver enters a room (or selects a particular patient support apparatus) and/or leaves a room. That is, the caregiver is still free to select different screens after caregiver assistance application 124 has initially selected a room overview screen or a room listing screen at steps 668, 678, and/or 680. Thus, for example, if a caregiver initially walks into a patient's room and caregiver assistance application 124 initially displays a room overview screen for that particular room, the caregiver is free to press on any of the task icons 178, 180, 182, and/or 184 to bring up other screens for carrying out these tasks, or to manually change the room overview screen to a room listing screen (or vice versa), or to make still other manual changes to the screen displayed. Algorithm 139 is therefore included to help reduce the amount of work required by the caregiver in selecting a particular initial screen, but not to limit the screens that are available to the caregiver.

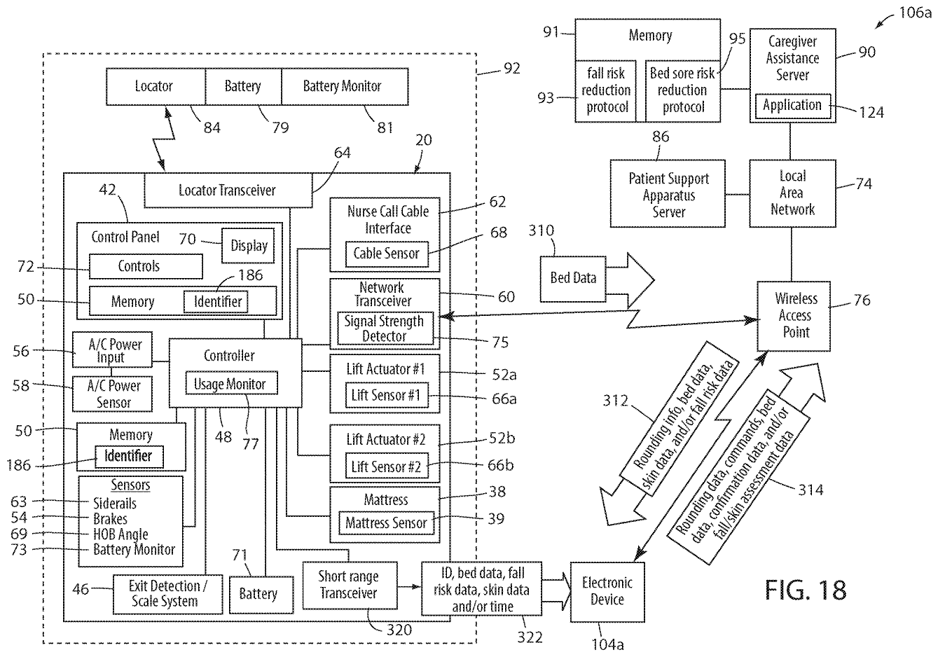

It will also be understood that caregiver assistance application 124 is configured, in some embodiments, to automatically utilize the short range signal received by electronic device 106 from short range transmitter 320 (FIG. 18) and forwarded to application 124, and/or the real time location signal received as part of algorithm 139, to implement an automatic alert silencing feature. The automatic alert silencing feature operates to automatically silence, or reduce the volume of, any patient support apparatus audio alerts in response to a caregiver entering the same room as the patient support apparatus 20 that is emitting the alert. In some embodiments, this feature only serves to temporarily silence and/or reduce the volume of such alerts. That is, if the caregiver does not manually shut off the alert using one or more of the control panels on the patient support apparatus 20, the patient support apparatus 20 will begin emitting the alert (or increase the volume of the alert back to its normal volume) automatically upon the caregiver leaving the room.

The automatic alert silencing feature operates in the following manner. Whenever a patient support apparatus 20 emits an alert, it communicates this information to caregiver assistance application 124. Whenever the patient support apparatus 20 stops emitting the alert, it also communicates this information to caregiver assistance application 124. Whenever caregiver assistance application 124 receives information from a mobile electronic device 104a that that mobile electronic device 104a is currently collocated in the same room as a patient support apparatus that is currently emitting an alert, caregiver assistance application 124 sends a command (via wireless access point 76 and network transceiver 60) to that particular patient support apparatus instructing it to either silence or reduce the volume of its audible alert. Similarly, if the caregiver does not manually disable the alert, or otherwise rectify its cause while he or she is in the room, caregiver assistance application 124 is configured to send—when it detects the caregiver is no longer collocated in the same room—another command to that patient support apparatus instructing it to un-silence, or increase the volume of, its alarm.

In some embodiments, the automatic alert silencing feature is configured to only silence, or reduce the volume of, the audible portion of a patient support apparatus alert. Thus, for example, if the patient support apparatus 20, 20a is also configured to flash one or more lights, display a particular screen on its display, or otherwise take one or more actions that change a visual characteristic of the patient support apparatus 20, caregiver assistance application 124 is configured to not instruct the patient support apparatus 20 to stop producing these visual changes when a caregiver walks into the same room as that patient support apparatus. In still other embodiments, caregiver assistance application 124 is configurable by authorized personnel to choose whether the automatic alert silencing feature applies to both the visual and audible portions of an alert, or to only the audible portion.

A variety of structural modifications may be made to caregiver assistance system 106 beyond those previously discussed herein. For example, although caregiver assistance system 106 has been described herein as utilizing a caregiver assistance application 124 executed on caregiver assistance server 90 and accessed by electronic devices 104 having conventional web-browser applications stored thereon, caregiver assistance system 106 may be modified to include one or more native applications that execute on the electronic devices 104a or b themselves. In some of these modified embodiments, the caregiver does not need to open up the web-browser to access caregiver assistance application 124, but instead opens up a local caregiver assistance software application on the electronic device 104 that interacts with the caregiver assistance application 124 being executed on caregiver assistance server 90. In such embodiments, it may be easier to provide alerts to the caregiver by having the electronic device vibrate, emit an audible sound, and/or illuminate one or more lights on the device. Such alerts may be more difficult to communicate to a caregiver when caregiver assistance system 106 is implemented using browser-connected electronic devices 104, particularly if the caregiver has the browser application closed and/or running in the background and/or is not looking at the information currently being displayed on the screen of the electronic device 104. Such native applications may be programmed for execution with the Android or iOS operating systems, or still other operating systems utilized by the electronic device 104.

It will be understood by those skilled in the art that, although caregiver assistance application 124 has been primarily described herein with reference to a single caregiver using a single electronic device 104, caregiver assistance application 124 is not limited to use by only a single caregiver and/or a single electronic device 104. Further, caregiver assistance application 124 is not limited to use with only a single patient support apparatus 20 or a single patient. Instead, caregiver assistance application 124 is configured to be used, if desired, with all of the patient support apparatuses 20 within the healthcare facility, as well any or all of the caregivers within the healthcare facility. Such use of caregiver assistance application 124 by multiple caregivers can occur simultaneously. That is, multiple caregivers may be logged into caregiver assistance application 124 at the same time. In such cases, caregiver assistance application 124 is configured to display the room, patient, and/or patient support apparatus information discussed above for the set of rooms, patients, and/or patient support apparatuses 20 assigned to that particular caregiver. In other words, each caregiver (other than those with administrative access) is only able to view the room, patient, and patient support apparatus information for the rooms and/or patients assigned to that particular caregiver. Unless otherwise configured by an authorized individual, alerts associated with those patients, rooms, and/or patient support apparatuses 20 are only communicated by caregiver assistance application 124 to the mobile electronic device 104a associated with that caregiver (and, in some cases, to the stationary electronic device 104b that is associated with that particular room or patient).

Stationary electronic devices 104b are typically not used to perform rounding tasks and/or patient risk assessments because they cannot be carried with the caregiver to a patient's room, and thus are difficult to use for capturing images or assessment information and/or performing other tasks in the patient's presence. Nevertheless, stationary electronic devices 104b are capable of displaying all of the screens previously described and associated with caregiver assistance application 124, and receiving all of the data that is input on these screens, including not only answers to rounding and/or assessment questions, but also commands to change components on the patient support apparatuses 20. Further, authorized individuals 136 can configure caregiver assistance application 124 as they see fit with respect to what, if any, alerts are displayed on the stationary electronic devices 104b. For example, if a particular stationary electronic device 104b is associated with a particular wing of the healthcare facility, then the authorized individual 136 may configure caregiver assistance application 124 to notify the stationary electronic device 104b whenever any alert from any room or patient support apparatus 20 within that wing is issued. This can be configured even if the different rooms and/or patient support apparatuses 20 are assigned to different caregivers. As a result, caregiver A may receive alerts on his or her mobile electronic device 104a for a first set of rooms in that particular wing; caregiver B may receive alerts on his or her mobile electronic device 104a for a second set of rooms in that particular wing; and the stationary electronic device 104b associated with that wing may receive alerts for both the first and the second sets of rooms (and any other rooms in that particular wing). Still other variations are possible.

The data flows of caregiver assistance system 106 between caregiver assistance server 90, patient support apparatuses 20, and electronic devices 104 are illustrated in greater detail in FIG. 2. As shown therein, patient support apparatuses 20 transmit patient support apparatus messages 310 to patient support apparatus server 86 (or directly to caregiver assistance server 90) via network transceivers 60 and wireless access points 76. The patient support apparatus data contained within messages 310 includes such things as the status of the exit detection system 46 (armed or disarmed), the status of the siderails 36 (up or down), the status of the electrical power cord 102 (plugged in or not), the status of the nurse call cable 78 (plugged in or not), the status of the brake (on or off), the height of the litter frame 28, the status of mattress 38 (including any current therapy protocols being implemented using mattress 38), the status of the bed monitoring system, any existing alerts, and/or other data about patient support apparatus 20.

Caregiver assistance server 90, after receiving the data in these messages, transmits outbound messages 312 to selected ones of the electronic device 104 (FIG. 2). The content of the outbound messages 312 includes all or selected portions of the patient support apparatus data received via messages 310. Most of this patient support apparatus data is displayed on the screens in top portion 202. The outbound messages 312 also include the data content for the display screens shown as part of main algorithm 226, rounding algorithm 140, skin care algorithm 141, fall risk reduction algorithm 143, skin documentation algorithm 800, and other algorithms.

Caregiver assistance server 90 receives inbound message 314 from the electronic devices 104 in which it is in communication (FIG. 2). Inbound messages 314 include rounding data, patient support apparatus commands, fall-risk and/or bed sore-risk assessment data, skin documentation data and/or other documentation data, service information (e.g. service requests), verification data, and/or other data. The rounding data includes the answers and/or acknowledgements corresponding to the rounding questions displayed on first through fourth rounding screens 190, 220, 230, and 240, and the fall-risk assessment data includes the answers to the fall risk questions that are asked as part of algorithm 143. The bed sore risk assessment data includes any of the data entered by the caregiver using the various screens associated with the bed sore risk reduction algorithm 141, including the documentation algorithm 800. The patient support apparatus commands include any commands input by the caregiver into the electronic device 104 to change a state of the corresponding patient support apparatus 20. The skin documentation data includes any data (image and/or textual data) regarding the patient's skin condition, and the service data includes any data regarding the servicing of a patient support apparatus 20. As discussed previously, such commands include commands to arm exit detection system 46 and/or commands to arm a bed monitoring system, as well as other commands.

Inbound messages 314 may also include verification data, which is data gathered by mobile electronic device 104a that verifies the actual physical presence of the caregiver adjacent the patient support apparatus whose patient the caregiver is performing rounding duties for. More specifically, the verification data includes the images of a QR code, bar code, patient support apparatus, and/or caregivers that may be captured by the mobile electronic device 104a and sent to caregiver assistance application 124 as part of rounding algorithm 140.

It will be understood that the data flows illustrated in FIG. 2 may be modified significantly. For example, FIG. 18 illustrates a caregiver assistance system 106a according to another embodiment of the present disclosure. Caregiver assistance system 106a differs from caregiver assistance system 106 of FIG. 2 in that caregiver assistance system 106a includes different flows of messages sent between the caregiver assistance server 90, the mobile electronic devices 104a, and the patient support apparatuses 20. Caregiver assistance system 106a also differs from caregiver assistance system 106 of FIG. 2 in that it includes modified patient support apparatuses 20a that, unlike patient support apparatuses 20, include a short range transceiver 320. Further aspects of caregiver assistance system 106a are described below.

Patient support apparatuses 20a of caregiver assistance system 106a include all of the same components of patient support apparatuses 20 of caregiver assistance system 106. Those common components have been labeled with common numbers in FIG. 2 and, unless explicitly stated to the contrary below, the description of those components previously made above is equally applicable to these components. Caregiver assistance system 106a differs from caregiver assistance system 106 primarily in the source of various data (such as verification data, skin care data, fall risk assessment data, etc.) that is sent by electronic device 104 to caregiver assistance server 90 for use with rounding algorithm 140 and/or other algorithms. In some embodiments, this altered data flow enables a control panel on the patient support apparatus 20 to receive any of the data input by the caregiver into mobile electronic device 104a to be input into a control panel 42 on the patient support apparatus 20. The patient support apparatus 20 can then send the input data to mobile electronic device 104a for forwarding to server 90, or it may send it to server 90 via its direction connection with the wireless access points 76. In some cases, system 106a is implemented such that rounding verification data comes not from any images captured by electronic device 104, but instead from the short range transceiver 320 that is built into patient support apparatus 20a.

Short range transceiver 320 (FIG. 18) is adapted to wirelessly communicate with electronic devices 104 over a relatively short range. The short range is, in some embodiments, no larger than the typical size of a healthcare facility room such that, when a caregiver leaves a particular room, the caregiver's mobile electronic device 104a is no longer within range of the short range transceiver 320, and therefore no longer able to communicate with the short range transceiver 320. In some embodiments, short range transceiver 320 is an infrared transceiver adapted to communicate in line-of-sight situations with a corresponding infrared transceiver built into the mobile electronic device 104a. In other embodiments, short range transceiver 320 is a near field transceiver adapted to communicate with a near field transceiver built into mobile electronic device 104a. In still other embodiments, short range transceiver 320 is an RF transceiver having a relatively small power output such that communications are limited to within a short range of patient support apparatus 20a. Such RF transceivers may include, but are not limited to, Bluetooth transceivers. Still further, in some embodiments, short range transceiver 320 is an ultrasonic transceiver adapted to emit ultrasonic waves over a relatively short distance (e.g. generally within a room or bay area of a room). Such ultrasonic transmissions may use frequency modulation, amplitude modulation, phase modulation, and/or other techniques for communicating the unique identifier of the patient support apparatus 20. These transmissions are detected by a mobile electronic device 104a when it is collocated in the same room as the patient support apparatus 20.

Regardless of the specific short range transceiver 320 utilized by patient support apparatus 20a, controller 48 of patient support apparatus 20a is configured to transmit one or more patient support apparatus messages 322 using transceiver 320 to a nearby mobile electronic device 104a (FIG. 18). The messages 322 contain one or more of the following pieces of information: the unique identifier 186 of the corresponding patient support apparatus 20a; the current time; and/or sufficient patient support apparatus data to indicate whether the current status of the patient support apparatus 20 is in compliance with its desired settings or not. This information is transmitted periodically and repetitively in some embodiments of patient support apparatus 20a. In other embodiments, this information is transmitted only in response to an interrogation signal received from a mobile electronic device 104a. In still other embodiments, this information may be transmitted both repetitively and in response to interrogation signals.

Mobile electronic device 104a receives message(s) 322 when it is positioned within the vicinity of patient support apparatus 20a (FIG. 18). Mobile electronic device 104a uses the message 322 for carrying out the verification and/or compliance steps of rounding algorithm 140, for carrying out one or more aspects of bed sore risk reduction algorithm 141, for carrying out one or more aspects of the fall risk reduction algorithm 143, for carrying out one or more aspects of the documentation algorithm 800, for carrying out one or more aspects of the alarm silence feature, and/or for carrying out other operations in which the mobile electronic device 104a is to be correlated with a particular patient support apparatus 20 and/or patient.

With respect to patient rounding, in some embodiments, messages 322 are sent and captured by mobile electronic device 104a as part of algorithm 140. Mobile electronic device 104a uses the messages 322, particularly the patient support apparatus ID and/or time, to verify that it was physically present adjacent patient support apparatus 20a (i.e. collocated with patient support apparatus 20a) when the rounding occurred. This verification is handled, in some embodiments, internally via the programming of caregiver assistance application 124 such that the caregiver does not need to enter any information, or take any manual steps (other than positioning mobile electronic device 104a within range of transceiver 320) for this verification data to be received by mobile electronic device 104a and forwarded to caregiver assistance application 124. In other embodiments, in order to prevent a user (or electronic device 104a) from modifying the data contained within messages 322, the data is encrypted with an encryption algorithm that caregiver assistance application 124 is able to decrypt, but not mobile electronic device 104a. In still other embodiments, patient support apparatus 20a may be further modified to send a second message to caregiver assistance application 124 via network transceiver 60 whenever it transmits message 322 via short range transceiver 320. This second message confirms to caregiver assistance application 124 that message 322 was sent and, in some embodiments, contains the same information. If caregiver assistance application 124 does not receive this second message, it does not accept the verification data sent from mobile electronic device 104a.

With respect to bed sore risk reduction algorithm 141 and fall risk reduction algorithm 143, patient support apparatus messages 322 may identify the particular patient support apparatus 20 to mobile electronic device 104a (and thus caregiver assistance application 124) that the caregiver is currently positioned next to. This allows caregiver assistance application 124 to automatically, in at least some embodiments, bring up a screen that corresponds to that particular patient support apparatus 20 and the patient assigned thereto. This automatic screen selection may be part of the screen selection algorithm 139 described previously. If the caregiver wishes to perform a risk assessment (bed sore and/or fall) for a particular patient, he or she merely needs to walk within range of messages 322 and press the fall task icon 178. In response to pressing fall task icon 178, caregiver assistance application 124 automatically displays the appropriate screen with full knowledge of which patient (and/or which patient support apparatus 20) the risk assessment pertains to. The caregiver therefore is relieved of the task of manually identifying a specific room or a specific patient before proceeding to the risk assessment process of algorithms 141 and/or 143, as well as before proceeding to other tasks that involve data that is to be associated with a specific patient and/or patient support apparatus 20. Instead, caregiver assistance application 124 uses the specific patient support apparatus identifier 186 received within message 322 to determine which patient the subsequent risk assessment or other data applies to. Risk reduction algorithms 141 and/or 43 may also use data from messages 322 for other aspects.

Regardless of whether they are used by rounding algorithm 140, bed sore risk reduction algorithm 141, fall risk reduction algorithm 143, or another algorithm, messages 322 (FIG. 18) also include patient support apparatus data. In some embodiments, the patient support apparatus data only includes an indicator indicating whether the patient support apparatus 20 is in a compliant or non-compliant state. In other embodiments, the patient support apparatus data includes actual data about the state of each of the components of the patient support apparatus 20 and the determination of whether the patient support apparatus is in a compliant or non-compliant state is made by caregiver assistance application 124 based on the data communicated in message 322, as well as data stored in rules repository 126 defining the criteria for compliance. In either embodiment, the patient support apparatus data sent in message 322 is used by caregiver rounding algorithm 140, bed sore reduction algorithm 141, and/or fall risk reduction algorithm 143.

In some embodiments, message 322 may also include the current time. If included, this time information is also forwarded to caregiver assistance application 124. Caregiver assistance application 124 uses this time information to confirm the time that the caregiver was actually present at the patient's bedside when a rounding or other task was completed (or when data was entered). This time information is sent to EMR server 98 in some embodiments so that the time at which the rounding task, or other task, is recorded in the patient's electronic medical record. In other embodiments, patient support apparatus 20 may skip transmitting a time in message 322 and mobile electronic device 104a may append a time of receipt of message 322 in the data it sends to caregiver assistance application 124. As yet another alternative, both patient support apparatus 20 and mobile electronic device 104a may omit sending any time information and caregiver assistance application 124 can instead record the time at which it receives the inbound messages 314 from mobile electronic device 104a. In any of these embodiments (which may be wholly or partially combined), the time is used by caregiver assistance application 124 to determine and/or record when the caregiver completed his or her rounding task (or other task) for the particular patient assigned to the patient support apparatus 20 that sent message 322.

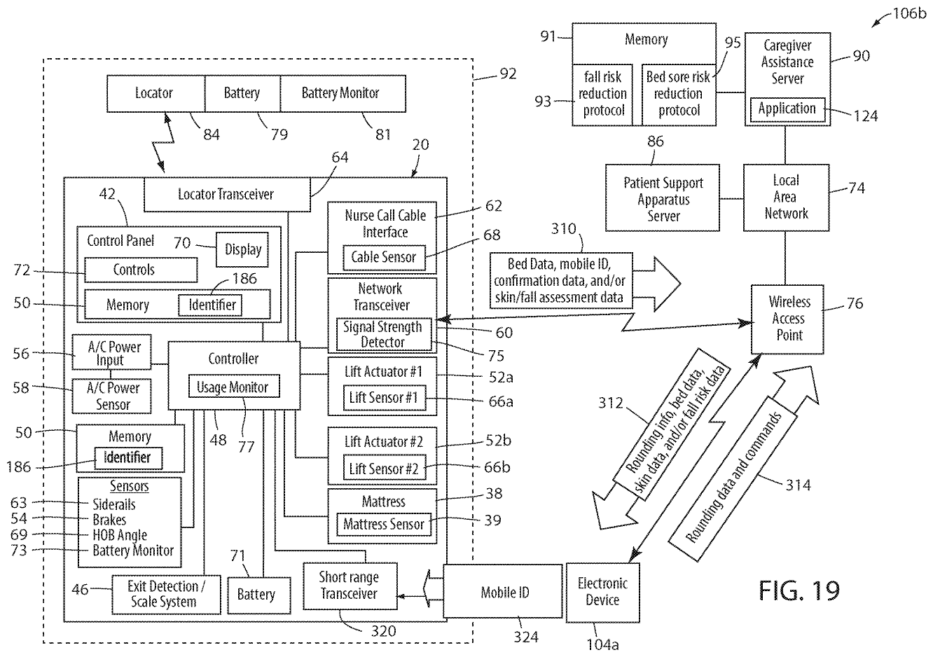

FIG. 19 illustrates a caregiver assistance system 106b according to another embodiment of the present disclosure. Caregiver assistance system 106b differs from caregiver assistance systems 106 and 106a of FIGS. 2 and 18, respectively, in that mobile electronic device 104a sends an electronic device message 324 to patient support apparatus 20a which then forwards it to caregiver assistance application 124, which uses it to establish the collocation of mobile electronic device 104a with that particular patient support apparatus 20. This collocation information is used by caregiver assistance application for multiple algorithms and multiple purposes. For example, with respect to rounding algorithm 140, the collocation information is used by patient support apparatus 20 to verify that the caregiver was present at the patient's bedside during the caregiver's performance of his or her rounding duties. With respect to risk reduction algorithms 141 and/or 143, the collocation information may be used to correlate the risk assessment to a specific patient or patient support apparatus 20, or it may be used to send a command to the patient support apparatus 20 directly in order to change a state of any of the bed sore risk components (e.g. mattress 38) or fall-risk components of patient support apparatus 20.

As shown more clearly in FIG. 19, mobile electronic device 104a is adapted in caregiver assistance system 106b to send out a short range message 324 to a nearby short range transceiver 320 of patient support apparatus 20a. The short range message 324 is sent as a result of any one or more of the following: in response to a user manipulating an input on mobile electronic device 104a, an expiration of a periodic time interval, an interrogation signal sent from short range transceiver 320 of patient support apparatus 20a, a signal from RTLS server 100 to mobile electronic device 104a indicating that it is currently in a room with one or more patient support apparatuses 20a, a combination of one or more of these triggering conditions, and/or in response to still other triggering conditions.

The content of electronic device message 324 includes a unique identifier that uniquely identifies the mobile electronic device 104a. This may be a serial number of the device 104a, a MAC address, or some other identifier that distinguishes that particular mobile electronic device 104a from other mobile or stationary electronic devices 104a, 104b that are part of system 106b, and/or other electronic devices that are not part of system 106b but which may utilize the same protocol and/or communication channel as transceiver 320.

As with patient support apparatus message 322 (FIG. 18), electronic device message 324 may be sent via infrared, near field communication, low power RF (e.g. Bluetooth), ultrasonic, or some other protocol that limits the range of message 324 such that it is not detected by patient support apparatuses 20a that are positioned outside of the room in which the caregiver is currently located.

Upon receipt, or shortly thereafter, of receiving the electronic device message 324, controller 48 of patient support apparatus 20a forwards a message to caregiver assistance application 124 informing application 124 of the receipt of the message 324, including the mobile ID contained within the message 324. Caregiver assistance application 124 uses the receipt of this information to conclude that, at that moment, the mobile electronic device is collocated in the same room as that particular patient support apparatus 20. When this collocation information is used with rounding algorithm 140, caregiver assistance application 124 waits for receipt of this message from patient support apparatus 20a after the caregiver has indicated he or she has completed a rounding task and, if it does not receive it, it concludes that there has been no verification of the caregiver's presence beside the patient when performing his or her rounding task. If the caregiver assistance application 124 receives the message, then it concludes that there has been verification of the task being completed.

In the caregiver assistance system 106b of FIG. 19, mobile electronic device 104a does not need to include any collocation data in the inbound messages 314 it sends to caregiver assistance server 90 because such collocation data is contained within the patient support apparatus messages 310 sent by network transceiver 60. In some embodiments, the collocation data contained within message 310 includes only the mobile electronic device ID, while in other embodiments, the collocation data includes additional information, such as, but not limited to, the time at which the electronic device message 324 was received. Of course, all of the messages 310 sent from patient support apparatus 20a (and patient support apparatuses 20) via network transceiver 60 to caregiver assistance server 90 include the patient support apparatus ID.

In the caregiver assistance system 106b of FIG. 19, the messages 314 sent by mobile electronic device 104a to caregiver assistance server 90 may omit patient support apparatus data that is used to determine whether the patient support apparatus 20a is in a compliant state or not. This information may be omitted because patient support apparatus 20a sends its status data directly via messages 310, and this status data is used by caregiver assistance application 124 to determine at step 254 whether the patient support apparatus 20 a is in a compliant state or not.

Caregiver assistance system 106b of FIG. 19 may be modified to replace the short range communication between mobile electronic device 104a and transceiver 320 of patient support apparatus 20a. In such modified embodiments, rather than having a wireless signal transmitted to patient support apparatus 20a to verify the colocation of the caregiver (or his or her mobile electronic device 104a) adjacent the patient support apparatus 20a, the patient support apparatus 20a is modified to accept a physical input from the caregiver, such as a button, switch, or the like, that the caregiver presses during the rounding task. The physical input may be included as an icon on a touchscreen of patient support apparatus 20a, or it may be a dedicated control, or it may some combination of the two. As an alternative to a physical input, a wireless signal may be utilized for collocation purposes that does not involve mobile electronic device 104a. For example, the input may involve a caregiver swiping a card with a magnetic strip along a card reader built into patient support apparatus 20a, or it may involve positioning a near field communication card adjacent a near field communication transceiver built into patient support apparatus 20a. Still other variations are possible.

Regardless of how the input to patient support apparatus 20 is implemented, when the caregiver physically or wirelessly activates the input on patient support apparatus 20a, controller 48 sends a message 310 to caregiver assistance application 124 that includes collocation data indicating that the caregiver was present adjacent patient support apparatus 20a. The message 310 may include a time at which the input was activated by the caregiver. In this modified embodiment of system 106b, short range transceiver 320 of patient support apparatus 20a may be omitted and/or modified, and mobile electronic device 104a need not include a transceiver that is compatible with transceiver 320.

It will be noted that, as shown in FIG. 19, caregiver assistance system 106b does not show electronic device 104 forwarding any fall risk assessment or bed risk assessment data to caregiver assistance server 90 via wireless access point 76. Although caregiver assistance system 106b can be configured to forward such data in the manner previously described, caregiver assistance system 106b can alternatively be configured such that this data, and/or any other data gathered by the caregiver, are input directly into patient support apparatus 20 via one or more of its control panels 42. Thus, instead of displaying screens on the display of an electronic device 104, caregiver assistance application 124 can be configured to work with a patient support apparatus 20 that displays screens on its own display that are the same as, or similar to, the electronic device 104 screens shown herein in the accompanying drawings. The data input via these screens is then sent by patient support apparatus 20 to caregiver assistance application 124 and used in the manner specified by the various algorithms. In this particular embodiment, the patient support apparatus 20 may be used to perform either or both of the risk assessments, and the electronic device 104 may be used to receive and display alerts if the fall risk reduction protocol and/or bed sore risk reduction protocol is not being followed. In still another alternative embodiment, one or more screens (e.g. the risk assessment screens, the servicing screens, the rounding screens, etc.) may be displayed on either or both of display 70 of patient support apparatus 20 and/or the display of the electronic device 104.

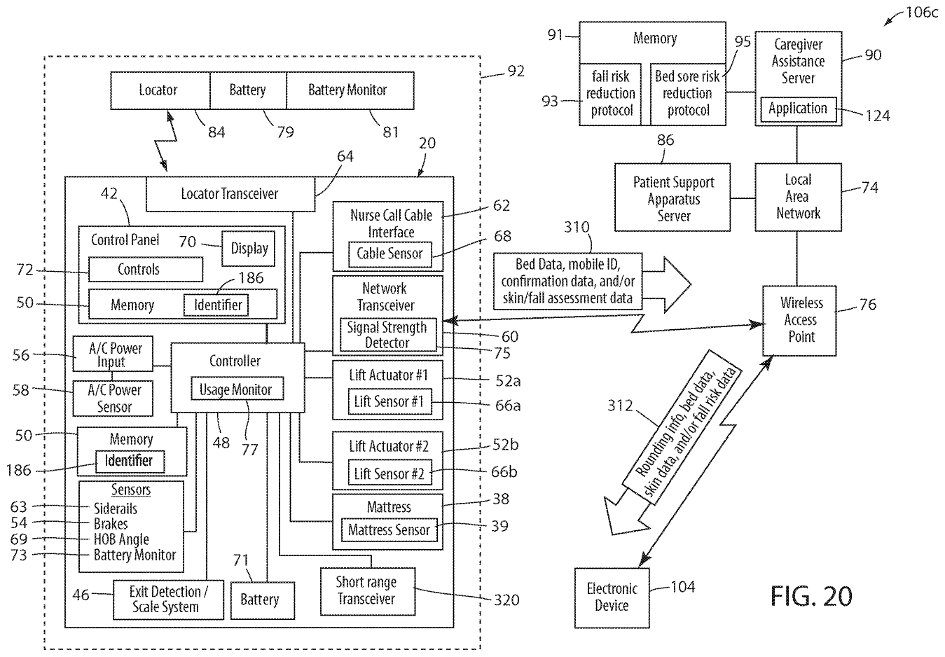

FIG. 20 illustrates another caregiver assistance system 106c according to yet another embodiment of the present disclosure. Caregiver assistance system 106c differs from caregiver assistance systems 106, 106a, and 106b of FIGS. 2, 18, and 19, respectively, in that mobile electronic device 104a does not send any rounding data, fall risk assessment data, bed sore risk assessment data, commands, or other data back to caregiver assistance application 124. Instead, such data is communicated to caregiver assistance application 124 via patient support apparatus 20a's communication channel with server 90. Caregiver assistance system 106c also differs from the other caregiver assistance systems 106, 106a, and 106b in that it can utilize either patient support apparatus 20 or patient support apparatus 20a. That is, the patient support apparatuses usable with caregiver assistance system 106c can include short range transceiver 320, or they may omit short range transceiver 320. Indeed, in some embodiments, caregiver assistance system 106c may be implemented in a healthcare facility wherein some of the patient support apparatuses includes short range transceiver 320 and others do not.

In the embodiment of FIG. 20, system 106c uses mobile electronic devices 104a (and/or stationary electronic devices 104b (not shown)) primarily to display information regarding the patient support apparatuses 20 and/or 20a, as well as, in some embodiments, to display rounding information, fall risk protocol compliance information, skin care protocol compliance information, and/or other information. The caregiver, however, does not utilize mobile electronic device 104a (or device 104b) to input rounding information, verification data, fall risk assessment data (e.g. answers to fall risk questions), bed sore risk assessment data (e.g. bed sore risk scores) and/or other data. Instead, any or all of this data is entered via a user interface of patient support apparatus 20 or 20a.

In the embodiment of FIG. 20, mobile electronic device 104a does not need to receive any compliance data from the patient support apparatus 20 or 20a because this information is sent from the patient support apparatus 20 to caregiver assistance application 124 (via messages 310). Indeed, in some embodiments of system 106c, mobile electronic devices 104a may be dispensed with entirely, or used only to receive alerts and/or status updates. Alternatively, mobile electronic devices 104a may be used to display a limited set of information (e.g. the rounding status and/or patient support apparatus status), but not accept any inputs regarding certain items of information (e.g. patient rounding, fall risk assessments, and/or commands for commanding a patient support apparatus.

In the embodiment of FIG. 20, patient support apparatus 20 or 20a may be configured to require a user to enter a username and/or a password before allowing the caregiver to input patient care data (e.g. rounding information, risk assessment data, documentation, etc.) into patient support apparatus 20 or 20a. Such access may be carried out in the same or similar manner to what is illustrated in FIGS. 6 and 7. Alternatively, in some embodiments, patient support apparatus 20 or 20a may be configured to allow the caregiver to enter such data without first establishing his or her credentials.

In the caregiver assistance system 106c of FIG. 20, both mobile electronic device 104a and patient support apparatus 20 (or 20a) may be configured to skip sending any collocation data to caregiver assistance server 90. This is because the patient care data comes to caregiver assistance server 90 via messages 310 from patient support apparatus 20 or 20a, and the content of those messages is the result of a user manually entering information into the patient support apparatus 20, 20a, which can only be done while the caregiver is collocated with the patient support apparatus 20, 20a. In other words, because such messages 310 are specifically received from patient support apparatus 20 or 20a, and are only sent in response to the caregiver manipulating one or more controls on the patient support apparatus 20 or 20a, the very sending of such messages 310 is verification that the caregiver is collocated adjacent the patient support apparatus 20 or 20a. Stated in yet another manner, because messages 310 originate from patient support apparatuses 20 or 20a in response to caregiver actions, such messages inherently provide their own verification of the caregiver's location.

In such embodiments, patient support apparatus 20 or 20a may be configured to send a message 310 whenever a button or control is activated on one of the caregiver control panels 42a or 42c. For example, if the scale controls are used to weigh the patient, or a therapy control is used to implement a mattress therapy, or the exit detection system is armed, controller 48 of patient support apparatus 20 or 20a sends a message 310 to caregiver assistance server indicating that a caregiver has activated a control on patient support apparatus 20 or 20a. The message 310 is sent because system 106c assumes that such button or control activations are the result of a caregiver's actions, not the patient's actions. As a result, the message 310 includes data indicating that a caregiver is present in the room. The message 310 may include data identifying the specific control that has been activated and/or a time at which the control was activated. Alternatively, message 310 may simply indicate that a caregiver control was activated without specifying which one and/or without specifying a time.

In some embodiments of system 106c, the caregiver carries a card (an RF ID card, a card with a magnetic strip, a near field communication card, or another type of card) that is detected by a corresponding sensor on the patient support apparatus 20 or 20a when the caregiver is within relatively close proximity to the patient support apparatus 20 or 20a (e.g. within the same room, or closer). In response to detecting the card, patient support apparatus 20 or 20a sends a message 310 to caregiver assistance application 124 indicating the presence of the caregiver, and caregiver assistance application 124 treats that message 310 as collocation data. As noted, the collocation data may be used for a number of different purposes including, but not limited to, proof that the caregiver has completed a round with the patient. The message 310 may also include patient support apparatus data that caregiver assistance application 124 uses to determine if the patient support apparatus 20 or 20a is in a compliant or non-compliant state. This data (the compliancy data and rounding completion data) is then sent to EMR server 98 as part of algorithm 140.

FIG. 21 illustrates another caregiver assistance system 106d according to yet another embodiment of the present disclosure. Caregiver assistance system 106d differs from caregiver assistance systems 106, 106a, 106b, and 106c of FIGS. 2, 18, 19, and 20, respectively, in that it includes a remote equipment management interface 922 that is adapted to enable caregiver assistance application 124 to work in conjunction with a standalone remote equipment management system 918. The remote equipment management system 918 may be a conventional, standalone equipment management system that repetitively receives updates from equipment within the healthcare facility (e.g. patient support apparatuses 20) regarding their usage, maintenance, servicing, battery state, etc. In at least one embodiment, the remote equipment management system 918 is implemented in any of the forms disclosed in commonly assigned PCT patent publication WO 2018-013666 filed Jul. 12, 2017, by inventors David Becker et al. (Int'l app. #: PCT/US 2017/041681) and entitled EQUIPMENT MANAGEMENT SYSTEM, the complete disclosure of which is incorporated herein by reference in its entirety. Caregiver assistance application 124 may be configured to interface with other equipment management systems as well.

As shown in FIG. 21, remote equipment management system 918 includes one or more remote equipment management servers 920 (positioned outside of a healthcare facility 940) that communicate with one or more pieces of medical equipment, such as patient support apparatuses 20. Remote equipment management server 920 communicates with the equipment using various structures of the healthcare facility's local area network 74, such as, but not limited to, patient support apparatus server 86, wireless access points 76, and internet gateway 108 that couples local area network 74 to the Internet 110. Remote equipment server 920 is located at an Internet accessible location that is therefore able to communicate with LAN 74 via gateway 108.

Each patient support apparatus 20 that is part of remote equipment management system 918 is configured to send its data to patient support apparatus server 86. Patient support apparatus server 86 is configured to allow one or more services to subscribe to the patient support apparatus data that it receives. In the embodiment shown in FIG. 21, both caregiver assistance application 124 and remote equipment management system 918 include a subscription to this patient support apparatus data. Thus, as a result of a caregiver assistance application subscription 924, patient support apparatus server 86 forwards patient support apparatus data to server 90 and caregiver assistance application 124. This data includes the data described above, such as, but not limited to, the data displayed in the top portion 202 of various of the caregiver assistance screens. As a result of a remote equipment management system subscription 926, patient support apparatus server 86 also sends the patient support apparatus data it receives from patient support apparatuses 20 to remote equipment management server 920.

It will be understood that the subscriptions 924 and 926 may be for the same sets of patient support apparatus data, or they may include subscriptions to different data sets. Still further, it will be understood that, in some embodiments, patient support apparatuses 20 may be configured to send their patient support apparatus data to remote equipment management system server 920 directly (i.e. without passing through server 86).

In the embodiment shown in FIG. 21, the patient support apparatus data travels from a patient support apparatus 20 to patient support apparatus server 86 along a first path 928. After arriving at patient support apparatus server 86, the data is forwarded to server 90 and caregiver assistance application 124 via a second path 930. Further, the patient support apparatus data received at patient support apparatus server 86 (or a subset of it) is forwarded by server 86 to remote equipment management server 920 via a third path 932. The data that is stored at remote equipment management server 920, or a subset of it, is accessible to users of the remote equipment management system 918 via one or more electronic devices 104c. Electronic devices 104c include smart phones, tablets, laptops, desktops, and browser enabled displays. Electronic devices 104c may be the same as any of devices 104a and/or 104b, or they may be different. Whether the same or different, a direct user of remote equipment management system 918 uses the electronic device 104c to access remote equipment management server 920 by navigating to a specific remote equipment management URL 960. In contrast, a user of caregiver assistance system 106d accesses caregiver assistance application 124 by navigating to a separate caregiver assistance application URL 962. For each URL, the user must present separate login credentials.

Thus, for a user to access the equipment management data stored at server 920 (the contents of which are described in greater detail in the aforementioned PCT patent publication WO 2018-013666), the user enters valid login information (e.g. username and password) into the electronic device 104c and that information is communicated directly (via access point 76, gateway 108, and Internet 110) to server(s) 920. If the login information is valid, the user is able to view the equipment management data stored at server 920 on his or her electronic device 104c. This data is communicated to the electronic device 104c via a fourth path. As shown in FIG. 21, fourth path 934 bypasses caregiver assistance application 124 and server 90.

Because remote equipment management system 918 is a separate system that utilizes different users with different login information, and because it stores different sets of data than caregiver assistance application 124, a user who directly accesses server 920 via electronic device 104c is not able to access any of the data of caregiver assistance application 124. Similarly, a user of caregiver assistance application 124 is not able to send his or her caregiver assistance system login information to server 920 in order to gain access to the data stored therein. However, as will now be discussed, caregiver assistance server 90 of system 106d is constructed so that users of caregiver assistance application 124 are able to gain access to the data of remote equipment management system 918.

In order for a user of caregiver assistance application 124 to access the data of remote equipment management system 918, he or she must be validly logged into caregiver assistance application 124. Once logged in there, he or she may send a request for patient support apparatus data to caregiver assistance application 124. Caregiver assistance application 124 receives this request and uses remote equipment management communication interface 922 to communicate the request to remote equipment management server 920. Interface 922 is configured with a list of authorized users who are permitted to access remote equipment management system 918, as well as the communication protocols used to communicate with remote equipment management server 920. Further, interface 922 includes, or has access to, the IP address(es) and/or URL(s) of remote equipment management server(s) 920. Caregiver assistance application 124 is therefore able to act as an authorized user of remote equipment management system 918 for all of, or a set of, the users of caregiver assistance application 124. This allows those user to access data from remote equipment management server 920. Further, in some embodiments, caregiver assistance application 124 is configured to allow an authorized user of caregiver assistance application 124 to upload data, such as, but not limited to, servicing data to remote equipment management server(s) 920.

The data communicated between caregiver assistance server 90 and remote equipment management server 920 follows a fifth path 936. As a result, if a user of caregiver assistance application 124 is logged into caregiver assistance application 124 on a mobile electronic device 104a (see FIG. 21), he or she is able to access equipment data stored at server 920 by sending a request to server 90. The request is forwarded via fifth data path 936 to server 920, and server 920 responds to server 90 via fifth path 936. This response is then forwarded to mobile electronic device 104a via local area network 74 and an appropriate wireless access point 76. In contrast, if a user is logged into remote equipment management system 918 directly on his or her electronic device 104c, they are able to access the data of remote equipment management server 920 by bypassing server 90 and caregiver assistance application 124. That is, the communication between electronic device 104c and server 920 takes place via path 934, and path 934 does not utilize caregiver assistance application 124 or server 90.

Interface 922 of caregiver assistance application 124 therefore allows users of caregiver assistance application 124 to utilize one or more electronic devices 104a or 104b to access data stored at remote equipment management system 918 without having to separately log into system 918, or to manually input any other data necessary for viewing this data. As a result, a single user interface of electronic devices 104a, 104b is able to provide the user with the functionality of both caregiver assistance application 124 and remote equipment management system 918. The user therefore does not need to download separate applications to utilize both systems, log into separate systems, and/or direct their web browser to separate URL's in order to enjoy the advantages of both systems.

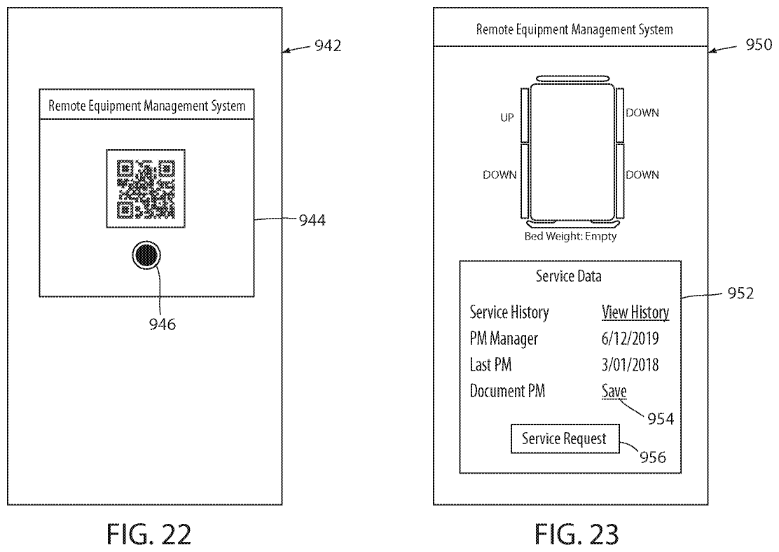

FIG. 22 depicts one example of a manner in which a user of caregiver assistance application 124 can utilize his or her electronic device 104a to gain access to the data stored at remote equipment management server 920. More particularly, FIG. 22 depicts a remote equipment management system access screen 942 that includes an image window 944 and a capture icon 946. Image window 944 and capture icon 946 may operate to allow the caregiver to capture an image of a QR code, or other unique visual identifier on a particular patient support apparatus 20. After capturing the image, the image is either locally processed on device 104a to determine the identity of the corresponding patient support apparatus 20, or it is sent to caregiver assistance application 124 to determine the identity of the corresponding patient support apparatus 20. After the identity of the patient support apparatus 20 is determined, caregiver assistance application 124 sends a request to remote equipment management server 920 for data corresponding to that particular patient support apparatus 20. Server 920 sends the requested data back to caregiver assistance application 124 via path 936 and caregiver assistance application 124 then displays this data (or a portion of it) on the screen of the mobile electronic device 104a.

As an alternative to the access method shown in FIG. 22, the user may gain access to the data contained within remote equipment management server 920 for a particular patient support apparatus 20, 20a by simply pressing on, or otherwise activating a control (not shown), that is displayed on his or her mobile electronic device 104a. Caregiver assistance application 124 then automatically selects a particular patient support apparatus 20, 20a and retrieves the service data from server 920 for the selected patient support apparatus 20, 20a. Caregiver assistance application 124 makes this automatic selection based on the current collocation data for the caregiver's mobile electronic device 104a. That is, it determines which patient support apparatus 20, 20a the caregiver's mobile electronic device 104a is currently collocated with, and selects that particular patient support apparatus 20, 20a. Alternatively, or additionally, caregiver assistance application 124 may automatically select whichever patient support apparatus 20, 20a has its status data currently displayed on the display of the caregiver's mobile electronic device 104a (e.g. on the room overview screen 162, see FIG. 8).

FIG. 23 illustrates one example of a remote equipment management data display screen 950. Remote equipment management data display screen 950 displays patient support apparatus data that is obtained from remote equipment management server(s) 920. More particularly, screen 950 includes a service data window 952 that displays various data regarding the servicing of the patient support apparatus 20. This service data includes, but is not limited to, data indicating the service history of that patient support apparatus, data indicating the last time preventative maintenance was performed on the patient support apparatus 20, and/or data indicating who is responsible for performing one or more types of service on the patient support apparatus 20.

In addition, screen 950 may also include a “document” icon 954 and a “service request” icon 956. The “document” icon 954 is touched when the user has performed a particular service on the patient support apparatus 20. Pressing this icon causes caregiver assistance application 124 to send information to remote equipment management server 920 indicating that that particular patient support apparatus 20 was serviced. This information includes, but is not limited to, the type of service performed, the person who performed the server, and the date (and time) of the service.

The “service request” icon 956 allows caregivers to request service and/or report errors or damage to hospital equipment, such as, but not limited to, patient support apparatuses. Such reports and/or requests are automatically forwarded to the remote equipment management server 920, which is in communication with one or more appropriate service personnel. In some embodiments, “service request” icon 956 also allows the caregiver to send digital images of, and/or textual information regarding, the equipment that is in need of service. This image data and/or textual data is forwarded to server 920 for communication to appropriate service personnel.

More specifically, if the user selects the “service request” icon 956, caregiver assistance application 124 sends a message to remote equipment management server 920 indicating that service has been requested for that particular patient support apparatus 20. Server 920 forwards this message to appropriate personnel, as described in more detail in the aforementioned PCT patent publication WO 2018-013666).

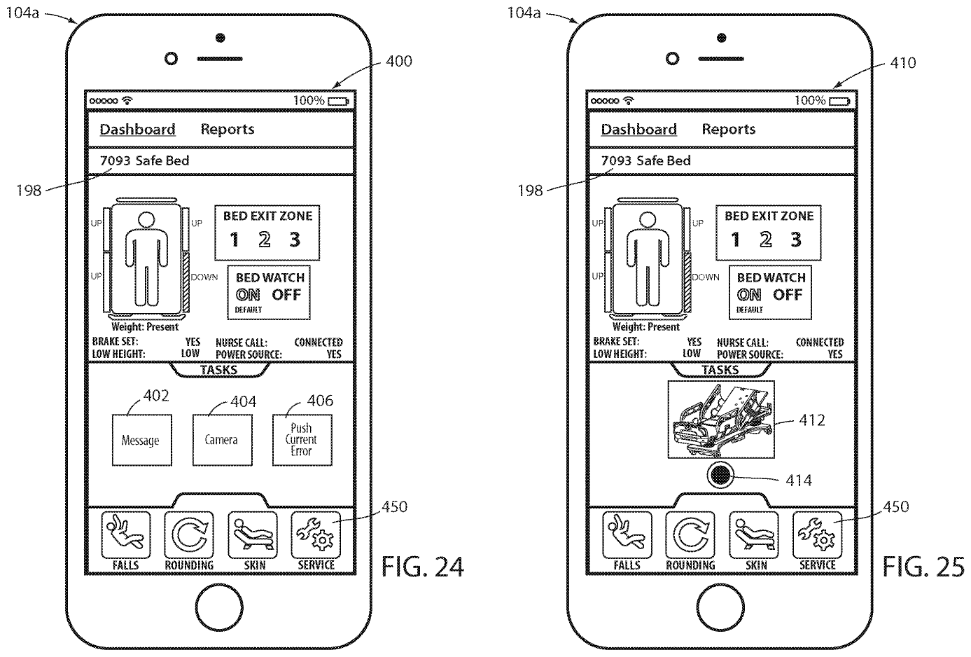

In some embodiments, the activation of the “service request” icon 956 causes caregiver assistance application 124 to display a first service request screen 400, such as that shown in FIG. 24. First service request screen 400 includes a message icon 402, a camera icon 404, and a push error icon 406. The message icon 402 is to be selected by the user if the user wishes to enter textual information into a service request. The camera icon 404 is to be selected by the user if the user wishes to take one or more digital images of all, or a portion, of the patient support apparatus to which the service request pertains (e.g. an image of a broken component of the patient support apparatus). The push error icon 406 is to be selected by the user if the patient support apparatus is displaying an error message on one of its displays and the caregiver wishes to command the patient support apparatus to forward that error message to the remote equipment management server 920.

If the user selects camera icon 404 of first service request screen 400, caregiver assistance application 124 may be configured to display a second service request screen 410, such as is shown in FIG. 25. Second service request screen 410 includes an image window 412 and an image capture icon 414. Image window 412 displays the current image being sensed by the built-in camera of mobile electronic device 104a. After the user has correctly aligned and focused the built-in camera on the patient support apparatus needing service (or a component thereof), he or she presses the image capture icon 414 and the camera of mobile electronic device 104a captures the current image shown in image window 412.

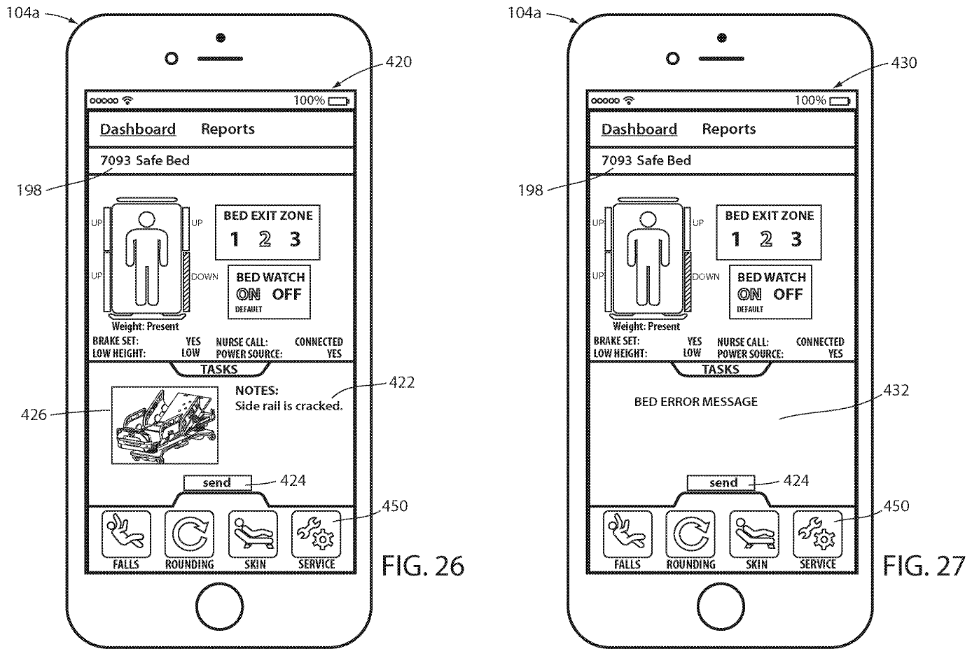

After the user has captured one or more images of the patient support apparatus (or a portion thereof), caregiver assistance application is configured, in at least one embodiment, to display a third service request screen 420, such as is shown in FIG. 26. Third service request screen 420 includes a text addition area 422, a send icon 424, and a captured image window 426. Captured image window 426 displays the image(s) that were captured by the user using image capture screen 410 (FIG. 25). Text addition area 422 is an area where the user can type any desired explanatory text that he or she wishes to accompany the captured image. In the example illustrated, the user has added the text message “side rail is cracked.” The send icon 424 is adapted to be pressed by the user when he or she is ready to send the service request to the remote equipment management server 920. That is, in response to the user touching send icon 424, mobile electronic device 104a sends a message containing both the captured image and any accompanying text to the remote equipment management server 920.

The message may also include, or be accompanied by, collocation data sent from mobile electronic device 104a so that caregiver assistance application 124 can automatically associate the image and/or text with a particular patient support apparatus 20, 20a. Alternatively, such collocation data may be sent to caregiver assistance application 124 via the patient support apparatus 20, 20a. As described previously, the collocation data indicates which specific patient support apparatus 20, 20a the mobile electronic device 104a was collocated with at, or near, the time the image was captured, and/or at, or near, the time the send icon 424 was activated by the user. As another alternative, such collocation data may be omitted, and caregiver assistance application 124 may automatically associated the message with the particular patient support apparatus in the room that was identified in the room identifier location 198 on screen 440 (and/or screens 400, 410, 420, and/or 430) at the time the message was sent.

The message may also include an identification of the person making the request, which caregiver assistance application 124 may automatically append to the message based on its knowledge of which mobile electronic devices 104a are associated with which persons. Still further, the message may include a time stamp indicating when the service request was made, as well as a current location of the patient support apparatus 20, 20a to which the service request pertains. Still other information may be included in the message.

If the user selects the push error icon 406 of first service request screen 400, caregiver assistance application 124 may be configured to display a fourth service request screen 430, such as is shown in FIG. 27. Fourth service request screen 430 includes an error push window 432 and a send icon 424. The error push window 432 may display whatever error notification the patient support apparatus 20 is currently displaying on one or more of its displays. This information is communicated to mobile electronic device 104a either directly (e.g. via short range transmitter 320) or by patient support apparatus 20, 20a sending the data to caregiver assistance application 124, which then forwards it to mobile electronic device 104a. In some embodiments, error message window 432 may be adapted to allow the user to add additional explanatory text to the error message, if desired. If the user wishes to send this error notification (and any additional explanatory text) to remote equipment management server 920, he or she presses send icon 424. Mobile electronic device 104a then sends this error message to caregiver assistance application 124 which, in turn, forwards it to remote equipment management server 920 (or mobile electronic device 104a may send it directly to remote equipment management server 920). The sending of this error message may be accompanied by any of the aforementioned data (e.g. requestor ID, location, time and date, etc.) and/or any of the aforementioned collocation data that allows caregiver assistance application 124 to automatically associate the service request to a specific patient support apparatus 20. Or, as noted above, the specific patient support apparatus 20, 20a may be identified in the message based on the room identifier shown on screen 430 at the time the service request is sent, as was discussed above.

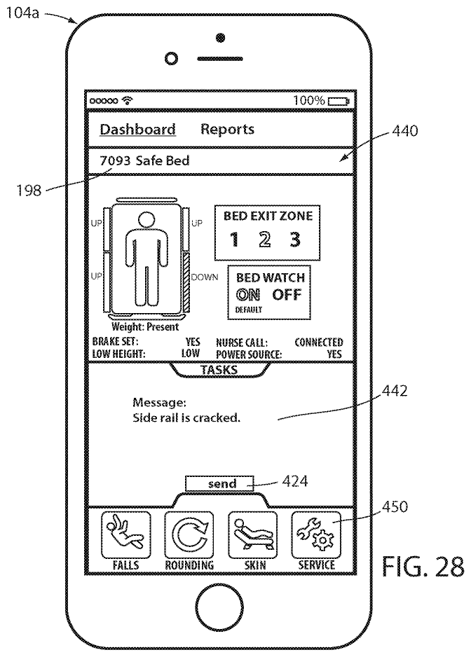

If the user selects the message icon 402 of first service request screen 400, caregiver assistance application 124 may be configured to display a fifth service request screen 440, such as is shown in FIG. 28. Fifth service request screen 440 includes a message window 442 and the send icon 424. The message window 442 allows the user enter whatever explanatory textual information he or she wishes to include with the service request. Such text, for example, may identify what component needs servicing, what the problem is with one or more components, and/or any other information that may be helpful for a service person to know about. When the user is ready to send the service request with whichever text he or she has chosen, he or she presses the send icon 424. The service request is then sent in the manner described above and may, or may not, be accompanied by any of the aforementioned data, as mentioned above.

It will be noted that screens 400, 410, 420, 430, and 440 all include a service task icon 450 contained within their task menu 174. In some embodiments, this service task icon 450 is added to the task icons 178, 180, 182, and 184 (see, e.g. FIG. 8) so that task menu 174 includes five task icons. In other embodiments, service task icon 450 may replace one of these other task icons 178, 180, 182, and/or 184. When the user selects service task icon 450, caregiver assistance application 124 is configured to display one or more screens that relate to service requests and/or the servicing of one or more patient support apparatuses 20, 20a, or related equipment. Thus, in some embodiments, the screens shown in FIGS. 22-29 are accessed by the user after he or she presses on service task icon 450.

In addition to, or in lieu of, interfacing caregiver assistance system 106d with a remote equipment management system 918, any of the caregiver assistance systems 106, 106a, 106b, 106c, and/or 106d described herein may be further modified to interface with a patient support apparatus configuration system. In such systems, the mobile electronic device 104a may be used as a configuration tool that enables the user to change configuration settings onboard one or more of the patient support apparatuses 20. In such embodiments, caregiver assistance application 124 allows the user of a mobile electronic device 104a to use the mobile electronic device 104a as a configuration tool in any of the same manners as the configuration tools are used in commonly assigned U.S. patent application Ser. No. 16/057,928 filed Aug. 8, 2018, by inventors Marco Constant et al. and entitled FIELD CONFIGURABLE PATIENT SUPPORT APPARATUSES, the complete disclosure of which is incorporated herein by reference.

For example, in this modified embodiment, caregiver assistance application 124 may be configured to interface with the information system 174 disclosed in the aforementioned '928 application. Interfacing with system 174 enables mobile electronic device 104a to receive configuration settings for a particular patient support apparatus 20 and to transfer those to the particular patient support apparatus 20. Further, this interfacing with a configuration system allows a user of caregiver assistance application 124 to use his or her mobile electronic device 104a to configure the patient support apparatus 20 to communicate with a specific type of nurse call system; and/or input desired presets into the patient support apparatus 20 (e.g. default transfer height, default bed monitoring system settings, default bed locking/unlocking settings, etc.). Indeed, in some of these embodiments, caregiver assistance application 124 is configured to allow a user to send messages to a patient support apparatus 20 that enables the patient support apparatus 20 to carry out any of the configuration changes that are disclosed in commonly assigned U.S. patent application Ser. No. 16/272,332 filed Feb. 11, 2019, by inventors Krishna Bhimavarapu et al. and entitled PATIENT SUPPORT APPARATUSES WITH RECONFIGURABLE COMMUNICATION, and/or any of the remote control commands or configuration changes disclosed in commonly assigned U.S. patent application Ser. No. 63/026,937 filed May 19, 2020, by inventors Alexander Bodurka et al. and entitled PATIENT SUPPORT APPARATUSES WITH HEADWALL COMMUNICATION, the complete disclosures of both of which are incorporated herein by reference.

It will be understood by those skilled in the art that any of the components, functions, and/or features of the different embodiments of caregiver assistance systems 106, 106a, 106b, 106c and 106d may be combined together, substituted, and/or mixed in any manner. As but one non-limited example, system 106 may be modified to omit the display of any rounding questions of algorithm 140, similar to modified system 106c, and the patient support apparatuses 20 of system 106 may be modified to display a code that identifies the bed and the current time. In this modified system, the caregiver is assumed to ask the desired rounding questions and take care of the desired rounding tasks, and the modified system merely verifies the caregiver's presence in the patient's rooms. This presence is verified by the modified patient support apparatus displaying the code and the caregiver capturing an image of this code using his or her mobile electronic device 104a that sends the captured image to caregiver assistance server 90. In some embodiments, the code includes both the bed ID and time, while in other embodiments the code includes only the bed ID. In still other embodiments, the bed ID and/or time are not coded at all, but merely displayed so that an image of them can be captured by the caregiver's mobile electronic device 104a. In a variation on this embodiment, the patient support apparatus 20 may be configured to not display the ID and/or time (or the code) or the patient support apparatus ID if the patient support apparatus is not currently in a compliant state, or it may simultaneously display the fact that it is not in a compliant state along with the ID and/or time (or a code with such information).

It will also be understood that, in any of the embodiments discussed above that utilize one or more near field transceivers incorporated into any of the patient support apparatuses 20 or 20a, such patient support apparatuses 20 or 20a may be constructed to include such near field transceivers and/or utilize the near field transceivers in any of the manners disclosed in commonly assigned U.S. Pat. No. 9,966,997 issued May 8, 2018, to inventors Michael Hayes et al. and entitled COMMUNICATION SYSTEMS FOR PATIENT SUPPORT APPARATUSES, the complete disclosure of which is incorporated herein by reference.

Still further, it will be understood that any of the embodiments of the caregiver assistance system may omit one or more of the algorithms shown in FIG. 3, and/or these algorithms may be supplemented with additional algorithms. Thus, for example, in some modified embodiments, caregiver assistance system 106 (or 106a, 106b, 106c, or 106d) is only configured to implement fall risk reduction algorithm 143 without implementing any of the rounding algorithm 140, skin care algorithm 141, documentation algorithm 800, wireless connection algorithm 990, manual task list algorithm 151, reminder algorithm 145, and/or sharing algorithm 175. In still other embodiments, still other combinations of two or more of the algorithms shown in FIG. 3 may be implemented by the system. Further, it will be understood that additional modifications may be made to individual algorithms beyond those already discussed above.

Still further, in any of the embodiments discussed above, caregiver assistance application 124 may be modified to prevent rounding data, or other patient data, to be entered until an overdue task is completed, such as, but not limited to, one or both of the fall risk and bed sore risk assessments. Additionally, or alternatively, in any of the embodiments discussed above, any of the data that is shown on the screens of electronic devices 104 may alternatively or additionally be shown on the screen of the display 70 of patient support apparatus 20. Thus, for example, if a patient is determined to be a fall risk, that fall risk category may be added to the display of the corresponding patient support apparatus 20 so that the caregiver is reminded of the patient being a fall risk whenever he or she uses the control panel 42 of the patient support apparatus. Still further, indicators may be added to the screens of mobile electronic devices 104a and/or patient support apparatuses 20a whenever they are in sufficient proximity to communicate with each other using short range transceiver 320.

It will also be understood that any of the embodiments of caregiver assistance system 106, 106a-d may be further modified to display additional screens beyond those described above, and/or that the screens described herein may be modified and/or replaced with other screens.

Various additional alterations and changes beyond those already mentioned herein can be made to the above-described embodiments. This disclosure is presented for illustrative purposes and should not be interpreted as an exhaustive description of all embodiments or to limit the scope of the claims to the specific elements illustrated or described in connection with these embodiments. For example, and without limitation, any individual element(s) of the described embodiments may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Any reference to claim elements in the singular, for example, using the articles “a,” “an,” “the” or “said,” is not to be construed as limiting the element to the singular.

What is claimed is:

1. A patient support apparatus system comprising a plurality of patient support apparatuses, a server, and a mobile electronic device;
wherein each of the patient support apparatuses comprise:
a litter frame;
a support deck supported on the litter frame and configured to support a patient thereon;
a memory containing a unique identifier uniquely identifying the patient support apparatus;
an alert subsystem adapted to issue an aural alert in response to an alert condition being detected onboard the patient support apparatus;
an ultrasonic transmitter;
a first network transceiver adapted to communicate with the server;
a controller in communication with the memory and the first network transceiver, the controller adapted to transmit a first network message containing the unique identifier to the server using the first network transceiver and to transmit an ultrasonic message containing the unique identifier to the mobile electronic device using the ultrasonic transmitter;
wherein the mobile electronic device is adapted to receive the ultrasonic message and forward the unique identifier contained within the ultrasonic message to the server in a second network message using a second network transceiver integrated into the mobile electronic device;
wherein the server is adapted to select a particular patient support apparatus from amongst the plurality of patient support apparatuses having the same unique identifier as the unique identifier received in the second network message, the server further adapted to automatically send a volume control message to the particular patient support apparatus in response to receiving the second network message; and
wherein the controller of the particular patient support apparatus is adapted to automatically reduce a volume level of the aural alert in response to receiving the volume control message.

2. The patient support apparatus system of claim 1 wherein the server is adapted to send the volume control message to the particular patient support apparatus along a communication pathway that excludes the mobile electronic device.

3. The patient support apparatus system of claim 1 wherein the server is adapted to send the volume control message to the particular patient support apparatus along a communication pathway that includes the mobile electronic device.

4. The patient support apparatus system of claim 1 wherein the controller is adapted to transmit the ultrasonic message without requiring a user of the mobile electronic device to manually manipulate any controls on the particular patient support apparatus.

5. The patient support apparatus system of claim 1 wherein the mobile electronic device further includes a camera adapted to capture an image of the patient, the mobile electronic device is adapted to transmit the image to the server, and the server is adapted to automatically select a particular patient assigned to the particular patient support apparatus without requiring a user to input any information into the mobile electronic device identifying the particular patient.

6. The patient support apparatus system of claim 5 wherein the mobile electronic device is adapted to not transmit to the server any personal identification data that identifies the particular patient.

7. The patient support apparatus system of claim 6 wherein the mobile electronic device is further adapted to automatically delete the image after transmitting the message to the server.

8. The patient support apparatus system of claim 5 wherein the mobile electronic device is further adapted to use the image to measure a physical dimension of a portion of the particular patient's skin captured in the image.

9. The patient support apparatus system of claim 8 wherein the mobile electronic device is further adapted to measure the physical dimension by measuring a distance between the particular patient's skin and the mobile electronic device.

10. The patient support apparatus system of claim 1 wherein the mobile electronic device is further adapted to present a service request option on a display of the mobile electronic device, wherein the service request option is adapted to allow a user of the mobile electronic device to make a service request for the particular patient support apparatus to the server, and the server is further adapted to forward the service request to a service server, the service server adapted to forward the service request to at least one service person.

11. The patient support apparatus system of claim 10 wherein the server is further adapted to identify the particular patient support apparatus to which the service request pertains by utilizing service correlation data associating the service request with the particular patient support apparatus.

12. The patient support apparatus system of claim 10 wherein the service request option automatically corresponds to a first patient support apparatus while the mobile electronic device is positioned in a first room and automatically corresponds to a second and different patient support apparatus while the mobile electronic device is positioned in a second and different room.

13. The patient support apparatus system of claim 1 wherein the server is further adapted to instruct the mobile electronic device to display rounding data thereon, the rounding data indicating at least one of: an amount of time since a caregiver last completed a rounding task associated with a particular patient assigned to the particular patient support apparatus, or an amount of time until the caregiver is supposed to complete a future rounding task associated with the particular patient.

14. The patient support apparatus system of claim 13 wherein the server is further adapted to perform the following:
(i) receive from the mobile electronic device a digital image of a particular patient's skin captured by a camera onboard the mobile electronic device;
(ii) identify the particular patient support apparatus utilizing correlation data associating the digital image with the particular patient support apparatus; and
(iii) send a health records message to an electronic medical records server in communication with the server, the health records message including the digital image and a patient identifier uniquely identifying the particular patient.

15. The patient support apparatus system of claim 14 wherein the server is further adapted to receive the correlation data from the assigned patient support apparatus.

16. The patient support apparatus system of claim 14 wherein the server is further adapted to receive the correlation data from the mobile electronic device.

17. The patient support apparatus system of claim 1 wherein the particular patient support apparatus is further adapted to detect a presence of the mobile electronic device in a common room by receiving a wireless signal directly from the mobile electronic device.

18. The patient support apparatus system of claim 17 wherein the wireless signal contains the unique identifier of the particular patient support apparatus.

19. The patient support apparatus of claim 1 wherein the controller is adapted to repetitively transmit the ultrasonic message using the ultrasonic transmitter.

20. The patient support apparatus of claim 1 wherein the server is adapted to automatically send a second volume control message to the particular patient support apparatus in response to the mobile electronic device no longer receiving the ultrasonic message from the ultrasonic transmitter, and wherein the second volume control message increases the volume of the aural alert if the alert condition is still detected.
